(19) United States
(12) Patent Application Publication
(10) Pub. No.: US 2026/0358345 A1
(43) Pub. Date: Nov. 12, 2026

(54) METHOD

(71) Applicant: Oxford Nanopore Technologies PLC, Oxford (GB)

(72) Inventors: Clive Gavin Brown, Cambridge (GB); James Edward Graham, Oxford (GB); Richard Alexander Gutierrez, Oxford (GB); Jaskiran Singh Sabharwal, Oxford (GB); James White, Oxford (GB)

(73) Assignee: Oxford Nanopore Technologies PLC, Oxford (GB)

(21) Appl. No.: 19/125,616

(22) PCT Filed: Oct. 31, 2023
(86) PCT No.: PCT/GB2023/052838

§ 371 Date: Apr. 29, 2025

(30) Foreign Application Priority Data
Oct. 31, 2022 (GB) 2216162.4

(51) Int. Cl. G01N 33/68; G01N 33/487

(52) U.S. Cl. CPC G01N 33/6818; G01N 33/48721

(57) ABSTRACT

Provided herein are methods of characterising a target polypeptide as it moves with respect to a nanopore. Also provided are related kits, systems and apparatuses for carrying out such methods.

### FIELD

The present disclosure relates to methods of characterising a target polypeptide by forming a construct comprising a first strand comprising the target polypeptide and a second strand such as a polynucleotide strand, moving the construct with respect to a nanopore under conditions such that the first strand and the second strand of the construct move through the nanopore, and taking measurements characteristic of the polypeptide during such movement. The disclosure also relates to kits, systems and apparatuses for carrying out such methods.

### BACKGROUND

The characterisation of biological molecules is of increasing importance in biomedical and biotechnological applications. For example, sequencing of nucleic acids allows the study of genomes and the proteins they encode and, for example, allows correlation between nucleic acid mutations and observable phenomena such as disease indications. Nucleic acid sequencing can be used in evolutionary biology to study the relationship between organisms. Metagenomics involves identifying organisms present in samples, for example microbes in a microbiome, with nucleic acid sequencing allowing the identification of such organisms. Whilst techniques to characterise (e.g. sequence) polynucleotides have been extensively developed, techniques to characterise polypeptides are less advanced, despite being of very significant biotechnological importance. For example, knowledge of a protein sequence can allow structure-activity relationships to be established and has implications in rational drug development strategies for developing ligands for specific receptors. Identification of post-translational modifications is also key to understanding the functional properties of many proteins. For example, typically 30-50% of protein species are phosphorylated in eukaryotes. Some proteins may have multiple phosphorylation sites, serving to activate or inactivate a protein, promote its degradation, or modulate interactions with protein partners.

### SUMMARY

Known methods of characterising polypeptides include mass spectrometry and Edman degradation.

Protein mass spectrometry involves characterising whole proteins or fragments thereof in an ionised form. Known methods of protein mass spectrometry include electrospray ionisation (ESI) and matrix-assisted laser desorption/ionisation (MALDI). Mass spectrometry has some benefits, but results obtained can be affected by the presence of contaminants and it can be difficult to process fragile molecules without their fragmentation. Moreover, mass spectrometry is not a single molecule technique and provides only bulk information about the sample interrogated. Mass spectrometry is unsuitable for characterising differences within a population of polypeptide samples and is unwieldy when seeking to distinguish neighbouring residues.

Edman degradation is an alternative to mass spectrometry which allows the residue-by-residue sequencing of polypeptides. Edman degradation sequences polypeptides by sequentially cleaving the N-terminal amino acid and then characterising the individually cleaved residues using chromatography or electrophoresis. However, Edman sequencing is slow, involves the use of costly reagents, and like mass spectrometry is not a single molecule technique.

As such, there remains a pressing need for new techniques to characterise polypeptides, especially at the single molecule level. Single molecule techniques for characterising biomolecules such as polynucleotides have proven to be particularly attractive due to their high fidelity and avoidance of amplification bias.

One attractive method of single molecule characterization of biomolecules such as polypeptides is nanopore sensing. Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore. Nanopore sensing has the potential to allow rapid and cheap polypeptide characterisation.

Nanopore sensing and characterisation of polypeptides has been proposed in the art. For example, WO 2013/123379 discloses the use of an NTP-driven protein processing unfoldase enzyme to process a protein to be translocated through a nanopore. WO 2021/111125 discloses methods in which a target polypeptide is conjugated to a polynucleotide to form a single-stranded polypeptide-polynucleotide conjugate, with the conjugate being moved through a nanopore using a polynucleotide-handling protein. However, there remains a need for alternative and/or improved methods of characterising polypeptides.

The methods disclosed herein can also be applied to the characterisation of polynucleotides as described below.

The disclosure relates to methods of characterising a target polypeptide.

In one aspect, the methods involve a conjugate strand which comprises the target polypeptide. The target polypeptide is conjugated at each end of the polypeptide to one or more polynucleotide flanking strands. The conjugate is contacted with a polynucleotide carrier strand thereby forming a polynucleotide-polypeptide construct. The construct is contacted with a nanopore. The contacting takes place under conditions such that the polynucleotide-polypeptide conjugate strand and the polynucleotide carrier strand both co-translocate through the nanopore. One or more measurements characteristic of the polypeptide are taken as the conjugate moves with respect to the nanopore. In this manner, the target polypeptide which is comprised in the conjugate is characterised.

In another aspect, the methods involve a conjugate strand which comprises the target polypeptide. The polypeptide is attached to a polynucleotide flanking strand thereby forming the conjugate strand. The conjugate strand is contacted with a polynucleotide-handling protein. The conjugate strand is contacted with a nanopore. The polynucleotide-handling protein controls the movement of the conjugate strand with respect to the nanopore. One or more measurements characteristic of the polypeptide are taken as the conjugate moves with respect to the nanopore. In this manner, the target polypeptide which is comprised in the conjugate is characterised.

Accordingly, provided herein is a method of characterising a target polypeptide, comprising
contacting (i) a polynucleotide-polypeptide conjugate strand comprising the target polypeptide conjugated at each end of said target polypeptide to one or more polynucleotide flanking strands with (ii) a polynucleotide carrier strand, thereby forming a polynucleotide-polypeptide construct;
contacting the construct with a nanopore under conditions such that both the polynucleotide-polypeptide conjugate strand and the polynucleotide carrier strand co-translocate through the nanopore; and
taking one or more measurements characteristic of the polypeptide as the construct moves with respect to the nanopore, thereby characterising the target polypeptide.

In some embodiments, the one or more polynucleotide flanking strands are each independently complementary to a region of the polynucleotide carrier strand. In some embodiments, the one or more polynucleotide flanking strands are each independently at least partially hybridized to the polynucleotide carrier strand.

Also provided is a method of characterising a target polypeptide, comprising
contacting (i) a polynucleotide-polypeptide conjugate strand comprising a target polypeptide attached to a polynucleotide flanking strand with (ii) a polynucleotide-handling protein capable of controlling the movement of the polynucleotide flanking strand with respect to a nanopore; and
contacting the polynucleotide-polypeptide conjugate strand with a nanopore under conditions such that the polynucleotide-handling protein controls the movement of the polynucleotide-polypeptide conjugate strand with respect to the nanopore; and
taking one or more measurements characteristic of the polypeptide as the polynucleotide flanking strand and the target polypeptide co-translocate through the nanopore,
   thereby characterising the target polypeptide.

In some embodiments, prior to said method the polynucleotide flanking strand is at least partially hybridized to a polynucleotide carrier strand thereby forming a polynucleotide-polypeptide construct.

In some embodiments of the above methods, the polynucleotide-polypeptide conjugate strand comprises a plurality of target polypeptides.

In some embodiments, during said method the or each polypeptide is independently held in a linearized form.

In some embodiments, the or each target polypeptide independently has a length of from about 5 to about 1000 peptide units.

In some embodiments, said method comprises mechanically manipulating said construct, polynucleotide-polypeptide conjugate strand and/or polynucleotide carrier strand thereby moving said construct, polynucleotide-polypeptide conjugate strand and/or polynucleotide carrier strand with respect to the nanopore. In some embodiments, the construct, polynucleotide-polypeptide conjugate strand and/or polynucleotide carrier strand is moved by mechanical manipulation in a direction opposite to a potential applied across said nanopore. In some embodiments, said potential is a voltage potential applied across said nanopore.

In some embodiments, said method comprises contacting the construct with a polynucleotide-handling protein capable of controlling the movement of the one or more polynucleotide flanking strands and/or the polynucleotide carrier strand, and wherein the polynucleotide-handling protein controls the movement of the target polypeptide with respect to the nanopore.

In some embodiments, said method comprises contacting both polynucleotide-polypeptide conjugate strand and the polynucleotide carrier strand of the construct with a polynucleotide-handling protein capable of controlling the movement of the polynucleotide-polypeptide conjugate strand and/or the polynucleotide carrier strand, and wherein the polynucleotide-handling protein controls the movement of the construct with respect to the nanopore.

In some embodiments, said method comprises contacting the polynucleotide-polypeptide conjugate strand with a polynucleotide-handling protein capable of controlling the movement of the polynucleotide-polypeptide conjugate strand, and wherein the polynucleotide-handling protein controls the movement of the target polypeptide with respect to the nanopore.

In some embodiments, said method comprises contacting the polynucleotide carrier strand with a polynucleotide-handling protein capable of controlling the movement of the polynucleotide carrier strand, and wherein the polynucleotide-handling protein controls the movement of the target polypeptide with respect to the nanopore.

In some embodiments:
i) the polynucleotide-handling protein is located on the cis side of the nanopore and the polynucleotide-handling protein controls the movement of the construct, polynucleotide-polypeptide conjugate strand and/or polynucleotide carrier strand from the cis side of the nanopore to the trans side of the nanopore thereby controlling the movement of the target polypeptide from the cis side of the nanopore to the trans side of the nanopore; or
ii) the polynucleotide-handling protein is located on the trans side of the nanopore and the polynucleotide-handling protein controls the movement of the construct, polynucleotide-polypeptide conjugate strand and/or polynucleotide carrier strand from the trans side of the nanopore to the cis side of the nanopore thereby controlling the movement of the target polypeptide from the trans side of the nanopore to the cis side of the nanopore.

In some embodiments, the polynucleotide-handling protein is located on the cis side of the nanopore and the polynucleotide-handling protein controls the movement of the polynucleotide carrier strand from the cis side of the nanopore to the trans side of the nanopore, thereby controlling the movement of the target polypeptide through the nanopore.

In some embodiments the polynucleotide-handling protein is located on the trans side of the nanopore and the polynucleotide-handling protein controls the movement of the polynucleotide carrier strand from the trans side of the nanopore to the cis side of the nanopore, thereby controlling the movement of the target polypeptide through the nanopore.

In some embodiments:
i) the polynucleotide-handling protein is located on the cis side of the nanopore and the polynucleotide-handling protein controls the movement of the construct, polynucleotide-polypeptide conjugate strand and/or polynucleotide carrier strand from the trans side of the nanopore to the cis side of the nanopore thereby controlling the movement of the target polypeptide from the trans side of the nanopore to the cis side of the nanopore; or
ii) the polynucleotide-handling protein is located on the trans side of the nanopore and the polynucleotide-handling protein controls the movement of the construct, polynucleotide-polypeptide conjugate strand and/or polynucleotide carrier strand from the cis side of the nanopore to the trans side of the nanopore thereby controlling the movement of the target polypeptide from the cis side of the nanopore to the trans side of the nanopore.

In some embodiments, the polynucleotide-handling protein is located on the cis side of the nanopore and the polynucleotide-handling protein controls the movement of the polynucleotide carrier strand from the trans side of the nanopore to the cis side of the nanopore, thereby controlling the movement of the target polypeptide through the nanopore.

In some embodiments, the polynucleotide-handling protein is located on the trans side of the nanopore and the polynucleotide-handling protein controls the movement of the polynucleotide carrier strand from the cis side of the nanopore to the trans side of the nanopore, thereby controlling the movement of the target polypeptide through the nanopore.

In some embodiments, prior to the contacting of the construct with the nanopore the polynucleotide-handling protein is bound to the polynucleotide carrier strand in a region of the polynucleotide carrier strand that is spanned by a non-hybridised region of the polynucleotide flanking strand.

In some embodiments, the polynucleotide-handling protein is capable of remaining bound to the polynucleotide-polypeptide conjugate strand when the portion of the polynucleotide-polypeptide conjugate strand in contact with the active site of the polynucleotide-handling protein comprises the target polypeptide. In some embodiments, the polynucleotide-handling protein is modified to prevent it from disengaging from the construct, polynucleotide-polypeptide conjugate strand and/or polynucleotide carrier strand conjugate when the polynucleotide-handling protein contacts the target polypeptide. In some embodiments, the polynucleotide-handling protein is modified to wholly or partially close an opening existing in at least one conformation state of the unmodified protein through which a polynucleotide strand can unbind. In some embodiments, the polynucleotide-handling protein is or comprises a helicase, translocase or helicase-nuclease complex.

In some embodiments, the construct comprises a stalling moiety and prior to the translocation of the target polypeptide through the nanopore the polynucleotide-handling protein is positioned such that the stalling moiety is located between the polynucleotide-handling protein and the target polypeptide.

In some embodiments, one or more adapters and/or one or more tethers and/or one or more anchors are attached to the construct, polynucleotide-polypeptide conjugate strand and/or polynucleotide carrier strand. In some embodiments, the construct, polynucleotide-polypeptide conjugate strand and/or polynucleotide carrier strand comprises a blocking moiety attached via an optional linker, wherein the blocking moiety is incapable of translocating through the nanopore.

In some embodiments, the method comprises:
i) carrying out a method as described herein such that the target polypeptide translocates the nanopore in a first direction with respect to the nanopore;
ii) allowing the construct, polynucleotide-polypeptide conjugate strand and/or polynucleotide carrier strand to move in a direction opposite to the direction of movement with respect to the nanopore in step (i) such that the target polypeptide translocates the nanopore in a second direction which is opposite to the first direction;
iii) optionally allowing the construct, polynucleotide-polypeptide conjugate strand and/or polynucleotide carrier strand to move in the first direction such that the target polypeptide re-translocates the nanopore in the first direction;
iv) optionally repeating steps (ii) and (iii) to oscillate the polypeptide through the nanopore.

In some embodiments, the one or more measurements are characteristic of one or more characteristics of the target polypeptide selected from (i) the length of the target polypeptide, (ii) the identity of the target polypeptide, (iii) the sequence of the target polypeptide, (iv) the secondary structure of the target polypeptide and (v) whether or not the target polypeptide is modified.

In some embodiments, the nanopore is a protein nanopore, preferably a β-barrel protein nanopore.

Also provided herein is a system comprising
a construct comprising (i) a polynucleotide-polypeptide conjugate strand comprising a target polypeptide conjugated at each end of said target polypeptide to one or more polynucleotide flanking strands and (ii) a polynucleotide carrier strand;
a nanopore capable of co-translocating the polynucleotide-polypeptide conjugate strand and the polynucleotide flanking strand of the construct; and
a polynucleotide-handling protein.

Also provided herein is a kit comprising:
a nanopore;
a first polynucleotide comprising a reactive functional group for conjugating to a first end of a target polypeptide;
a second polynucleotide comprising a reactive functional group for conjugating to a second end of the target polypeptide; and
a polynucleotide-handling protein.

In some embodiments of the system and kit provided herein, the nanopore, construct and/or polynucleotide-handling protein are as defined herein.

Also provided are methods of characterising a target polynucleotide sequence. Unless implied otherwise by the context, the methods described herein that relate to characterisation of polypeptides can be applied analogously to the characterisation of target polynucleotide sequences, as described in more detail herein.

### BRIEF DESCRIPTION OF THE FIGURES

FIG. 1. Schematic showing a non-limiting example of an embodiment of the disclosed methods in which a polynucleotide-handling protein that moves on ssDNA at the cis side of a nanopore controls the movement of a construct comprising a polynucleotide-polypeptide conjugate strand as described herein hybridised to a polynucleotide carrier strand from the cis side of a nanopore to the trans side of the nanopore, thus allowing the polypeptide to be characterised as it moves with respect to the nanopore. As depicted the polynucleotide-handling protein is initially loaded on ssDNA opposite an optional bubble region (F). Both termini of the polynucleotide may be captured in a nanopore e.g. from the cis side of the membrane by the application of e.g. a positive voltage to the trans side of the membrane (i) as far as the polynucleotide-handing enzyme (ii), which destalls the polynucleotide-handling protein (if optional stalling chemistry is used) (iii) allowing it to translocate on ssDNA. The movement of the polynucleotide-handling protein along the polynucleotide section of the carrier strand feeds the construct into the pore; as the polynucleotide-handling protein moves along the polynucleotide (e.g. in 1 nucleotide fuel-driven steps) it feeds the construct into nanopore, and the peptide section passes through the nanopore allowing it to be characterised. Both ssDNA strands reanneal behind the enzyme, which as depicted translocates in a migrating bubble corresponding to polymer strand which spans the polynucleotide-handling protein.

A, peptide conjugated in dsDNA context; B, optional motor protein stalling chemistry (e.g. BNA, LNA, or RNA); C, optional motor protein stalling chemistry (e.g. spacer 18 or similar); D, polynucleotide handling enzyme; E, nanopore inserted in membrane; F, optional bubbled ssDNA, ssRNA or spacer chemistry opposite enzyme.

FIG. 2. Schematic showing a non-limiting example of an embodiment of the disclosed methods in which a polynucleotide-handling protein that moves on ssDNA at the cis side of a nanopore controls the movement of a construct comprising a polynucleotide-polypeptide conjugate strand as described herein hybridised to a polynucleotide carrier strand from the trans side of a nanopore to the cis side of the nanopore, thus allowing the polypeptide to be characterised as it moves with respect to the nanopore. As depicted, the polynucleotide-handling protein is initially loaded on ssDNA opposite an optional bubble region (F). Both termini of the polynucleotide are captured in a nanopore e.g. from the cis side of the membrane by the application of e.g. a positive voltage to the trans side of the membrane (i) as far as the polynucleotide-handing enzyme (ii), which removes the optional enzyme-stalling blocking moiety (if optional blocking moiety is used), (iii) allowing it to translocate on ssDNA. The movement of the polynucleotide-handling protein along the polynucleotide section of the carrier strand pulls the construct out of the pore (e.g. in the direction from the trans side to the cis side of the pore); as the polynucleotide-handling protein moves along the polynucleotide (e.g. in 1 nucleotide fuel-driven steps) it pulls the construct out of the nanopore, and the peptide section passes through the nanopore allowing it to be characterised. Both ssDNA strands reanneal behind the enzyme, which as depicted translocates in a migrating bubble, thereby controlling both strands of the conjugate out of the nanopore. All steps may be performed at positive bias on the trans side of the membrane except for (ii), which may be performed at zero potential or a low negative (in trans) bias.

A, optionally-bubbled ssDNA, ssRNA or spacer chemistry opposite enzyme; B, polynucleotide handling enzyme; C, optional enzyme-stalling blocking moiety (e.g. LNA, BNA or RNA); D, optional enzyme-stalling chemistry (e.g. spacer 18 or similar); E, peptide conjugated in dsDNA context; F, nanopore inserted in membrane.

FIG. 3. Schematic showing a non-limiting example of an embodiment of the disclosed methods in which a polynucleotide-handling protein at the cis side of a nanopore controls the movement of a construct comprising a polynucleotide-polypeptide conjugate strand as described herein hybridised to a polynucleotide carrier strand from the trans side of a nanopore to the cis side of the nanopore, thus allowing the polypeptide to be characterised as it moves with respect to the nanopore. As depicted, the polynucleotide-handling protein is initially loaded and optionally stalled on ssDNA opposite an optional bubble (D). An optional leader (A) may be present on the construct in order to facilitate threading of the construct through the nanopore. Both termini of the polynucleotide are captured in a nanopore (e.g. via the optional leader) e.g. from the cis side of the membrane by the application of e.g. a positive voltage to the trans side of the membrane (i) as far as the polynucleotide-handing enzyme (ii), after which the enzyme is pushed backwards to the optional blocking moiety (F), (iii) then allowing it to translocate on ssDNA (iv). The movement of the polynucleotide-handling protein pulls the construct out of the pore (e.g. in the direction from the trans side to the cis side of the pore); as the polynucleotide-handling protein moves along the polynucleotide (e.g. in 1 nucleotide fuel-driven steps) it pulls the construct out of the nanopore, and the peptide section passes through the nanopore allowing it to be characterised. Both ssDNA strands reanneal behind the enzyme, which as depicted translocates in a migrating bubble, thereby controlling both strands of the conjugate out of the nanopore. The enzyme may be pushed back to an earlier position at any point via the force acting on the DNA, which resets the cycle. The polynucleotide-handling protein can then again control the movement of the construct with respect to the nanopore allowing the target polypeptide to be repeatedly “flossed” through the nanopore.

A, optional leader section; B, optional C3 section for stalling enzyme; C, polynucleotide handling enzyme; D, optionally-bubbled ssDNA, ssRNA or spacer chemistry opposite enzyme; E, peptide conjugated in dsDNA context; F, optional back-blocker moiety to prevent dissociation of enzyme; G, nanopore inserted in membrane.

FIG. 4. Schematic showing a non-limiting example of an embodiment of the disclosed methods in which a polynucleotide-handling protein at the cis side of a nanopore controls the movement of a construct comprising a polynucleotide-polypeptide conjugate strand as described herein hybridised to a polynucleotide carrier strand from the cis side of a nanopore to the trans side of the nanopore, thus allowing the polypeptide to be characterised as it moves with respect to the nanopore. The polynucleotide-handling protein engages with both strands of the construct (e.g. with the polynucleotide-polypeptide conjugate strand and with the carrier strand) and may actively control the movement of either or both strands. The movement of the construct and the characterisation of the target polypeptide is as discussed for FIG. 1.

FIG. 5. Schematic showing a non-limiting example of an embodiment of the disclosed methods in which a polynucleotide-handling protein at the cis side of a nanopore controls the movement of a construct comprising a polynucleotide-polypeptide conjugate strand as described herein hybridised to a polynucleotide carrier strand from the trans side of a nanopore to the cis side of the nanopore, thus allowing the polypeptide to be characterised as it moves with respect to the nanopore. The polynucleotide-handling protein engages with both strands of the construct (e.g. with the polynucleotide-polypeptide conjugate strand and with the carrier strand) and may actively control the movement of either or both strands. The movement of the construct and the characterisation of the target polypeptide is as discussed for FIG. 2.

FIG. 6. Schematic showing further non-limiting examples of embodiments of the disclosed methods. A: A target polypeptide is comprised in a polynucleotide-polypeptide conjugate strand by being attached to a polynucleotide flanking strand. The polynucleotide-polypeptide conjugate strand is contacted with a polynucleotide-handling protein thus allowing the polypeptide to be characterised as it moves with respect to the nanopore. As depicted the movement scheme is as in FIG. 1 however those skilled in the art will appreciate that the exact movement scheme is not limited and movement scheme such as those described herein and particularly in each of FIGS. 1 to 5 are also compatible with this embodiment, and are hereby specifically disclosed. B: The polynucleotide-polypeptide conjugate strand of panel A may be optionally contacted with a polynucleotide carrier strand in order to form a construct as depicted. The construct is contacted with a polynucleotide-handling protein thus allowing the polypeptide to be characterised as it moves with respect to the nanopore. As depicted the movement scheme is as in FIG. 4 however those skilled in the art will appreciate that the exact movement scheme is not limited and movement scheme such as those described herein and particularly in each of FIGS. 1 to 5 are also compatible with this embodiment, and are hereby specifically disclosed. C: The polynucleotide-polypeptide conjugate strand of panel A may be optionally contacted with a polynucleotide carrier strand in order to form a construct as depicted; with the carrier strand optionally being excluded from the nanopore during the translocation of the construct through the nanopore. As depicted the movement scheme is as in FIG. 1 however those skilled in the art will appreciate that the exact movement scheme is not limited and movement scheme such as those described herein and particularly in each of FIGS. 1 to 5 are also compatible with this embodiment, and are hereby specifically disclosed.

FIG. 7. Schematic showing a further non-limiting example of a method for repetitive, controlled movement of polynucleotide-polypeptide conjugate from trans to cis, in which movement control is via a polynucleotide handling enzyme that moves on ssDNA. Enzyme is initially loaded and stalled on an ssDNA overhang. The method is performed under conditions where multiple enzyme may load on the overhang. Both termini of the polynucleotide are captured in a nanopore (i) as far as the polynucleotide-handing enzyme (ii), after which the enzyme controls movement of the conjugate out of the nanopore as far as the peptide (iii). The enzyme may then dissociate from the polynucleotide, at which point the position of the conjugate in the nanopore drops to a second enzyme loaded at an earlier position on the DNA, which resets the cycle.

A, polynucleotide-handling enzyme loaded on ssDNA overhang; B, peptide conjugated in dsDNA context; C, nanopore inserted in membrane; D, second polynucleotide-handling enzyme binding to ssDNA overhang while first is translocating.

FIG. 8. Schematic showing a further non-limiting example of a method for repetitive, controlled movement of polynucleotide-polypeptide conjugate from trans to cis, in which movement control is via a polynucleotide handling enzyme that moves on ssDNA. Scheme is identical to FIG. 7 except that the enzyme is loaded on a strand that is continuously ssDNA, except for a spacer moiety (A), which restricts movement of the enzyme and causes the enzyme to disengage from DNA.

FIG. 9. Schematic showing further non-limiting examples of schemes in which a polypeptide is characterised using a nanopore. The polypeptide in each example is conjugated between two internal groups on a polynucleotide. A: Polynucleotide-handling enzyme controls polynucleotide-polypeptide conjugate out of nanopore. Polypeptide is attached to two internal points on a ssDNA oligonucleotide and either (i) ssDNA and peptide or (ii) dsDNA and peptide co-translocate through the nanopore. B: Polynucleotide-handling enzyme controls polynucleotide-polypeptide conjugate into nanopore. Polypeptide is attached to a single point on a ssDNA oligonucleotide and either (i) ssDNA and peptide or (ii) dsDNA and peptide co-translocate through the nanopore. C: Polynucleotide-handling enzyme controls polynucleotide-polypeptide conjugate into nanopore. Polypeptide is attached to two internal points on a ssDNA oligonucleotide and either (i) ssDNA and peptide or (ii) dsDNA and peptide co-translocate through the nanopore. In case (ii) in each scheme shown, the polynucleotide-handling enzyme may translocate on either DNA strand.

FIG. 10. Assembly method for polynucleotide-polypeptide constructs. In a first click reaction (step (i)), a hairpin DNA (DNA1) bearing 3′ TCO group is reacted with a polypeptide bearing N-terminal azide and C-terminal methyltetrazine groups. A second click reaction (step (ii)) to DNA2 is then performed; DNA2 bears a 5′ BCN group and 3′ biotin group. To generate the final construct (A), monovalent traptavidin is added (step (iii)). The distance x, defined as the distance in base-pairs between the monovalent traptavidin and peptide (and excluding any intervening linker chemistries), is varied in the experiments described in Example 1. The “carrier” strand (opposite the peptide) in this example has a length of 6 nucleotides. The hairpin end is captured in the nanopore was shown in (A).

FIG. 11. Example current-time traces showing capture of a polynucleotide-polypeptide conjugate, as described in Example 1. The peptide sequence used throughout is N-GGSGXXSGSG-C: in trace (A), XX=DD; in (B), XX=RR; and in (C), XX=YY. Each traces shows an initial phase of open pore current (i) followed by a drop to a lower level (ii) owing to capture of the conjugate. The normalised current (I/IO; FIG. 12) is then scored for each capture as the median current of level (ii) divided by the median current of level (i).

FIG. 12. Histograms of normalised currents for three peptides (sequence N-GGSGXXSGSG-C, where in (A) XX=DD; in (B), XX=RR; and in (C), XX=YY). Normalised current is defined as per FIG. 11.

FIG. 13. Plots of normalised current vs. distance for a series of polynucleotide-polypeptide conjugates in which the polypeptide bears the sequence N-GGSGXXSGSG-C, and the central two residues are mutated to DD, RR or YY. The normalised current (FIG. 11) is plotted as a function of the distance, in base pairs, between monovalent traptavidin and peptide. The dsDNA level, shown as a dashed line, was determined from a double-stranded DNA control.

FIG. 14. Example current-time traces showing capture of a polynucleotide-polypeptide conjugate, with movement of the conjugate out of the nanopore controlled by a helicase, as described in Example 2. The peptide sequence used throughout is N-GGSGXXSGSG-C: in trace (A), XX=DD; in (B), XX=RR; and in (C), XX=YY. Each trace shows an initial phase of open pore current (i) followed by a drop to a lower level marked by (ii) owing to capture of the conjugate. Each example shows a repetitive pattern indicating repetitive movement of the conjugate through the nanopore. At (iii) the helicase controlling the movement dissociates from the conjugate and there is no second helicase bound, so the current trace returns to the open pore level (i).

FIG. 15. Example current-time traces showing capture of a polynucleotide-polypeptide conjugate, with the conjugate bound by a helicase, but in the absence of ATP, as described in Example 2. The peptide sequence used is N-GGSGDDSGSG-C. The trace shows an initial phase of open pore current (i) followed by a drop to a lower level marked by (ii) owing to capture of the conjugate. Unlike FIG. 14, no repetitive motion is seen, demonstrating that movement of the helicase is ATP-dependent. At (iii) the helicase controlling the movement dissociates from the conjugate and there is no second helicase bound, so the current trace returns to the open pore level (i).

FIG. 16. Polynucleotide-polypeptide construct used in Example 3: a. leader; b. tether; c. hairpin; d. DNA 1 oligo; e. DNA 2 oligo; f. peptide; g. DNA 3 oligo; x. ssDNA overhang for enzyme loading; y. helicase, with arrow indicating direction of helicase movement; Circle=click chemistry group; Vertical line=ligation site.

FIG. 17. Current-time traces showing capture of a polynucleotide-polypeptide conjugate, with movement of the conjugate out of the nanopore controlled by a helicase, as described in Example 3. The tested peptide sequences were RSDSGQQARY (FIG. 17A, 2D), GGSGSSSGSG (FIG. 17B, 17E), and EAIYAAPFAKKK (FIG. 17C, 17F). Each trace A, Band C shows an initial phase of open pore current (i) followed by a drop to a lower level (ii) owing to capture of the conjugate. Each trace A, B and C further shows a repetitive pattern indicating repetitive movement of the conjugate through the nanopore. At (iii) the helicase controlling the movement dissociates from the conjugate and there is no further helicase bound, so the current trace returns to the open pore current level. Examples of single reads for each peptide are shown in D, E and F. Single reads exhibit a single-stranded DNA phase (iv), followed by a dip in current caused by the peptide (v). The length of block (iv) depends on where along the single-stranded overhang the enzyme is bound when the conjugate enters the nanopore. The 100 pA current level is shown as a dashed line to highlight the difference in the peptide dip amplitude among the different sequences tested.

FIG. 18. Current-time traces from Example 3, showing single reads of polynucleotide-polypeptide conjugates comprising peptides of varying charge. Movement of the conjugate out of the nanopore is controlled by a helicase, as described in Example 3. The peptide sequences are SRRRRRRRRS (A), HDSGYEVHHQK (B), and SEEEEEEEES (C), with charges of +8, −2, and −8, respectively. Single reads exhibit a single stranded DNA phase (iv), followed by a dip in current caused by the peptide (v). The length of block (iv) depends on where along the single-stranded overhang the enzyme is bound when the conjugate enters the nanopore. The 0 pA current level is shown as a dashed line to highlight the difference in peptide block amplitude among the different sequences tested.

FIG. 19. Polynucleotide-polypeptide construct used in Example 4. a. leader; b. tether; c. hairpin adapter; d. DNA 1 oligo; e. DNA 2 oligo; f. peptide; g. DNA 3 oligo; x. stall; y. helicase, with arrow indicating direction of helicase movement; z. bubble; Circle=click chemistry group; Vertical line=ligation site;

FIG. 20. Current-time traces from Example 4, showing capture of a polynucleotide-polypeptide conjugate, with movement of the conjugate into the nanopore controlled by a Dda helicase, as described in Example 4. The peptide sequence is HDSGDEVHHQK, a fragment of amyloid beta protein. Three examples traces are shown, demonstrating signal reproducibility. Each trace shows an initial phase of open pore current (i) followed by a drop to a lower level (ii), owing to capture of the conjugate and stalling of the helicase. Once the enzyme is de-stalled by the electrophoretic force, the signal transitions to a dsDNA level (iii) which is followed by a dip in the signal caused by the peptide (iv). Once the helicase has moved the peptide past the constriction, the signal returns to the dsDNA level (iii).

FIG. 21. Polynucleotide-polypeptide construct used in Example 5. a. DNA 1 oligo; b. DNA 2 oligo; c. peptide; d. DNA 3 oligo; e. DNA 4 oligo; f. sequencing adapter; x. stall; y. helicase, with arrow indicating direction of helicase movement; Circle=click chemistry group, located within DNA 2 and DNA 4 oligos; Vertical line=ligation site;

FIG. 22. A1: current-time trace of enzymatically-controlled movement of the polypeptide-polynucleotide conjugate, including co-translocation of DNA-peptide, as described in Example 5. A2: zoom-in on highlighted section in A1 trace. B1: current-time trace for control experiment in which peptide translocates nanopore alone. B2: zoom-in on highlighted section in B1 trace.

Each trace shows an initial phase of open pore current (i), which, upon analyte capture, drops to a section of adapter sequence (ii) followed by a spike in current caused by the peptide (iii). The peptide is flanked by oligos containing 40 dT bases, which create a flat section in the signal on either side of the peptide (iv).

FIG. 23. Polynucleotide-polypeptide construct used in Example 6. a. DNA 1 oligo; b. DNA 2 oligo, containing internal click chemistry group, and showing DNA ‘flap’ alongside peptide; c. peptide; d. DNA 3 oligo; e. DNA 4 oligo; f. sequencing adapter; x. stall; y. helicase, with arrow indicating direction of helicase movement; Circle=click chemistry group; Vertical line=ligation site;

FIG. 24. A1: current-time trace of enzymatically-controlled movement of the polypeptide-polynucleotide conjugate, including co-translocation of DNA flap and peptide, as described in Example 6. A2: zoom-in on highlighted section in A1 trace.

Each trace shows an initial phase of open pore current (i), which, upon analyte capture, drops to a section of adapter sequence (ii) followed by a spike in current caused by the peptide (iii). The peptide is flanked by oligos containing 40 dT bases, which create a flat section in the signal on either side of the peptide (iv).

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a motor protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### DEFINITIONS

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention.

Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, atemporal duration, and the like, is meant to encompass variations of ±20% or +10%, more preferably +5%, even more preferably +1%, and still more preferably +0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as 3-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Disclosed Methods

The disclosure relates to methods of characterising polypeptides. The polypeptides are characterised as they co-translocate through a nanopore together with one or more polynucleotide strands, such as one or more polynucleotide flanking strand and/or carrier strands as described in more detail herein. The polypeptide and polynucleotide strands together may be referred to as a construct.

The methods exploit the ability of many nanopores to simultaneously accommodate multiple polymer strands. The co-translocation of the polypeptide strand and one or more polynucleotide strands has advantages as discussed in more detail herein.

In contrast to methods which seek to control the movement of a polypeptide with respect to a nanopore using a polypeptide-handling enzyme, certain embodiments of the present disclosure relate to methods which involve controlling the movement of a polypeptide with respect to a nanopore using a polynucleotide-handling enzyme. Other embodiments of the present disclosure do not require the use an enzyme to control the movement of the construct.

Accordingly, in one aspect is provided herein a method of characterising a target polypeptide, comprising
contacting (i) a polynucleotide-polypeptide conjugate strand comprising a target polypeptide conjugated at each end of said target polypeptide to one or more polynucleotide flanking strands with (ii) a polynucleotide carrier strand, thereby forming a polynucleotide-polypeptide construct;
contacting the construct with a nanopore under conditions such that both the polynucleotide-polypeptide conjugate strand and the polynucleotide carrier strand co-translocate through the nanopore; and
taking one or more measurements characteristic of the polypeptide as the construct moves with respect to the nanopore, thereby characterising the target polypeptide.

In a related aspect, provided is a method of characterising a target polypeptide, comprising
contacting (i) a polynucleotide-polypeptide conjugate strand comprising a target polypeptide conjugated at each end of said target polypeptide to one or more polynucleotide flanking strands with (ii) a polynucleotide carrier strand, thereby forming a polynucleotide-polypeptide construct;
controlling the movement of the construct through a nanopore such that both the polynucleotide-polypeptide conjugate strand and the polynucleotide carrier strand co-translocate through the nanopore; and
taking one or more measurements characteristic of the polypeptide as the construct moves with respect to the nanopore,
   thereby characterising the target polypeptide.

In another related aspect of the present disclosure is provided a method of characterising a target polypeptide, comprising
contacting (i) a polynucleotide-polypeptide conjugate strand comprising a target polypeptide attached to a polynucleotide flanking strand with (ii) a polynucleotide-handling protein capable of controlling the movement of the polynucleotide flanking strand with respect to a nanopore; and
contacting the polynucleotide-polypeptide conjugate strand with a nanopore under conditions such that the polynucleotide-handling protein controls the movement of the polynucleotide-polypeptide conjugate strand with respect to the nanopore; and
taking one or more measurements characteristic of the polypeptide as the polynucleotide flanking strand and the target polypeptide co-translocate through the nanopore,
   thereby characterising the target polypeptide.

In a related aspect, provided is a method of characterising a target polypeptide, comprising
contacting (i) a polynucleotide-polypeptide conjugate strand comprising a target polypeptide attached to a polynucleotide flanking strand with (ii) a polynucleotide-handling protein;
controlling the movement of the polynucleotide flanking strand with respect to a nanopore using the polynucleotide-handling protein; and
contacting the polynucleotide-polypeptide conjugate strand with a nanopore under conditions such that the polynucleotide-handling protein controls the movement of the polynucleotide-polypeptide conjugate strand with respect to the nanopore; and
taking one or more measurements characteristic of the polypeptide as the polynucleotide flanking strand and the target polypeptide co-translocate through the nanopore,
   thereby characterising the target polypeptide.

It will be apparent to those skilled in the art that a polynucleotide-handling protein is not essential in the methods provided herein, although in some preferred embodiments a polynucleotide-handling protein is used to control the movement of the construct, polynucleotide-polypeptide conjugate strand and/or carrier strand as described herein. Accordingly, in some embodiments, e.g. in some embodiments which do not necessarily involve a polynucleotide-handling protein, provided herein is a method of characterising a target polypeptide, comprising
contacting (i) a polynucleotide-polypeptide conjugate strand comprising a target polypeptide attached to a polynucleotide flanking strand with (ii) a nanopore;
controlling the movement of the polynucleotide-polypeptide conjugate strand with respect to the nanopore; and
taking one or more measurements characteristic of the polypeptide as the polynucleotide flanking strand and the target polypeptide co-translocate through the nanopore,
   thereby characterising the target polypeptide.

The above methods may be referred to herein as disclosed methods. For avoidance of doubt, herein embodiments of the present disclosure are described in relation to the disclosed methods for brevity. Unless required otherwise by the context, such embodiments are expressly disclosed in relation to and as preferred features of each of the disclosed methods above.

Any suitable polypeptide can be characterised using the methods disclosed herein. In some embodiments the target polypeptide is a protein or naturally occurring polypeptide. In some embodiments the target polypeptide is a portion of a protein or naturally occurring polypeptide, such as may be obtained by nuclease digestion of a protein or naturally occurring polypeptide. In some embodiments the polypeptide is a synthetic polypeptide. Polypeptides which can be characterised in accordance with the disclosed methods are described in more detail herein.

Any suitable polynucleotide can be used in forming the polynucleotide-polypeptide conjugate strand for use in the methods disclosed herein. The polynucleotide strand(s) which are attached to the polypeptide strand in the polynucleotide-polypeptide conjugate strand may be referred to as flanking strands. Flanking strands are described in more detail herein.

In some embodiments the or each polynucleotide flanking strand has a length at least as long as a portion of the target polypeptide to be characterised. In some embodiments the or each polynucleotide flanking strand has a greater length than the portion of the target polypeptide to be characterised. In some embodiments the or each polynucleotide flanking strand has a length which is shorter than the portion of the target polypeptide to be characterised. Polynucleotides suitable for use in the disclosed methods are disclosed in more detail herein.

In some embodiments, the or each polynucleotide-polypeptide conjugate strand is complexed with a polynucleotide carrier strand. In some embodiments the polynucleotide flanking strands of the polynucleotide-polypeptide conjugate strand are each independently complementary to a region of the polynucleotide carrier strand. In some embodiments the polynucleotide flanking strands of the polynucleotide-polypeptide conjugate strand are each independently at least 60%, at least 70%, at least 80%, at least 90%, at least 95%, at least 96%, at least 97%, at least 98%, at least 99%, at least 99.5% complementary to the corresponding region of the polynucleotide carrier strand.

In some embodiments the one or more polynucleotide flanking strands are each independently at least partially hybridized to the polynucleotide carrier strand. Each flanking strand may be independently at least 60%, at least 70%, at least 80%, at least 90%, at least 95%, at least 96%, at least 97%, at least 98%, at least 99%, at least 99.5% hybridised to the complementary strand over the length of the flanking strand. For example, in some embodiments in some embodiments the one or more polynucleotide flanking strands are each independently hybridised to the polynucleotide carrier strand over a length of at least 5, e.g. at least 10, e.g. at least 20, e.g. at least 30, e.g. at least 40, e.g. at least 50, e.g. at least 60, e.g. at least 80, e.g. at least 100, such as at least 150, e.g. at least 200, e.g. at least 500, or more nucleotides. Complementary strands as described herein may associate via e.g. hydrogen bonding (e.g. base pairing) and thus hybridise together. Those skilled in the art will appreciate that the strength of the hybridisation between the polynucleotide carrier strand and the polynucleotide flanking strand of the polynucleotide-polypeptide conjugate strand can be controlled by controlling i.a. the length of the hybridised region and the degree of complementarity. Shorter flanking strands may be used to decrease the hybridisation force between the flanking strand and the carrier strand of the polynucleotide-polypeptide conjugate strand. Longer strands may be used to increase the hybridisation force between the flanking strand and the carrier strand of the polynucleotide-polypeptide conjugate strand. Increasing the degree of complementarity may be used to increase the hybridisation force between the flanking strand and the carrier strand of the polynucleotide-polypeptide conjugate strand.

In some embodiments the polynucleotide-polypeptide conjugate strand or the carrier strand may overhang the other strand of the construct. For example, in some embodiments the polynucleotide-polypeptide conjugate strand overhangs the carrier strand at one end of the carrier strand. For example, the 3′ end of the polynucleotide-polypeptide conjugate strand may overhang the 5′ end of the carrier strand. In other embodiments, the carrier strand may overhang the polynucleotide-polypeptide conjugate strand at one end of the polynucleotide-polypeptide conjugate strand. For example, the 3′ end of the carrier strand may overhang the 5′ end of the polynucleotide-polypeptide conjugate strand. Of course the overhang may alternatively be present at another end of the construct, for example, the 5′ end of the polynucleotide-polypeptide conjugate strand may overhang the 3′ end of the carrier strand; or the 5′ end of the carrier strand may overhang the 3′ end of the polynucleotide-polypeptide conjugate strand. In some embodiments an overhang may be present at each end of the construct. In some embodiments, the overhang provides a loading site for the polynucleotide-handling protein.

In the disclosed methods, the target polypeptide can be conjugated to the polynucleotide using any suitable means. Some exemplary means are described in more detail herein.

In some embodiments, as discussed in more detail herein, the conjugate formed in the disclosed methods is contacted with a polynucleotide-handling protein. The polynucleotide-handling protein is typically capable of controlling the movement of the polynucleotide flanking strand and/or a polynucleotide carrier strand if present with respect to a nanopore. Exemplary polynucleotide-handling proteins are described in more detail herein.

When present, the polynucleotide-handling protein controls the movement of a polynucleotide strand with respect to a nanopore. As discussed in more detail herein, the polynucleotide-handling protein may control the movement of the polynucleotide flanking strand with respect to a nanopore. The polynucleotide-handling protein may control the movement of a polynucleotide carrier strand with respect to the nanopore. The polynucleotide-handling protein may control the movement of both the polynucleotide flanking strand and a polynucleotide carrier strand with respect to the nanopore. Thus, the polynucleotide-handling protein controls the movement of the construct with respect to the nanopore. Any suitable nanopore can be used in the disclosed methods. Nanopores suitable for use in the disclosed methods are described in more detail herein.

The disclosed methods comprise taking one or more measurements characteristic of the polypeptide as the or each polypeptide portion of the polynucleotide-polypeptide conjugate strand moves with respect to the nanopore. The one or more measurements can be any suitable measurements. Typically, the one or more measurements are electrical measurements, e.g. current measurements, and/or are one or more optical measurements. Apparatuses for recording suitable measurements, and the information that such measurements can provide, are described in more detail herein.

Certain disclosed methods can also be used to characterise a target polynucleotide, and features of the disclosed methods can be generally applied to such methods unless implied otherwise by the context. Certain methods of characterising target polynucleotides are described in more detail herein.

### Characterising a Target Polypeptide

Described herein are methods of characterising a target polypeptide, comprising forming a construct comprising a first strand comprising a target polypeptide and a second strand comprising a polynucleotide, moving the construct with respect to a nanopore under conditions such that the first and second strands of the construct co-translocate through the nanopore, and taking one or more measurements characteristic of the polypeptide as the construct moves with respect to the nanopore. The methods described herein may comprise the step of controlling the movement of the movement of the construct through the nanopore.

As disclosed herein, a polynucleotide can be used to control the movement of a target polypeptide with respect to a nanopore. Because the polynucleotide is conjugated to the polypeptide in the conjugate, the movement of the polynucleotide drives the movement of the polypeptide. The polypeptide portion of the polynucleotide-polypeptide conjugate strand is flanked by one or more polynucleotide flanking strands. The polynucleotide-polypeptide conjugate strand may be hybridised to a polynucleotide carrier strand.

The methods provided herein thus involve the co-translocation of (i) the target polypeptide and (ii) a polynucleotide strand through the nanopore. In some embodiments the polynucleotide strand that co-translocates through the nanopore is a polynucleotide flanking strand as described herein. In some embodiments the polynucleotide strand that co-translocates through the nanopore is a polynucleotide carrier strand as described herein.

As used herein, the term “co-translocate” means to translocate together and simultaneously. Thus, with respect to the methods described herein, to co-translocate through a nanopore means to translocate together and simultaneously through a nanopore.

In the methods described herein, a target polypeptide and a polynucleotide strand co-translocate through a nanopore. Thus, the target polypeptide and the polynucleotide strand translocate through the nanopore together and simultaneously. Such co-translocation of two strands necessarily requires that the two strands pass through the nanopore alongside one another (side by side), in a double-stranded configuration. Thus, by way of further example, in some embodiments of the methods described herein, a target polypeptide and a polynucleotide strand that co-translocate through a nanopore may do so in the form of a double-stranded polypeptide-polynucleotide chimera.

The above can be contrasted with prior art methods such as described in WO 2021/111125 and WO 2021/133168. In such prior art methods, a single-stranded polypeptide-polynucleotide conjugate is moved through a nanopore using a polynucleotide-handling enzyme, such that polypeptide and polynucleotide portions of the conjugate move through (i.e. translocate) the nanopore one after another in a linear fashion, with no double-stranded polypeptide-polynucleotide ever moving through the pore. Such linear translocation of a polypeptide and polynucleotide one after the other contrasts with the “in parallel” movement of a target polypeptide and polynucleotide strand provided by the methods of the present invention disclosed herein.

The simultaneous co-translocation of the target polypeptide and a polynucleotide strand means that at the point of measurement the analyte (e.g. the construct) that moves through the pore is necessarily double-stranded. One strand comprises a polynucleotide. The other strand comprises the target polypeptide. Thus, the term “double-stranded” as used herein embraces a polynucleotide strand in parallel with a polypeptide strand. This is associated with advantages compared to methods for characterising polypeptides known in the art.

By way of example, target polypeptides are typically substantially uncharged or have low net charge and/or charge density, and/or are irregularly charged. In other words, charge distribution in a target polypeptide is typically irregularly distributed along the length of a target polypeptide. As set out in Table 1 above, some amino acids which are comprised in target polypeptides are polar, and some are non-polar. Some are positively or negatively charged under physiological conditions, others are uncharged under physiological conditions but may be charged under the conditions under which methods such as those disclosed herein are carried out, and yet others are uncharged under all relevant conditions. The distribution of amino acids in the target polypeptide is a function of the exact analyte being characterised in the disclosed methods and thus may not be known by the user in advance.

In known methods of polypeptide analysis which rely on electrophoretic movement of polypeptides through a nanopore, this irregular charge along a target polypeptide may present difficulties, because the electrophoretic force acting on the polypeptide will vary as the polypeptide strand moves through the nanopore. In consequence, the rate of movement of the polypeptide through the nanopore may be unpredictable, which hampers accurate characterisation. For example, it may be difficult to distinguish two identical amino acids which move quickly through a pore from one amino acid which moves more slowly. The low average charge density of target polypeptides is one reason that methods which may comprise detection or characterisation of polynucleotides and analogues thereof (such as PNA; peptide nucleic acid) are typically unsuitable for the accurate characterisation of target polypeptides. Accordingly, there is a need for methods which do not rely on the varying charge of the polypeptide to determine the movement of the polypeptide through the pore.

A further issue that arises in known methods of polypeptide analysis is that the target polypeptide typically has an irregular 3D structure. For example, proteins are known to fold to adopt 3D structures which may be associated with their biological function. The presence of 3D structure (e.g. secondary or tertiary structure) in a target polypeptide may hamper its characterisation using a nanopore in known methods which rely on the single-stranded translocation of a target polypeptide through the pore. This is because different portions of a folded target polypeptide will require different degrees of force to unfold them in order to translocate in this manner. The consequence of this is that the movement of the polypeptide through the pore can be irregular, for example with some portions moving more quickly through the pore compared to other portions. This can hamper accurate characterisation. Furthermore, it may not be possible to unfold proteins using a nanopore in this manner. Thus, methods which involve merely decorating a double-stranded polynucleotide with a folded protein and translocating the construct through a nanopore (e.g. a solid state nanopore) are typically incapable of providing detailed information regarding the protein, such as its sequence.

A still further issue that may arise in some known methods of characterising polypeptides using nanopores is that motor proteins which may be used to control the movement of such polypeptides may sometimes be inefficient at precisely controlling the movement of long polypeptide strands, even though they may effectively translocate on such strands. For example, when used to control the movement of very long polypeptide strands (e.g. in the form of polynucleotide-polypeptide conjugates) a motor protein may slip on the polypeptide portion of the strand when it moves through the nanopore. Slippage is problematic because it can lead to inaccurate characterisation of the polypeptide.

The present methods address some or all of these issues. As noted above, the simultaneous co-translocation of the target polypeptide and a polynucleotide strand means that at the point of measurement the analyte that moves through the pore is necessarily double-stranded. In some embodiments this leads to some or all of the following advantages.

First, a polynucleotide strand that co-translocates the nanopore with the target polypeptide will typically have a regular charge density and will thus exert a regular electrophoretic force on the construct as it moves with respect to the nanopore. The force exerted by movement of a polynucleotide through a nanopore is well understood and can be accurately modelled. The co-translocated polynucleotide may thus drive the predictable and consistent movement of a target polypeptide through a pore, facilitating characterisation of the polypeptide.

Second, a polynucleotide strand that co-translocates the nanopore with the target polypeptide can be chosen or configured in order to effectively linearize the target polypeptide. For example, the target polypeptide may be attached at each end of the analyte to a polynucleotide flanking strand and the polynucleotide flanking strand may be hybridised to a polynucleotide carrier strand. The hybridisation of the flanking strands prevents the folding of the polypeptide. The flanking strands may be designed to stretch the target polypeptide to a desired extent according to the parameters of the method as operated by the used. Because folding of the target polypeptide can be reduced or abolished, the movement of the target polypeptide through the nanopore is typically more regular.

Third, a polynucleotide-handling protein may be contacted with the polynucleotide-polypeptide conjugate strand and/or with a polynucleotide carrier strand and used to control the construct with respect to the nanopore. In embodiments wherein the polynucleotide-handling protein is used to control the movement of a polynucleotide strand in order to control the movement of the construct the length of the target polypeptide is not particularly limited, because the polynucleotide-handling protein progressively processes the polynucleotide strand that co-translocates the pore with the polypeptide strand even whilst the polypeptide strand is moving through the nanopore.

Accordingly, the methods provided herein allow for some or all of improved ability to detect target polypeptides; improved accuracy in polypeptide characterisation, improved reproducibility of polypeptide data, improved (increased) polypeptide read lengths, and decreased slippage.

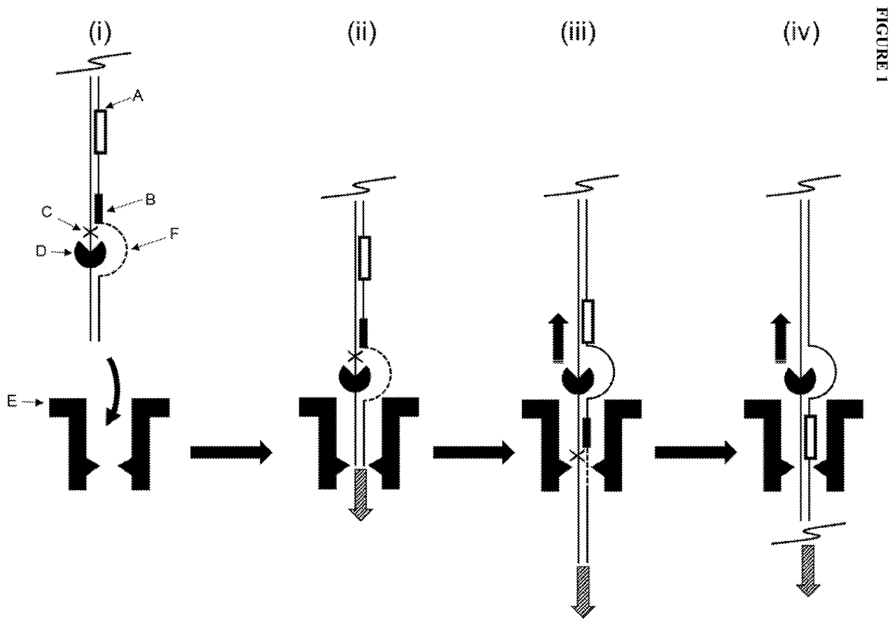

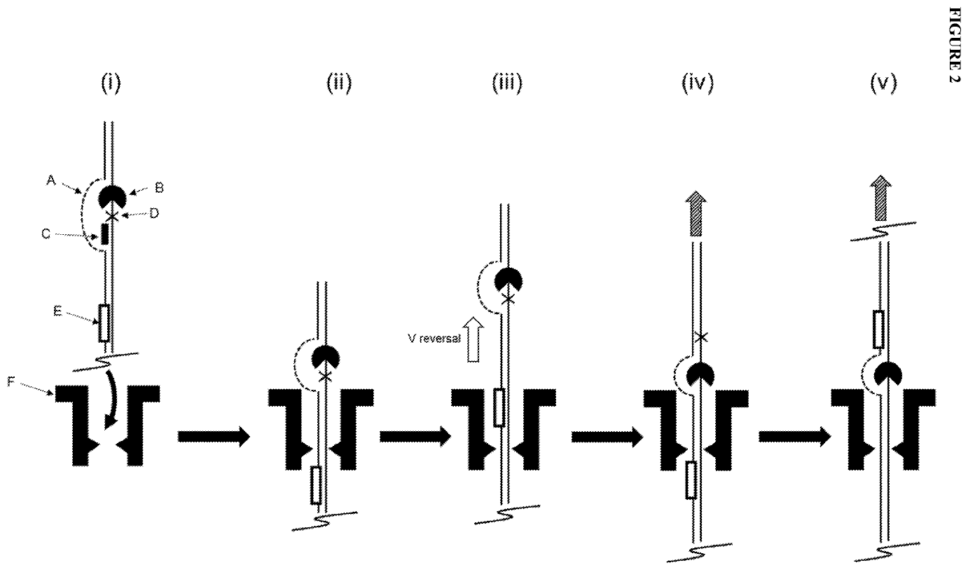

The methods of the present invention can be understood by reference to FIG. 1, which illustrates one non-limiting example of the disclosed method.

A construct may comprise a polynucleotide-polypeptide conjugate strand which comprises a target polypeptide flanked by one or more polynucleotide flanking strands. The construct may in some embodiments be contacted with a polynucleotide-handling protein such that the construct threads the nanopore. Optionally, a further polymer such as a leader (described herein) may be used to facilitate the threading of the polypeptide through the nanopore. Such use is within the scope of the disclosed methods, however this is not essential. Optionally, a spacer and/or stall may be used to restrain the movement of the polynucleotide-handling protein prior to the operation of the methods by the user (depicted in FIG. 1). This is described herein and is within the scope of the disclosed methods, but is not essential.

In some embodiments the polynucleotide-handling protein may process the polynucleotide carrier strand. As the polynucleotide-handling protein processes the polynucleotide carrier strand, the construct is passed through the nanopore because the carrier strand is hybridised to the polynucleotide-polypeptide conjugate strand (e.g. to the flanking strand portions of the polynucleotide-polypeptide conjugate strand). Accordingly, the polypeptide is passed through the nanopore. As the polypeptide is passed through the nanopore it is characterised.

In some embodiments the polynucleotide-handling protein may process the polynucleotide-polypeptide conjugate strand (e.g. by processing the polynucleotide flanking strand(s)). As the polynucleotide-handling protein processes the polynucleotide flanking strand, the construct is passed through the nanopore because the flanking strand is hybridised to the polynucleotide carrier strand. Accordingly, the polypeptide is passed through the nanopore. As the polypeptide is passed through the nanopore it is characterised.

The methods of the present invention can also be understood by reference to FIG. 6, which illustrates another non-limiting example of the disclosed methods.

A polynucleotide-polypeptide conjugate strand which comprises a target polypeptide flanked by one or more polynucleotide flanking strands may threaded through a nanopore.

In some embodiments the polynucleotide-polypeptide conjugate strand moves with respect to the pore. In some embodiments the target polypeptide is spanned by the polynucleotide flanking strand. Accordingly when the target polypeptide translocates through the nanopore it is spanned by a polynucleotide flanking strand and so the polynucleotide flanking strand and the target polypeptide co-translocate through the pore. In some embodiments the target polypeptide is spanned by the polynucleotide flanking strand and is attached to the polynucleotide flanking strand at one end of the target polypeptide. In some embodiments the target polypeptide is spanned by the polynucleotide flanking strand and is attached to the polynucleotide flanking strand at each end of the target polypeptide. In some embodiments each end of the target polypeptide is conjugated to a polynucleotide flanking strand and one or both polynucleotide flanking strands further span(s) the target polypeptide. In some embodiments one end of the target polypeptide is conjugated to a first polynucleotide flanking strand; the other end of the target polypeptide is conjugated to a second polynucleotide flanking strand; and one or both of the first and second polynucleotide flanking strands spans the target polypeptide. In some embodiments the portion of the flanking strand which spans the target polypeptide is the same type of polynucleotide as the remainder of the flanking strand. In some embodiments the portion of the flanking strand which spans the target polypeptide is a different type of polynucleotide to the remainder of the flanking strand.

In some embodiments the polynucleotide-polypeptide conjugate strand is hybridised to a polynucleotide carrier strand as described herein. In some embodiments the resulting construct translocates through the nanopore.

In some embodiments the movement of the construct through the nanopore leads to the co-translocation of three strands: the target polypeptide, the polynucleotide flanking strand that spans the target polypeptide and the polynucleotide carrier strand. This is shown in FIG. 6B. However, in some embodiments the polynucleotide carrier strand is removed (e.g. dehybridised thereby being unzipped) e.g. by the nanopore. In such embodiments, when the target polypeptide translocates through the nanopore it is spanned by the polynucleotide flanking strand and so the polynucleotide flanking strand and the target polypeptide co-translocate through the pore, and the polynucleotide carrier strand is excluded from the nanopore. This is shown in FIG. 6C.

In some embodiments the polynucleotide-polypeptide conjugate strand and/or the polynucleotide carrier strand is contacted with a polynucleotide-handling protein such that the construct threads the nanopore. However, this is not essential. Optionally, a further polymer such as a leader (described herein) may be used to facilitate the threading of the polypeptide through the nanopore. Such use is within the scope of the disclosed methods, however this is not essential. Optionally, a spacer and/or stall may be used to restrain the movement of the polynucleotide-handling protein (if present) prior to the operation of the methods by the user. This is described herein and is within the scope of the disclosed methods, but is not essential.

In the examples illustrated in FIGS. 1 and 6 the polynucleotide-handling protein moves the construct “into” the pore, from the “viewpoint” of the polynucleotide-handling protein. For example, as shown the polynucleotide-handling protein may be located on the cis side of the nanopore and move the construct into the pore, i.e. from the cis side to the trans side. The opposite setup can also be used.

In other words, in some embodiments, a polynucleotide-handling protein may be located on the cis side of the nanopore and the polynucleotide-handling protein controls the movement of the construct, polynucleotide-polypeptide conjugate strand and/or polynucleotide carrier strand from the cis side of the nanopore to the trans side of the nanopore. In some embodiments, the polynucleotide-handling protein is located on the cis side of the nanopore and the polynucleotide-handling protein controls the movement of the polynucleotide carrier strand from the cis side of the nanopore to the trans side of the nanopore, thereby controlling the movement of the target polypeptide through the nanopore. Thus, in some embodiments, the polynucleotide-handling protein is located on the cis side of the nanopore and the polynucleotide-handling protein controls the movement of the polypeptide from the cis side of the nanopore to the trans side of the nanopore, thereby controlling the movement of the polypeptide through the nanopore.

In other embodiments, the polynucleotide-handling protein is located on the trans side of the nanopore and the polynucleotide-handling protein controls the movement of the construct, polynucleotide-polypeptide conjugate strand and/or polynucleotide carrier strand from the trans side of the nanopore to the cis side of the nanopore. In some embodiments, the polynucleotide-handling protein is located on the trans side of the nanopore and the polynucleotide-handling protein controls the movement of the polynucleotide carrier strand from the trans side of the nanopore to the cis side of the nanopore, thereby controlling the movement of the target polypeptide through the nanopore. Thus, in some embodiments, the polynucleotide-handling protein is located on the trans side of the nanopore and the polynucleotide-handling protein controls the movement of the polypeptide from the trans side of the nanopore to the cis side of the nanopore, thereby controlling the movement of the polypeptide through the nanopore.

As explained herein in more detail, the construct may comprise a leader. A leader may be present on the polynucleotide-polypeptide conjugate strand. A leader may be present on the carrier strand if present.

A leader may be present on the same strand that is processed by a polynucleotide-handling protein if present. For example, in some embodiments the polynucleotide-polypeptide conjugate strand comprises a leader and a polynucleotide-handling protein controls the movement of the polynucleotide-polypeptide conjugate strand with respect to the nanopore. In other embodiments a carrier strand comprises a leader and a polynucleotide-handling protein controls the movement of the carrier strand with respect to the nanopore.

A leader may be present on one strand of the construct and a polynucleotide-handling protein may control the movement of the other strand of the construct. For example, in some embodiments the polynucleotide-polypeptide conjugate strand comprises a leader and a polynucleotide-handling protein controls the movement of the carrier strand with respect to the nanopore. In other embodiments a carrier strand comprises a leader and a polynucleotide-handling protein controls the movement of the polynucleotide-polypeptide conjugate strand with respect to the nanopore.

When a carrier strand is present, a leader may be attached to both the polynucleotide-polypeptide conjugate strand and the carrier strand. For example, a Y adapter as described herein may be used to attach the polynucleotide-polypeptide conjugate strand and the carrier strand. The stem of the Y adapter may provide a leader sequence, such as a single-stranded or double-stranded polynucleotide portion.

When present, a leader may comprise nucleotide units, spacer units, and/or other monomer units that can be attached together to form the leader. For example, a leader may comprise one or nucleotide units and/or one or more spacer units. Suitable spacer units are described in more detail here, and include e.g. one or more C3, iSp9 and/or iSp18 spacers as described herein.

Any suitable leader may be used, as explained herein. Optionally, the leader may be a polynucleotide. The leader may be the same as the polynucleotide in the conjugate or may be different. As explained above, the leader may facilitate the threading of the conjugate through the nanopore.

In other words, in some embodiments the polynucleotide-polypeptide conjugate strand comprises one or more structures of the form L-{P—N}—Pₘ, wherein:
L is a leader, wherein L is optionally an N moiety;
P is a polypeptide;
N comprises a polynucleotide; and
m is 0 or 1;
and the method may comprise threading the leader (L) through the nanopore thereby contacting the polypeptide (P) with the nanopore.

The polynucleotide-polypeptide conjugate strand may be hybridised to a carrier strand. The N portion(s) of the polynucleotide-polypeptide conjugate strand may be hybridised to one or more polynucleotide carrier strands. In some embodiments each N portion of the polynucleotide-polypeptide conjugate strand is hybridised to a polynucleotide carrier strand.

In some such embodiments, a polynucleotide-handling protein is located on the cis side of the nanopore and the method comprises allowing the polynucleotide-handling protein to control the movement of the polynucleotide moiety (N) from the cis side of the nanopore to the trans side of the nanopore, thereby controlling the movement of the polypeptide (P) through the nanopore. In other embodiments, the polynucleotide-handling protein is located on the trans side of the nanopore and the method comprises allowing the polynucleotide-handling protein to control the movement of the polynucleotide moiety (N) from the trans side of the nanopore to the cis side of the nanopore, thereby controlling the movement of the polypeptide (P) through the nanopore.

As explained in more detail herein, in some embodiments the construct comprises multiple polynucleotides and polypeptides. In such embodiments the polynucleotide-handling protein sequentially controls the movement of the polynucleotide-polypeptide conjugate strand and/or carrier strand with respect to the nanopore, thus sequentially moving each polypeptide with respect to the nanopore. In this way, each polypeptide within the conjugate can be sequentially characterised in the disclosed methods.

In some embodiments the polynucleotide-polypeptide conjugate strand comprises a plurality of target polypeptides. In some embodiments the polynucleotide-polypeptide conjugate strand comprises a plurality of target polypeptides. For example, the polynucleotide-polypeptide conjugate strand may comprise one or more moieties of form . . . N—P—N—P—N . . . wherein each N, which may be the same or different, is a polypeptide and wherein each N, which may be the same or different, comprises a polynucleotide. Some or all N portions of the polynucleotide-polypeptide conjugate strand may be hybridised to one or more polynucleotide carrier strands. In some embodiments each N portion of the polynucleotide-polypeptide conjugate strand is hybridised to a polynucleotide carrier strand.

For example, the polynucleotide-polypeptide conjugate strand may comprise one or more structures of the form L-P₁—N—{P—N}ₙ—Pₘ, wherein:
n is a positive integer;
L is a leader, wherein L is optionally an N moiety;
each P, which may be the same or different, is a polypeptide;
each N, which may be the same or different, comprises a polynucleotide; and
m is 0 or 1;
   and the method may comprise threading the leader (L) through the nanopore thereby contacting polypeptide (P₁) with the nanopore.

Typically, in such embodiments, n is from 1 to about 1000, e.g. from 2 to about 100,such as from about 3 to about 10, e.g. 1, 2, 3, 4, 5, 6, 7, 8, 9 or 10.

In some such embodiments, a polynucleotide-handling protein is located on the cis side of the nanopore and the method comprises allowing the polynucleotide-handling protein to control the movement of each polynucleotide (N) sequentially from the cis side of the nanopore to the trans side of the nanopore, thereby controlling the movement of each polypeptide (P) sequentially through the nanopore. In other such embodiments, the polynucleotide-handling protein is located on the trans side of the nanopore and the method comprises allowing the polynucleotide-handling protein to control the movement of each polynucleotide (N) sequentially from the trans side of the nanopore to the cis side of the nanopore, thereby controlling the movement of each polypeptide (P) sequentially through the nanopore.

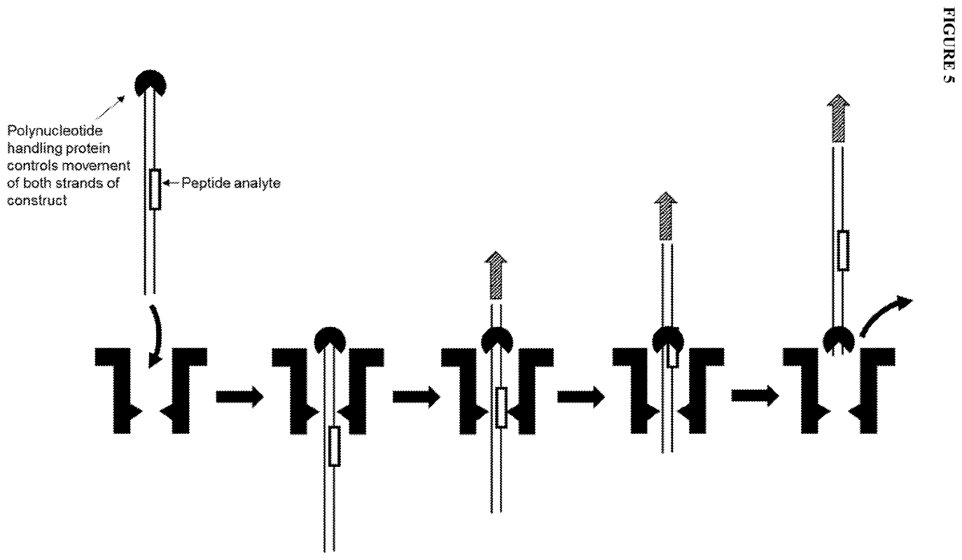

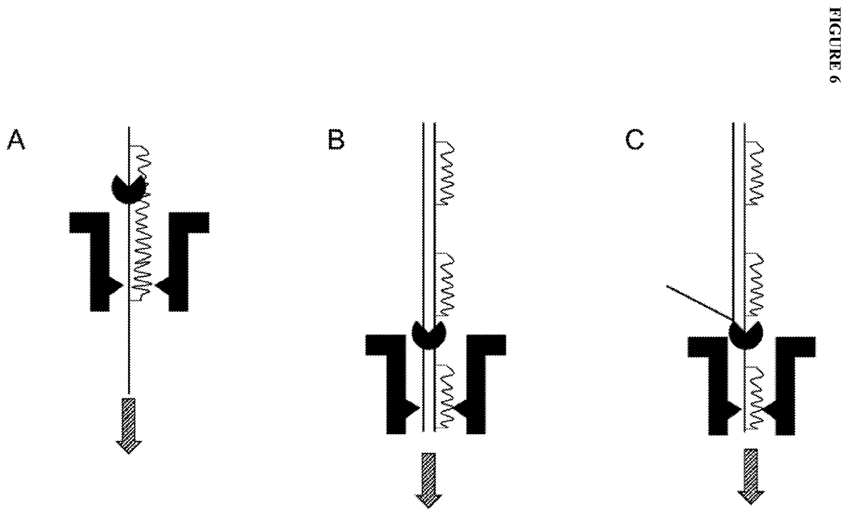

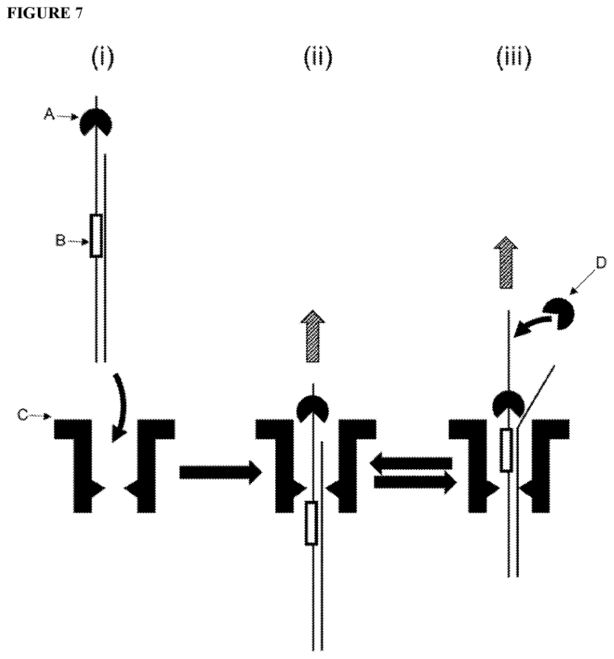

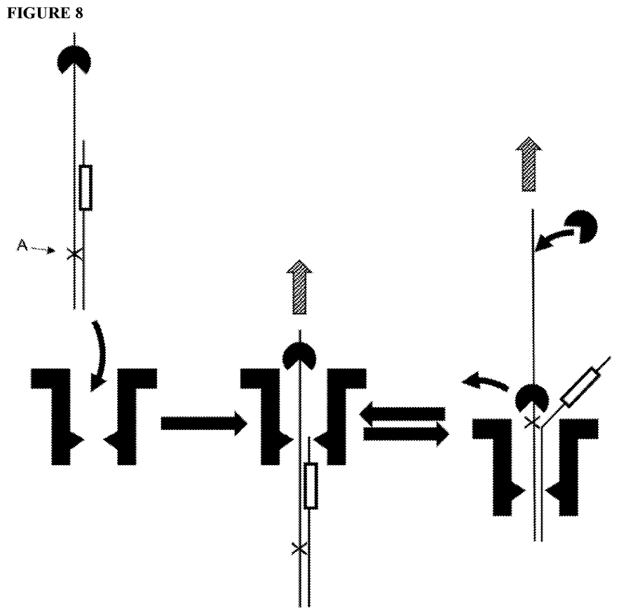

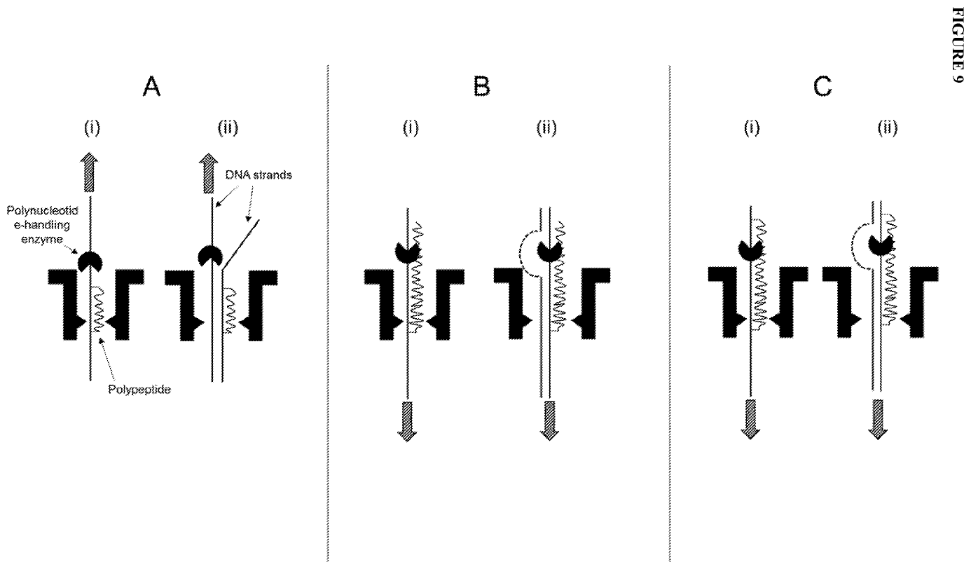

Those skilled in the art will appreciate that when the conjugate comprises more than one polypeptide, it may be advantageous that (as described in more detail herein) the polynucleotide-handling protein can remain bound to the conjugate when it contacts the polypeptide without dissociating. For example, as shown in FIGS. 4 and 5, this allows polynucleotide-handling protein to pass over portions of polypeptide in the conjugate as it contacts them, in order to move onto sequential portions of polynucleotide in order to control the movement of the conjugate with respect to the nanopore.

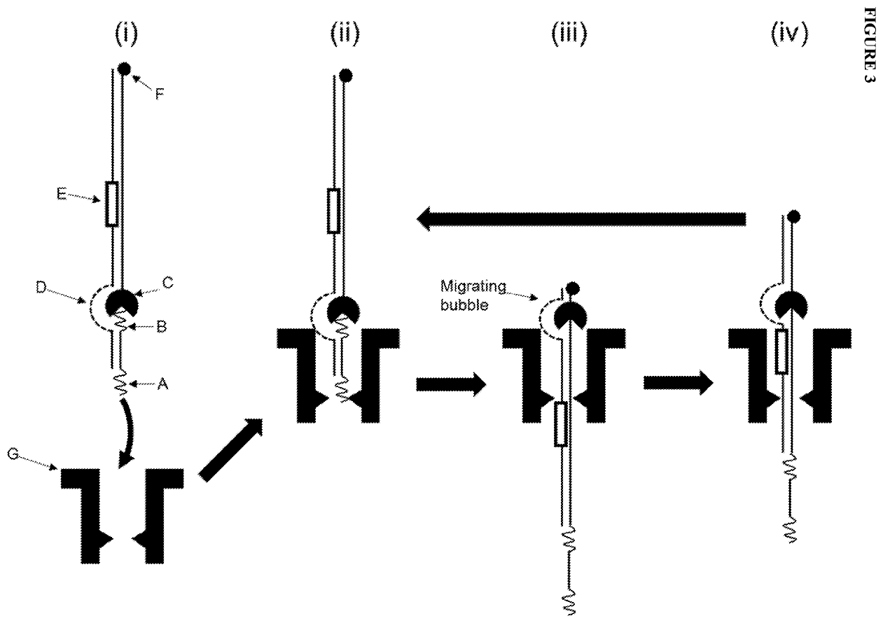

Another non-limiting embodiment of the disclosed methods is shown schematically in FIG. 2. In this example, a polynucleotide-handling protein moves the conjugate “out” of the pore, from the “viewpoint” of the polynucleotide-handling protein. For example, as shown the polynucleotide-handling protein is located on the cis side of the nanopore and moves the conjugate into the pore, i.e. from the trans side to the cis side. The opposite setup could also be used.

In other words, in some embodiments, a polynucleotide-handling protein may be located on the cis side of the nanopore and the polynucleotide-handling protein controls the movement of the construct, polynucleotide-polypeptide conjugate strand and/or polynucleotide carrier strand from the trans side of the nanopore to the cis side of the nanopore. In some embodiments, the polynucleotide-handling protein is located on the cis side of the nanopore and the polynucleotide-handling protein controls the movement of the polynucleotide carrier strand from the trans side of the nanopore to the cis side of the nanopore, thereby controlling the movement of the target polypeptide through the nanopore. Thus, in some embodiments the polynucleotide-handling protein is located on the cis side of the nanopore and the polynucleotide-handling protein controls the movement of the polypeptide from the trans side of the nanopore to the cis side of the nanopore, thereby controlling the movement of the polypeptide through the nanopore.

In other embodiments, the polynucleotide-handling protein is located on the trans side of the nanopore and the polynucleotide-handling protein controls the movement of the construct, polynucleotide-polypeptide conjugate strand and/or polynucleotide carrier strand from the cis side of the nanopore to the trans side of the nanopore. In some embodiments, the polynucleotide-handling protein is located on the trans side of the nanopore and the polynucleotide-handling protein controls the movement of the polynucleotide carrier strand from the cis side of the nanopore to the trans side of the nanopore, thereby controlling the movement of the target polypeptide through the nanopore. Thus, in some embodiments the polynucleotide-handling protein is located on the trans side of the nanopore and the polynucleotide-handling protein controls the movement of the polypeptide from the cis side of the nanopore to the trans side of the nanopore, thereby controlling the movement of the polypeptide through the nanopore.

Using similar notation as above, in some embodiments the polynucleotide-polypeptide conjugate strand may comprise one or more moieties of form . . . N—P—N—P—N . . . wherein each N, which may be the same or different, is a polypeptide and wherein each N, which may be the same or different, comprises a polynucleotide. Some or all N portions of the polynucleotide-polypeptide conjugate strand may be hybridised to one or more polynucleotide carrier strands. In some embodiments each N portion of the polynucleotide-polypeptide conjugate strand is hybridised to a polynucleotide carrier strand.

In some embodiments, the polynucleotide-polypeptide conjugate strand comprises one or more structures of the form L-{P—N}— Pₘ or L-P₁—N—{P—N}ₙ—Pₘ, wherein:
n is a positive integer;
L is a leader, wherein L is optionally an N moiety;
each P, which may be the same or different is a polypeptide;
each N, which may be the same or different comprises a polynucleotide;
m is 0 or 1;
   and the method may comprise threading the leader (L) through the nanopore thereby contacting the polypeptide (P) with the nanopore.

The polynucleotide-polypeptide conjugate strand may be hybridised to a carrier strand. The N portion(s) of the polynucleotide-polypeptide conjugate strand may be hybridised to one or more polynucleotide carrier strands. In some embodiments each N portion of the polynucleotide-polypeptide conjugate strand is hybridised to a polynucleotide carrier strand.

In some such embodiments the polynucleotide-handling protein is located on the cis side of the nanopore and the method comprises allowing the polynucleotide-handling protein to control the movement of the polynucleotide moieties (N) from the trans side of the nanopore to the cis side of the nanopore, thereby controlling the movement of the polypeptide moieties (P) through the nanopore. In other such embodiments the polynucleotide-handling protein is located on the trans side of the nanopore and the method comprises allowing the polynucleotide-handling protein to control the movement of the polynucleotide moieties (N) from the cis side of the nanopore to the trans side of the nanopore, thereby controlling the movement of the polypeptide moieties (P) through the nanopore

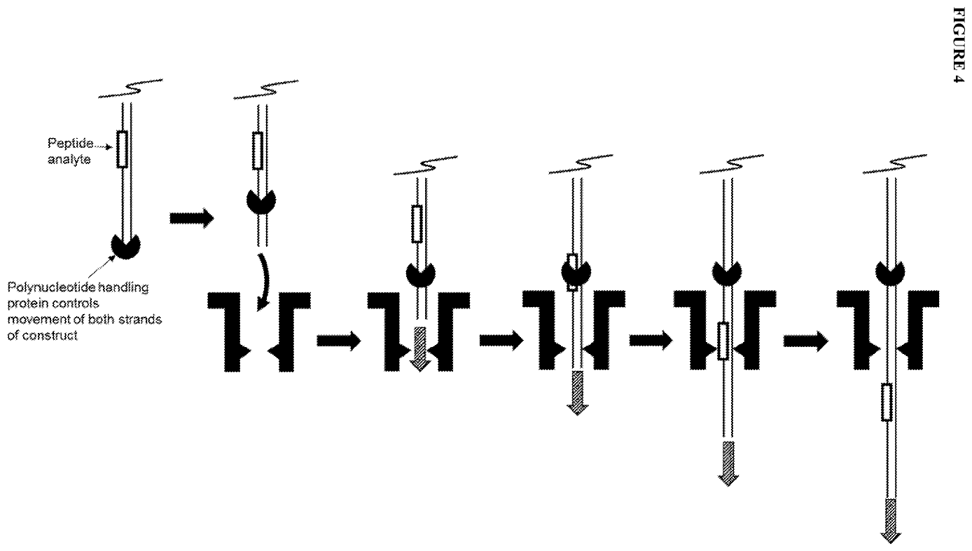

In some embodiments the methods provided herein comprise allowing the construct, polynucleotide-polypeptide conjugate strand and/or polynucleotide carrier strand to move back and forwards with respect to the nanopore. The process can be repeated multiple times. In such a manner, the polypeptide may oscillate through the pore (i.e. it may be “flossed” through the nanopore). This “flossing” allows the polypeptide portion of the conjugate to be repeatedly characterised by the nanopore. In some embodiments this allows the accuracy of the characterisation information to be increased. This “flossing” is also referred to herein as re-reading. One example of a re-reading method is shown in FIG. 3.

In some embodiments the methods comprise:
i) carrying out a method described herein such that the target polypeptide translocates the nanopore in a first direction with respect to the nanopore;
ii) allowing the construct, polynucleotide-polypeptide conjugate strand and/or polynucleotide carrier strand to move in a direction opposite to the direction of movement with respect to the nanopore in step (i) such that the target polypeptide translocates the nanopore in a second direction which is opposite to the first direction;
iii) optionally allowing the construct, polynucleotide-polypeptide conjugate strand and/or polynucleotide carrier strand to move in the first direction such that the target polypeptide re-translocates the nanopore in the first direction;
iv) optionally repeating steps (ii) and (iii) to oscillate the polypeptide through the nanopore.

In some embodiments, particularly in some embodiments which involve re-reading as described above, the conjugate may comprise a blocking moiety attached to the construct (e.g. to the polynucleotide-polypeptide conjugate strand and/or the carrier strand) via an optional linker.

The blocking moiety is typically too large to pass through the nanopore and so when the movement of the construct with respect to the nanopore brings the blocking moiety into contact with the nanopore, the further movement of the conjugate through the nanopore is prevented. In embodiments of the disclosed methods in which the conjugate moves with respect to the nanopore under an applied force (e.g. a voltage potential or chemical potential) the conjugate may then move “back” through the pore in the opposite direction to the movement controlled by the polynucleotide-handling protein. The movement of the conjugate back through the pore allows the polypeptide portion of the conjugate to be re-characterised in accordance with the disclosed methods again. This is illustrated schematically in FIG. 3 in which the circle at the top of the strand (here depicted by way of non-limiting example at the top of the carrier strand) represents the blocking moiety.

Any suitable blocking moiety can be used in such embodiments. For example, the conjugate may be modified with biotin and the blocking moiety may be e.g. streptavidin, avidin or neutravidin. The blocking moiety may be a large chemical group such as a dendrimer. The blocking moiety may be a nanoparticle or a bead. Other suitable blocking moieties will be apparent to those skilled in the art.

Accordingly, in some embodiments of the disclosed methods, the construct, polynucleotide-polypeptide conjugate strand and/or polynucleotide carrier strand comprises a blocking moiety attached via an optional linker, wherein the blocking moiety is incapable of translocating through the nanopore.

In some embodiments, the disclosed methods comprise
i) contacting the polynucleotide-polypeptide conjugate strand or a construct comprising the polynucleotide-polypeptide conjugate strand with the nanopore such that the blocking moiety is on the same side of the nanopore as a polynucleotide-handling protein;
ii) contacting a polynucleotide region of the construct with the polynucleotide-handling protein;
iii) allowing the polynucleotide-handling protein to control the movement of the polynucleotide with respect to the nanopore e.g. in a direction opposite to a force applied across the nanopore, e.g. a voltage potential; thereby controlling the movement of the polypeptide through the nanopore;
iv) allowing the construct to move with respect to e.g. in a direction with a force applied across the nanopore, e.g. a voltage potential; i.e. in a direction opposite to the direction of movement controlled by the polynucleotide-handling protein;
v) when the blocking moiety contacts the nanopore thereby preventing further movement of the conjugate through the nanopore, allowing the polynucleotide-handling protein to control the movement of the construct through the nanopore; and
vi) optionally repeating steps (iv) to (iv) to oscillate the polypeptide through the nanopore.

### Polypeptide

Any suitable polypeptide can be characterised in the disclosed methods.

As explained herein, in some embodiments multiple polypeptides are characterised. In some embodiments the polynucleotide-polypeptide conjugate strand comprises at least one, at least 2, at least 3, at least 4, at least 5, at least 6, at least 7, at least 8, at least 9, at least 10, at least 20, at least 30, at least 40, at least 50, or at least 100 polypeptide portions.

In some embodiments the or each target polypeptide is an unmodified protein or a portion thereof, or a naturally occurring polypeptide or a portion thereof.

In some embodiments the or each target polypeptide is secreted from cells. Alternatively, the or each target polypeptide can be produced inside cells such that it must be extracted from cells for characterisation by the disclosed methods. The or each polypeptide may comprise the products of cellular expression of a plasmid, e.g. a plasmid used in cloning of proteins in accordance with the methods described in Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley &Sons, New York (2016).

The or each polypeptide may be obtained from or extracted from any organism or microorganism. The or each polypeptide may be obtained from a human or animal, e.g. from urine, lymph, saliva, mucus, seminal fluid or amniotic fluid, or from whole blood, plasma or serum. The or each polypeptide may be obtained from a plant e.g. a cereal, legume, fruit or vegetable.

The or each target polypeptide can be provided as an impure mixture of one or more polypeptides and one or more impurities. Impurities may comprise truncated forms of the target polypeptide which are distinct from the “target polypeptides” for characterisation in the disclosed methods. For example, the or each target polypeptide may be a full length protein and impurities may comprise fractions of the protein. Impurities may also comprise proteins other than the target protein e.g. which may be co-purified from a cell culture or obtained from a sample.

A polypeptide may comprise any combination of any amino acids, amino acid analogs and modified amino acids (i.e. amino acid derivatives). Amino acids (and derivatives, analogs etc) in the polypeptide can be distinguished by their physical size and charge.

The amino acids/derivatives/analogs can be naturally occurring or artificial. In some embodiments the polypeptide does not comprise or consist of peptide nucleic acid (PNA).

In some embodiments the polypeptide may comprise any naturally occurring amino acid. Twenty amino acids are encoded by the universal genetic code. These are alanine (A), arginine (R), asparagine (N), aspartic acid (D), cysteine (C), glutamic acid/glutamate (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Other naturally occurring amino acids include selenocysteine and pyrrolysine.

In some embodiments the or each polypeptide is not modified, e.g. is not chemically modified as described in more detail herein. In some embodiments the or each polypeptide is not modified (e.g. chemically modified) for detection. In some embodiments the or each polypeptide does not comprise modified (e.g. chemically modified) amino acids. In some embodiments the polypeptide comprises unmodified amino acids. In some embodiments the polypeptide is an unmodified polypeptide. The polypeptide may thus for example be a naturally occurring polypeptide, or a synthetic polypeptide synthesized using unmodified (e.g. canonical) amino acids such as those set out in Table 1 above.

In some embodiments the or each polypeptide is modified. In some embodiments the or each polypeptide is modified for detection using the disclosed methods. In some embodiments the disclosed methods are for characterising modifications in the target polypeptide(s).

In some embodiments one or more of the amino acids/derivatives/analogs in the or each polypeptide is modified. In some embodiments one or more of the amino acids/derivatives/analogs in the or each polypeptide is post-translationally modified. As such, the methods disclosed herein can be used to detect the presence, absence, number of positions of post-translational modifications in a polypeptide. The disclosed methods can be used to characterise the extent to which a polypeptide has been post-translationally modified.

Any one or more post-translational modifications may be present in the or each polypeptide. Typical post-translational modifications include modification with a hydrophobic group, modification with a cofactor, addition of a chemical group, glycation (the non-enzymatic attachment of a sugar), biotinylation and pegylation. Post-translational modifications can also be non-natural, such that they are chemical modifications done in the laboratory for biotechnological or biomedical purposes. This can allow monitoring the levels of the laboratory made peptide, polypeptide or protein in contrast to the natural counterparts.

Examples of post-translational modification with a hydrophobic group include myristoylation, attachment of myristate, a C₁₄ saturated acid; palmitoylation, attachment of palmitate, a C₁₆ saturated acid; isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond.

Examples of post-translational modification with a cofactor include lipoylation, attachment of a lipoate (C₈) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation.

Examples of post-translational modification by addition of a chemical group include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; and ubiquitinilation, the addition of ubiquitin subunits (N-linked).

It is within the scope of the methods provided herein that the or each polypeptide is labelled with a molecular label. A molecular label may be a modification to the polypeptide which promotes the detection of the polypeptide in the methods provided herein. For example the label may be a modification to the polypeptide which alters the signal obtained as conjugate is characterised. For example, the label may interfere with a flux of ions through the nanopore. In such a manner, the label may improve the sensitivity of the methods.

In some embodiments the or each polypeptide contains one or more cross-linked sections, e.g. C—C bridges. In some embodiments the polypeptides is not cross-linked prior to being characterised using the disclosed methods.

In some embodiments the or each polypeptide comprises sulphide-containing amino acids and thus has the potential to form disulphide bonds. Typically, in such embodiments, the polypeptide is reduced using a reagent such as DTT (Dithiothreitol) or TCEP (tris(2-carboxyethyl)phosphine) prior to being characterised using the disclosed methods.

In some embodiments the or each polypeptide is a full length protein or naturally occurring polypeptide. In some embodiments a protein or naturally occurring polypeptide is fragmented prior to conjugation to the polynucleotide. In some embodiments the protein or polypeptide is chemically or enzymatically fragmented. In some embodiments polypeptides or polypeptide fragments can be conjugated to form a longer target polypeptide.

The or each polypeptide can be a polypeptide of any suitable length. In some embodiments the or each polypeptide independently has a length of from about 5 to about 5000 peptide units. In some embodiments the polypeptide has a length of from about 5 to about 1000 peptide units, for example from about 5 to about 500 peptide units, e.g. from about 5 to about 250 peptide units, such as from about 5 to about 100 peptide units, e.g. from about 5 to about 50 peptide units, e.g. from about 5 to about 25 peptide units. In some embodiments the or each polypeptide independently has a length of from about 10 to about 5000 peptide units. In some embodiments the or each polypeptide has a length of from about to about 1000 peptide units, for example from about 10 to about 500 peptide units, e.g. from about 10 to about 250 peptide units, such as from about 10 to about 100 peptide units, e.g. from about 10 to about 50 peptide units, e.g. from about 10 to about 25 peptide units. In some embodiments the or each polypeptide independently has a length of from about 25 to about 1000 peptide units, for example from about 50 to about 500 peptide units, e.g. from about 100 to about 250 peptide units.

Any number of polypeptides can be characterised in the disclosed methods. For instance, the method may comprise characterising 2, 3, 4, 5, 6, 7, 8, 9, 10, 20, 30, 50, 100 or more polypeptides. If two or more polypeptides are used, they may be different polypeptides or two or more instances of the same polypeptide.

It will thus be apparent that the measurements taken in the disclosed methods are typically characteristic of one or more characteristics of the polypeptide selected from (i) the length of the polypeptide, (ii) the identity of the polypeptide, (iii) the sequence of the polypeptide, (iv) the secondary structure of the polypeptide and (v) whether or not the polypeptide is modified. In typical embodiments the measurements are characteristic of the sequence of the polypeptide or whether or not the polypeptide is modified, e.g. by one or more post-translational modifications. In some embodiments the measurements are characteristics of the sequence of the polypeptide.

In some embodiments the polypeptide is in a relaxed form. In some embodiments the polypeptide is held in a linearized form. Holding the polypeptide in a linearized form can facilitate the characterisation of the polypeptide on a residue-by-residue basis as “bunching up” of the polypeptide within the nanopore is prevented.

The polypeptide can be held in a linearized form using any suitable means.

In some embodiments the polypeptide is held in a linearized form because it is attached to a polynucleotide flanking strand at each end of the polypeptide and the flanking strands are hybridised to complementary or substantially complementary portions of one or more polynucleotide carrier strands. The sequence of the flanking strands and/or the carrier strands can be chosen or designed such that the distance between the regions of the carrier strand which hybridise to the flanking strands is similar to the length of the polypeptide in a linearized form. The length of a polypeptide in a linearized form can be determined from the number of amino acids in the polypeptide if known, for example a peptide unit in a polypeptide is commonly considered to have a length of about 0.35 nm (3.5 Å). Thus, it is within the capacity of one of skill in the art to design flanking strands and carrier strands for any desired target polypeptide based, for example, on the mass of the polypeptide, which can be readily determined by methods known to those skilled in the art such as denaturing (e.g. SDS−) or native PAGE, or by mass spectrometry; or based on the sequence of the polypeptide if known.

Other methods of maintaining the target polypeptide in a linearized form are known.

For example, if the polypeptide is charged the polypeptide can be held in a linearized form by applying a voltage.

If the polypeptide is not charged or is only weakly charged then the charge can be altered or controlled by adjusting the pH. For example, the polypeptide can be held in a linearized form by using high pH to increase the relative negative charge of the polypeptide. Increasing the negative charge of the polypeptide allows it to be held in a linearized form under e.g. a positive voltage. Alternatively, the polypeptide can be held in a linearized form by using low pH to increase the relative positive charge of the polypeptide. Increasing the positive charge of the polypeptide allows it to be held in a linearized form under e.g. a negative voltage. In the disclosed methods a polynucleotide-handling protein is often used to control the movement of a polynucleotide with respect to a nanopore. As a polynucleotide is typically negatively charged it is generally most suitable to increase the linearization of the polypeptide by increasing the pH thus making the polypeptide more negatively charged, in common with the polynucleotide. In this way, the polynucleotide-polypeptide conjugate strand retains an overall negative charge and thus can readily move e.g. under an applied voltage.

The polypeptide can be held in a linearized form by using suitable denaturing conditions. Suitable denaturing conditions include, for example, the presence of appropriate concentrations of denaturants such as guanidine HCl and/or urea. The concentration of such denaturants to use in the disclosed methods is dependent on the target polypeptide to be characterised in the methods and can be readily selected by those of skill in the art.

The polypeptide can be held in a linearized form by using suitable detergents. Suitable detergents for use in the disclosed methods include SDS (sodium dodecyl sulfate).

The polypeptide can be held in a linearized form by carrying out the disclosed methods at an elevated temperature. Increasing the temperature overcomes intra-strand bonding and allows the polypeptide to adopt a linearized form.

The polypeptide can be held in a linearized form by carrying out the disclosed methods under strong electro-osmotic forces. Such forces can be provided by using asymmetric salt conditions and/or providing suitable charge in the channel of the nanopore. The charge in the channel of a protein nanopore can be altered e.g. by mutagenesis. Altering the charge of a nanopore is well within the capacity of those skilled in the art. Altering the charge of a nanopore generates strong electro-osmotic forces from the unbalanced flow of cations and anions through the nanopore when a voltage potential is applied across the nanopore.

### Formation of the Conjugate Strand

In forming the polynucleotide-polypeptide conjugate strand, the or each target polypeptide can be conjugate to the or each polynucleotide flanking strand at any suitable position. For example, the or each polypeptide can be conjugated to polynucleotide flanking strand(s) at the N-terminus or the C-terminus of the polypeptide. The polypeptide can be conjugated to the polynucleotide via a side chain group of a residue (e.g. an amino acid residue) in the polypeptide.

In some embodiments a target polypeptide has a naturally occurring reactive functional group which can be used to facilitate conjugation to the polynucleotide flanking strand(s). For example, a cysteine residue can be used to form a disulphide bond to the polynucleotide flanking strand(s) or to a modified group thereon.

In some embodiments a target polypeptide is modified in order to facilitate its conjugation to the polynucleotide flanking strand(s). For example, in some embodiments a polypeptide is modified by attaching a moiety comprising a reactive functional group for attaching to the polynucleotide flanking strand(s). For example, in some embodiments a polypeptide can be extended at the N-terminus or the C-terminus by one or more residues (e.g. amino acid residues) comprising one or more reactive functional groups for reacting with a corresponding reactive functional group on the polynucleotide flanking strand(s). For example, in some embodiments a polypeptide can be extended at the N-terminus and/or the C-terminus by one or more cysteine residues. Such residues can be used for attachment to the polynucleotide portion of the polynucleotide-polypeptide conjugate strand, e.g. by maleimide chemistry (e.g. by reaction of cysteine with an azido-maleimide compound such as azido-[Pol]-maleimide wherein [Pol] is typically a short chain polymer such as PEG, e.g. PEG2, PEG3, or PEG4; followed by coupling to appropriately functionalised polynucleotide e.g. polynucleotide carrying a BCN group for reaction with the azide). For avoidance of doubt, when the polypeptide comprises an appropriate naturally occurring residue at the N- and/or C-terminus (e.g. a naturally occurring cysteine residue at the N- and/or C-terminus) then such residue(s) can be used for attachment to the polynucleotide.

In some embodiments a residue in a target polypeptide is modified to facilitate attachment of the target polypeptide to the polynucleotide flanking strand(s). In some embodiments a residue (e.g. an amino acid residue) in the polypeptide is chemically modified for attachment to the polynucleotide flanking strand(s). In some embodiments a residue (e.g. an amino acid residue) in the polypeptide is enzymatically modified for attachment to the polynucleotide flanking strand(s).

The conjugation chemistry between the polynucleotide flanking strand(s) and the polypeptide portions of the polynucleotide-polypeptide conjugate strand is not particularly limited. Any suitable combination of reactive functional groups can be used. Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxmethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, vinylsulfonamides, and the like.

Other suitable chemistry for conjugating a polypeptide to a polynucleotide includes click chemistry. Many suitable click chemistry reagents are known in the art. Suitable examples of click chemistry include, but are not limited to, the following:
(a) copper(I)-catalyzed azide-alkyne cycloadditions (azide alkyne Huisgen cycloadditions);
(b) strain-promoted azide-alkyne cycloadditions; including alkene and azide [3+2]cycloadditions; alkene and tetrazine inverse-demand Diels-Alder reactions; and alkene and tetrazole photoclick reactions;
(c) copper-free variant of the 1,3 dipolar cycloaddition reaction, where an azide reacts with an alkyne under strain, for example in a cyclooctane ring such as in bicycle[6.1.0]nonyne (BCN);
(d) the reaction of an oxygen nucleophile on one linker with an epoxide or aziridine reactive moiety on the other; and
(e) the Staudinger ligation, where the alkyne moiety can be replaced by an aryl phosphine, resulting in a specific reaction with the azide to give an amide bond.

Any reactive group(s) may be used to form the conjugate. Some suitable reactive groups include [1, 4-Bis[3-(2-pyridyldithio)propionamido]butane; 1,1 1-bis-maleimidotriethyleneglycol; 3,3′-dithiodipropionic acid di(N-hydroxysuccinimide ester); ethylene glycol-bis(succinic acid N-hydroxysuccinimide ester); 4,4′-diisothiocyanatostilbene-2,2′-disulfonic acid disodium salt; Bis[2-(4-azidosalicylamido)ethyl]disulphide; 3-(2-pyridyldithio)propionic acid N-hydroxysuccinimide ester; 4-maleimidobutyric acid N-hydroxysuccinimide ester; Iodoacetic acid N-hydroxysuccinimide ester; S-acetylthioglycolic acid N-hydroxysuccinimide ester; azide-PEG-maleimide; and alkyne-PEG-maleimide. The reactive group may be any of those disclosed in WO 2010/086602, particularly in Table 3 of that application.

In some embodiments the reactive functional group is comprised in the polynucleotide and the target functional group is comprised in the polypeptide prior to the conjugation step. In other embodiments the reactive functional group is comprised in the polypeptide and the target functional group is comprised in the polynucleotide prior to the conjugation step. In some embodiments the reactive functional group is attached directly to the polypeptide. In some embodiments the reactive functional group is attached to the polypeptide via a spacer. Any suitable spacer can be used. Suitable spacers include for example alkyl diamines such as ethyl diamine, etc.

As explained above, in some embodiments the conjugate comprises a plurality of polypeptide sections and/or a plurality of polynucleotide sections. For example the conjugate may comprise a structure of the form . . . —P—N—P—N—P—N . . . wherein P is a polypeptide and N isa polynucleotide. In such embodiments the plurality of polynucleotides and polypeptides may be conjugated together by the same or different chemistries.

As explained herein, the construct may comprise a leader. The leader may be present on the polynucleotide-polypeptide conjugate strand (e.g. on the flanking strand) or the carrier strand if present. Any suitable leader may be used, as explained herein. In some embodiments the leader is, or comprises, a polynucleotide. In embodiments wherein the leader is a polynucleotide the leader may be the same sort of polynucleotide as the polynucleotide used in the conjugate, or it may be a different type of polynucleotide. For example, the polynucleotide in the conjugate may be DNA and the leader may be RNA or vice versa.

In some embodiments the leader may be from about 10 to 150 nucleotides (e.g. DNA and/or RNA nucleotides) in length, such as from 20 to 120, e.g. 30 to 100, for example 40 to 80 such as 50 to 70 nucleotides in length, or from about 10 to about 60 nucleotides in length, e.g. from about 20 to about 50, such as from about 20 to about 40, e.g. about 30 nucleotides in length.

In some embodiments the leader is a charged polymer, e.g. a negatively charged polymer. In some embodiments the leader comprises a polymer such as PEG or a polysaccharide. In such embodiments the leader may be from 10 to 150 monomer units (e.g. ethylene glycol or saccharide units) in length, such as from 20 to 120, e.g. 30 to 100, for example 40 to 80 such as 50 to 70 monomer units (e.g. ethylene glycol or saccharide units) in length.

### Polynucleotide

As explained in more detail herein, the methods provided herein comprise conjugating one or more polypeptides to one or more polynucleotide flanking strands. The resulting polynucleotide-polypeptide conjugate strand may be hybridised or otherwise attached to a polynucleotide carrier strand.

In the disclosed methods, any suitable polynucleotide can be used as the flanking strand(s) and/or the carrier strand(s). It is within the ability of one skilled in the art to select appropriate polynucleotides for use as flanking strand(s) and carrier strand(s) according to the target polypeptides to be characterised in the disclosed methods. For example, in some embodiments the or each flanking strand is at least as long as the target polypeptide. In some embodiments the or each flanking strand is shorter than the target polypeptide. In some embodiments a flanking strand spans the target polypeptide.

In some embodiments the or each polynucleotide used is secreted from cells. Alternatively, a polynucleotide can be produced inside cells such that it must be extracted from cells for use in the disclosed methods.

A polynucleotide may be provided as an impure mixture of one or more polynucleotides and one or more impurities. Impurities may comprise truncated forms of polynucleotides which are distinct from the polynucleotide for use in the formation of the conjugate. For example a polynucleotide for use as a flanking strand or carrier strand may be genomic DNA and impurities may comprise fractions of genomic DNA, plasmids, etc. The desired polynucleotide may be a coding region of genomic DNA and undesired polynucleotides may comprise non-coding regions of DNA.

Examples of polynucleotides include DNA and RNA. The bases in DNA and RNA may be distinguished by their physical size.

A polynucleotide or nucleic acid suitable for use as a flanking strand or carrier strand may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas.

One or more nucleotides in the polynucleotide may be modified, for instance with a label or a tag, for which suitable examples are known by a skilled person. A suitable for use as a flanking strand or carrier strand polynucleotide may comprise one or more spacers. An adapter, for example a sequencing adapter, may be comprised in the polynucleotide. Adapters, tags and spacers are described in more detail herein.

Examples of modified bases are disclosed herein and can be incorporated into the polynucleotide by means known in the art, e.g. by polymerase incorporation of modified nucleotide triphosphates during strand copying (e.g. in PCR) or by polymerase fill-in methods. In some embodiments one or more bases can be modified by chemical means using reagents known in the art.

A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers.

In general, a polynucleotide may be double stranded or single stranded. In the disclosed methods, the flanking strand(s) are typically single stranded. The carrier strand(s) are typically single stranded. Accordingly, the construct formed by attachment (e.g. by hybridisation) between the flanking strand and the carrier strand is thus double stranded.

A single-stranded polynucleotide suitable for use as a flanking strand or carrier strand is typically single stranded DNA. Single stranded RNA may be used. In some embodiments a polynucleotide suitable for use as a flanking strand or carrier strand is a single-stranded DNA-RNA hybrid. DNA-RNA hybrids can be prepared by ligating single stranded DNA to RNA or vice versa. The polynucleotide is most typically single stranded deoxyribonucleic acid (DNA) or single stranded ribonucleic nucleic acid (RNA), usually DNA.

In some embodiments the construct comprising the polynucleotide-polypeptide conjugate strand and a carrier strand construct comprises double stranded DNA. In some embodiments it comprises double stranded RNA. In some embodiments it comprises a double-stranded DNA-RNA hybrid. Double-stranded DNA-RNA hybrids can be prepared from single-stranded RNA by reverse transcribing the cDNA complement.

A polynucleotide suitable for use as a flanking strand or carrier strand can be any length. For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length.

More typically, a polynucleotide suitable for use as a flanking strand or carrier strand has a length of from about 1 to about 10,000 nucleotides or nucleotide pairs, such as from about 1 to about 1000 nucleotides or nucleotide pairs (e.g. from about 10 to about 1000 nucleotides or nucleotide pairs), e.g. from about 5 to about 500 nucleotides or nucleotide pairs, such as from about 10 to about 100 nucleotides or nucleotide pairs, e.g. from about 20 to about 80 nucleotides or nucleotide pairs such as from about 30 to about 50 nucleotides or nucleotide pairs.

Typically a polynucleotide suitable for use as a carrier strand is longer than a polynucleotide used as a flanking strand. Accordingly, typically multiple flanking strands and polypeptide sections can be associated with a single polynucleotide carrier strand.

For example, in some embodiments:
the or each target polypeptide has a length of from about 5 to about 1000 peptide units, for example from about 5 to about 500 peptide units, e.g. from about 5 to about 250 peptide units, such as from about 5 to about 100 peptide units, e.g. from about 5 to about 50 peptide units;
each flanking strand has a length of from about 5 to about 1000 nucleotides, for example from about 5 to about 500 nucleotides, e.g. from about 5 to about 250 nucleotides, such as from about 5 to about 100 nucleotides, e.g. from about 5 to about 50 nucleotides; and
each carrier strand has a length of from about 50 to about 50,000 nucleotides, such as from about 100 to about 10,000 nucleotides, e.g. about 5000 nucleotides. Much longer carrier strands may also be used, such as from about 1000 nucleotides to about 10 Mbase, such as from about 10,000 to about 1000000 nucleotides, e.g. about 100000 nucleotides.

In some embodiments, prior to the contacting the construct with the nanopore a polynucleotide-handling protein is bound to the polynucleotide carrier strand in a region of the polynucleotide carrier strand that is spanned by a non-hybridised region of the polynucleotide flanking strand. The flanking strand may thus form a bubble region around the polynucleotide-handling protein. The bubble region is a portion of the construct which is not hybridised because the polynucleotide-handling protein blocks the association of the flanking strand and the carrier strand. This is shown by way of non-limiting examples in schematic form in FIGS. 1 and 2.

The bubble region that spans the polynucleotide-handling protein when present may have any suitable length. The length is typically a function of the size of the polynucleotide-handling protein that is used. The bubble region that spans the polynucleotide-handling protein when present may have a length of for example from about 2 to about 50 nucleotides, such as from about 5 to about 20 nucleotides, e.g. about 19 nucleotides. In some embodiments the bubble region has a length of for example from about 2 to about 100 nucleotides, such as from about 10 to about 50 nucleotides, such as from about 20 to about 50 nucleotides, such as from about 30 to about 40, e.g. about 35 nucleotides.

In some embodiments the length of the portion of the corresponding strand around which the bubble may be formed (e.g., when the bubble is a portion of the flanking strand, the corresponding strand may be the carrier strand) may be, prior to the methods disclosed herein, from about 5 to about 20, such as from about 8 to about 12 e.g. about 10 nucleotide units. Thus, the bubble region is typically longer than the portion of the corresponding strand around which it is formed prior to the methods disclosed herein. For example, in some embodiments the bubble region has a length of about 10 to about 50 nucleotides, such as from about 30 to about 40 nucleotides, and the length of the portion of the corresponding strand about which the bubble is formed has a length of from about 5 to about 20, such as from about 8 to about 12 e.g. about 10 nucleotide units.

The bubble region that spans the polynucleotide-handling protein (prior to commencing the methods disclosed herein) may comprise or consist of any suitable type of polynucleotide. In some embodiments the portion of the polynucleotide flanking strand that spans the polynucleotide-handling protein is the same type of polynucleotide as the rest of the flanking strand. In some embodiments the portion of the polynucleotide flanking strand that spans the polynucleotide-handling protein is a different same type of polynucleotide to the rest of the flanking strand. In some embodiments the flanking strand comprises DNA polynucleotides and the bubble region (prior to commencing the disclosed methods) comprises or consists of RNA. In some embodiments the flanking strand comprises RNA and the bubble region (prior to commencing the disclosed methods) comprises or consists of DNA. In some embodiments the use of a different type of polynucleotide to form the initial bubble (as opposed to the remainder of the flanking strand) can serve to preferentially locate the polynucleotide-handling protein prior to commencement of the methods provided herein. In some embodiments the construct comprises a bubble region on one strand (e.g. on the flanking strand or the carrier strand) and the polynucleotide-handling protein is bound to the other strand of the construct (e.g. the carrier strand or the flanking strand). In some embodiments the polynucleotide-handling protein is stalled at a stalling moiety such as a spacer as described in more detail herein. In some embodiments the stalling moiety is opposite the bubble region. For example, in some embodiments the carrier strand comprises a spacer moiety for stalling the polynucleotide-handling protein prior to the commencement of the methods provided herein, and the polynucleotide-handling protein is bound to the carrier strand and is flanked by a bubble region on the polynucleotide-polypeptide conjugate strand. In some embodiments the polynucleotide-polypeptide conjugate strand comprises a spacer moiety for stalling the polynucleotide-handling protein prior to the commencement of the methods provided herein, and the polynucleotide-handling protein is bound to the polynucleotide-polypeptide conjugate strand and is flanked by a bubble region on the carrier strand.

As the polynucleotide-handling protein processes the construct the polynucleotide-handling protein moves along the strand(s) to which it is attached. The movement of the polynucleotide-handling protein along the strands thus results in the bubble region migrating along the strands with the polynucleotide-handling protein. This is shown schematically in FIGS. 1 and 2.

Any number of polynucleotides (e.g. any number of flanking strands) can be used in the disclosed methods. For instance, the method may comprise using 2, 3, 4, 5, 6, 7, 8, 9, 10, 20, 30, 50, 100 or more polynucleotides (e.g. polynucleotide flanking strands). If two or more polynucleotides are used, they may be different polynucleotides or two instances of the same polynucleotide. The polynucleotide can be naturally occurring or artificial. Typically, the polynucleotide-polypeptide conjugate strand comprises one flanking strand separating each target polypeptide; thus often the number of polynucleotide flanking strands is the same as or substantially the same as the number of polypeptides.

Nucleotides can have any identity, and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e. lack a nucleobase). A nucleotide may also lack a nucleobase and a sugar (i.e. is a C3 spacer).

A polynucleotide may comprise the products of a PCR reaction, genomic DNA, the products of an endonuclease digestion and/or a DNA library. A polynucleotide may be obtained from or extracted from any organism or microorganism. A polynucleotide may be obtained from a human or animal, e.g. from urine, lymph, saliva, mucus, seminal fluid or amniotic fluid, or from whole blood, plasma or serum. A polynucleotide may be obtained from a plant e.g. a cereal, legume, fruit or vegetable. A polynucleotide may comprise genomic DNA. The genomic DNA may be fragmented. A DNA may be fragmented by any suitable method. For example, methods of fragmenting DNA are known in the art, Such methods may use a transposase, such as a MuA transposase. Often the genomic DNA is not fragmented.

It is within the scope of the methods provided herein that the polynucleotide is labelled with a molecular label. A molecular label may be a modification to the polynucleotide which promotes the detection of the polynucleotide or conjugate in the methods provided herein. For example the label may be a modification to the polynucleotide which alters the signal obtained as conjugate is characterised. For example, the label may interfere with a flux of ions through the nanopore. In such a manner, the label may improve the sensitivity of the methods.

### Adapters

In some embodiments of the methods provided herein, a polynucleotide (e.g. a flanking strand or carrier strand) may have a polynucleotide adapter attached thereto. An adapter typically comprises a polynucleotide strand capable of being attached to the end of the polynucleotide.

In some embodiments the adapter is attached to a flanking strand before the conjugate with the polypeptide is formed. In some embodiments the adapter is attached to the conjugate of a polynucleotide and the polypeptide.

Accordingly, in some embodiments the methods comprise attaching an adapter (e.g. an adapter as described herein) to a polynucleotide and forming the polynucleotide-polypeptide conjugate strand by conjugating the polynucleotide/adapter construct to the target polypeptide. In some embodiments the polynucleotide-polypeptide conjugate strand is formed by attaching an adapter (e.g. an adapter as described herein) to the polynucleotide and forming the conjugate by attaching the adapter to the target polypeptide.

In some embodiments the adapter may be chosen or modified in order to provide a specific site for the conjugation to the polynucleotide.

An adapter may be attached to just one end of a polynucleotide or conjugate. A polynucleotide adapter may be added to both ends of a polynucleotide or conjugate. Alternatively, different adapters may be added to the two ends of a polynucleotide or conjugate.

Adapters may be added to both strands of double stranded polynucleotides (e.g. constructs as described herein). Adapter may be added to single stranded polynucleotides (e.g. polynucleotide-polypeptide conjugate strands as described herein). Methods of adding adapters to polynucleotides are known in the art. Adapters may be attached to polynucleotides, for example, by ligation, by click chemistry, by tagmentation, by topoisomerisation or by any other suitable method.

In one embodiment, the or each adapter is synthetic or artificial. Typically, the or each adapter comprises a polymer as described herein. In some embodiments, the or each adapter comprises a spacer as described herein. In some embodiments, the or each adapter comprises a polynucleotide. The or each polynucleotide adapter may comprise DNA, RNA, modified DNA (such as abasic DNA), RNA, PNA, LNA, BNA and/or PEG. Usually, the or each adapter comprises single stranded and/or double stranded DNA or RNA. The adapter may comprise the same type of polynucleotide as the polynucleotide strand to which it is attached. The adapter may comprise a different type of polynucleotide to the polynucleotide strand to which it is attached. In some embodiments the polynucleotide strand used in the disclosed methods is a single stranded DNA strand and the adapter comprises DNA or RNA, typically single stranded DNA. In some embodiments the polynucleotide is a double stranded DNA strand and the adapter comprises DNA or RNA, e.g. double or single stranded DNA.

In some embodiments, an adapter may be a bridging moiety. A bridging moiety may be used to connect the two strands of a double-stranded polynucleotide. For example, in some embodiments a bridging moiety is used to connect the template strand of a double stranded polynucleotide to the complement strand of the double stranded polynucleotide. For example, a bridging adapter may be used to connect a flanking strand to a carrier strand.

A bridging moiety typically covalently links the two strands of a double-stranded polynucleotide. The bridging moiety can be anything that is capable of linking the two strands of a double-stranded polynucleotide, provided that the bridging moiety does not interfere with movement of the polynucleotide with respect to the nanopore. Suitable bridging moieties include, but are not limited to a polymeric linker, a chemical linker, a polynucleotide or a polypeptide. Preferably, the bridging moiety comprises DNA, RNA, modified DNA (such as abasic DNA), RNA, PNA, LNA or PEG. The bridging moiety is more preferably DNA or RNA.

In some embodiments a bridging moiety is a hairpin adapter. A hairpin adapter is an adapter comprising a single polynucleotide strand, wherein the ends of the polynucleotide strand are capable of hybridising to each other, or are hybridized to each other, and wherein the middle section of the polynucleotide forms a loop. Suitable hairpin adapters can be designed using methods known in the art. In some embodiments a hairpin loop is typically 4 to 100 nucleotides in length, e.g. from 4 to 50 such as from 4 to 20 e.g. from 4 to 8 nucleotides in length. In some embodiments the bridging moiety (e.g. hairpin adapter) is attached at one end of a double-stranded polynucleotide. A bridging moiety (e.g. hairpin adapter) is typically not attached at both ends of a double-stranded polynucleotide.

In some embodiments, an adapter is a linear adapter. A linear adapter may be bound to either or both ends of a single stranded polynucleotide. When the polynucleotide is a double stranded polynucleotide, a linear adapter may be bound to either or both ends of either or both strands of the double stranded polynucleotide. A linear adapter may be attached to either or both ends of either or both of the polynucleotide-polypeptide conjugate strand and the carrier strand if present.

A linear adapter may comprise a leader sequence as described herein. A linear adapter may comprise a portion for hybridisation with a tag (such as a pore tag) as described herein. A linear adapter may be 10 to 150 nucleotides in length, such as from 20 to 120, e.g. to 100, for example 40 to 80 such as 50 to 70 nucleotides in length. A linear adapter may be single stranded. A linear adapter may be double stranded.

In some embodiments, an adapter may be a Y adapter. A Y adapter is typically a polynucleotide adapter. A Y adapter is typically double stranded and comprises (a) at one end, a region where the two strands are hybridised together and (b), at the other end, a region where the two strands are not complementary. The non-complementary parts of the strands typically form overhangs. The presence of a non-complementary region in the Y adapter gives the adapter its Y shape since the two strands typically do not hybridise to each other unlike the double stranded portion. The two single-stranded portions of the Y adapter may be the same length, or may be different lengths. For example, one single-stranded portion of the Y adapter may be 10 to 150 nucleotides in length, such as from 20 to 120, e.g. 30 to 100, for example 40 to 80 such as 50 to 70 nucleotides in length and the other single stranded portion of the Y adapter may independently by 10 to 150 nucleotides in length, such as from 20 to 120, e.g. 30 to 100, for example 40 to 80 such as 50 to 70 nucleotides in length. The double-stranded “stem” portion of the Y adapter may be e.g. from 10 to 150 nucleotides in length, such as from 20 to 120, e.g. 30 to 100, for example 40 to 80 such as 50 to 70 nucleotides in length. A Y adapter may be attached to either or both ends of the construct described herein.

An adapter may be linked to a polynucleotide (e.g. to the carrier strand or flanking strand) by any suitable means known in the art. The adapter may be synthesized separately and chemically attached or enzymatically ligated to the polynucleotide. Alternatively, the adapter may be generated in the processing of the polynucleotide. In some embodiments, the adapter is linked to the polynucleotide at or near one end of the target polynucleotide. In some embodiments, the adapter is linked to the polynucleotide within 50, e.g. within 20 for example within 10 nucleotides of an end of the polynucleotide. In some embodiments the adapter is linked to the polynucleotide at a terminus of the polynucleotide. When an adapter is linked to the polynucleotide the adapter may comprise the same type of nucleotides as the polynucleotide or may comprise different nucleotides to the polynucleotide.

Adapters which are particularly suitable for use in the disclosed methods may comprise linear homopolymeric regions (e.g. from about 5 to about 20 nucleotides, such as from about 10 to about 30 nucleotides, for example thymine or cytidine) and/or hybridisation sites for hybridising to one or more tethers or anchors (as described in more detail herein). Such adapters may also comprise reactive functional groups for binding to the target polypeptide. Click chemistry groups are particularly suitable in this regard. For example, exemplary groups for inclusion in an adapter include groups which can particulate in copper-free click chemistry, for example groups based on BCN (bicyclo[6.1.0]nonyne) and its derivatives, dibenzocyclooctyne (DBCO) groups, and the like. The reaction of such groups is well known in the art. For example, BCN groups typically react with groups such as azides, tetrazines and nitrones, which can for example incorporated in the polypeptide. DBCO groups have high reactivity toward azide groups. Other chemical groups which are particularly suitable include 2-pyridinecarboxyaldehyde (2-PCA) groups and their derivatives. For example, 6-(azidomethyl)-2-pyridinecarboxyaldehyde can react with N-terminal amino groups of peptides.

### Spacers

In some embodiments of the methods provided herein, a polynucleotide, a conjugate formed by the reaction thereof with a polypeptide, a leader, or an adapter as described herein, may comprise a spacer. For example, one or more spacers may be present in a polynucleotide adapter. One or more spacers may be present in an adapter attached to the flanking strand or carrier strand.

For example, a polynucleotide adapter may comprise from one to about 20 spacers, e.g. from about 1 to about 10, e.g. from 1 to about 5 spacers, e.g. 1, 2, 3, 4 or 5 spacers. The spacer may comprise any suitable number of spacer units. A spacer may provide an energy barrier which impedes movement of a polynucleotide-handling protein. For example, a spacer may stall a polynucleotide-handling protein by reducing the traction of the polynucleotide-handling protein on the polynucleotide. This may be achieved for instance by using an abasic spacer i.e. a spacer in which the bases are removed from one or more nucleotides in the polynucleotide adapter. A spacer may physically block movement of a polynucleotide-handling protein, for instance by introducing a bulky chemical group to physically impede the movement of the polynucleotide-handling protein.

In some embodiments, one or more spacers are included in a polynucleotide or conjugate or in an adapter as used in the methods claimed herein in order to provide a distinctive signal when they pass through or across the nanopore, i.e. as they move with respect to the nanopore.

In some embodiments, a spacer may comprise a linear molecule, such as a polymer. Typically, such a spacer has a different structure from the polynucleotide used in the conjugate. For instance, if the polynucleotide is DNA, the or each spacer typically does not comprise DNA. In particular, if the polynucleotide is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA) or a synthetic polymer with nucleotide side chains. In some embodiments, a spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9)[(OCH₂CH₂)₃OPO₃] groups, or one or more spacer 18 (iSp18) [(OCH₂CH₂)₆₀PO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units.

In some embodiments, a spacer may comprise one or more chemical groups which cause a polynucleotide-handling protein to stall. In some embodiments, suitable chemical groups are one or more pendant chemical groups. The one or more chemical groups may be attached to one or more nucleobases in the polynucleotide, construct or adapter. The one or more chemical groups may be attached to the backbone of the polynucleotide adapter. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups. In some embodiments, a spacer may comprise a polymer. In some embodiments the spacer may comprise a polymer which is a polypeptide or a polyethylene glycol (PEG).

In some embodiments, a spacer may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

Methods of stalling a polynucleotide-handling protein such as a helicase on a polynucleotide adapter using a spacer are described in WO 2014/135838, which is hereby incorporated by reference in its entirety.

In some embodiments the construct comprises a stalling moiety and prior to the translocation of the target polypeptide through the nanopore the polynucleotide-handling protein is positioned such that the stalling moiety is located between the polynucleotide-handling protein and the target polypeptide. In some embodiments this can be advantageous as it prevents the polynucleotide-handling protein from processing the portion of the construct corresponding to the target polypeptide before the start of the measurement.

In some embodiments the construct comprises a stalling moiety at the junction between the target polypeptide and one or more polynucleotide flanking strands. In some embodiments the one or more flanking strands are conjugated to the target polypeptide stand via click chemistry as described in more detail herein. In some embodiments the polynucleotide-handling protein stalls at the click chemistry junction.

### Anchors

In some embodiments, a polynucleotide, conjugate thereof with a polypeptide, or an adapter attached thereto may comprise a membrane anchor or a transmembrane pore anchor e.g. attached to the adapter. In one embodiment the anchor aids in characterisation of the conjugate in accordance with the methods disclosed herein. For example, a membrane anchor or transmembrane pore anchor may promote localisation of the conjugate around a nanopore in a membrane.

The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol. The anchor may comprise thiol, biotin or a surfactant.

In one embodiment the anchor may be biotin (for binding to streptavidin), amylose (for binding to maltose binding protein or a fusion protein), Ni-NTA (for binding to poly-histidine or poly-histidine tagged proteins) or peptides (such as an antigen).

In one embodiment, the anchor comprises a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The adapter may hybridise to a complementary sequence on a circular polynucleotide linker. The one or more anchors or one or more linkers may comprise a component that can be cut or broken down, such as a restriction site or a photolabile group. The linker may be functionalised with maleimide groups to attach to cysteine residues in proteins. Suitable linkers are described in WO 2010/086602.

In one embodiment, the anchor is cholesterol or a fatty acyl chain. For example, any fatty acyl chain having a length of from 6 to 30 carbon atom, such as hexadecanoic acid, may be used. Examples of suitable anchors and methods of attaching anchors to adapters are disclosed in WO 2012/164270 and WO 2015/150786.

Controlling Movement of the Conjugate with Respect to a Nanopore

As explained above, the methods provided herein comprise controlling the movement of a polynucleotide-polypeptide conjugate strand optionally bound (e.g. hybridised) to a carrier strand as the conjugate or construct moves with respect to a nanopore.

The movement of the conjugate with respect to the nanopore may be driven by any suitable means. In some embodiments, the movement of the conjugate is driven by a physical or chemical force (potential). In some embodiments the physical force is provided by an electrical (e.g. voltage) potential or a temperature gradient, etc.

In some embodiments, the movement of the construct comprises mechanically manipulating the construct, polynucleotide-polypeptide conjugate strand and/or polynucleotide carrier strand thereby moving said construct, polynucleotide-polypeptide conjugate strand and/or polynucleotide carrier strand with respect to the nanopore. In some embodiments, movement of the construct by mechanical manipulation does not comprise using a polynucleotide-handling protein.

In some embodiments the construct, polynucleotide-polypeptide conjugate strand and/or polynucleotide carrier strand is moved by mechanical manipulation in a direction opposite to a potential applied across said nanopore. In some embodiments, the potential is a voltage potential applied across said nanopore. In some embodiments, the construct, polynucleotide-polypeptide conjugate strand and/or polynucleotide carrier strand is moved with respect to the nanopore as described in WO 2020/128517, the entire contents of which are hereby incorporated by reference, particularly in regards to discussion in that document of movements of polynucleotides with respect to nanoreactors.

In some embodiments, the conjugate moves with respect to the nanopore as an electrical potential is applied across the nanopore. Polynucleotides are negatively charged, and so applying a voltage potential across a nanopore will cause the polynucleotides to move with respect to the nanopore under the influence of the applied voltage potential. For example, if a positive voltage potential is applied to the trans side of the nanopore relative to the cis side of the nanopore, then this will induce a negatively charged analyte to move from the cis side of the nanopore to the trans side of the nanopore. Similarly, if a positive voltage potential is applied to the trans side of the nanopore relative to the cis side of the nanopore then this will impede the movement of a negatively charged analyte from the trans side of the nanopore to the cis side of the nanopore. The opposite will occur if a negative voltage potential is applied to the trans side of the nanopore relative to the cis side of the nanopore. Apparatuses and methods of applying appropriate voltages are described in more detail herein.

In some embodiments the chemical force is provided by a concentration (e.g. pH) gradient.

In some embodiments the movement of the conjugate or construct with respect to the nanopore is controlled using a method as described in WO 2020/016573, the entire contents of which are incorporated herein by reference.

In some embodiments, a pausing group may be non-covalently attached to the construct before the method is undertaken. A pausing group, such as a short oligonucleotide capable of hybridising onto the construct, may transiently halting the movement of said construct with respect to the nanopore. This can be usefully employed to allow the polypeptide portion of the polynucleotide-polypeptide conjugate strand to be transiently held in the pore for an increased time to facilitate its characterisation.

In some embodiments the polynucleotide-handling protein controls the movement of the construct, polynucleotide-polypeptide conjugate strand and/or carrier strand in the same direction as the physical or chemical force (potential). For example, in some embodiments a positive voltage potential is applied to the trans side of the nanopore relative to the cis side of the nanopore, and the polynucleotide-handling protein controls the movement of the construct from the cis side of the nanopore to the trans side of the nanopore. In some embodiments a positive voltage potential is applied to the cis side of the nanopore relative to the trans side of the nanopore, and the polynucleotide-handling protein controls the movement of the construct from the trans side of the nanopore to the cis side of the nanopore.

In some embodiments the polynucleotide-handling protein controls the movement of the construct, polynucleotide-polypeptide conjugate strand and/or carrier strand in the opposite direction to the physical or chemical force (potential). For example, in some embodiments a positive voltage potential is applied to the trans side of the nanopore relative to the cis side of the nanopore, and the polynucleotide-handling protein controls the movement of the construct from the trans side of the nanopore to the cis side of the nanopore. In some embodiments a positive voltage potential is applied to the cis side of the nanopore relative to the trans side of the nanopore, and the polynucleotide-handling protein controls the movement of the construct from the cis side of the nanopore to the trans side of the nanopore.

In some embodiments the movement of the construct, polynucleotide-polypeptide conjugate strand and/or carrier strand is driven by the polynucleotide-handling protein in the absence of an applied potential.

In the disclosed methods, the polynucleotide-handling protein is typically capable of controlling the movement of the construct, polynucleotide-polypeptide conjugate strand and/or carrier strand with respect to a nanopore. In other words, the polynucleotide-handling protein is capable of controlling the movement of the construct.

In some embodiments, the disclosed methods comprise contacting the construct with a polynucleotide-handling protein capable of controlling the movement of the one or more polynucleotide flanking strands and/or the polynucleotide carrier strand, wherein the polynucleotide-handling protein controls the movement of the construct with respect to the nanopore. In some embodiments, the disclosed methods comprise contacting the construct with a polynucleotide-handling protein, and controlling the movement of the one or more polynucleotide flanking strands and/or the polynucleotide carrier strand using the polynucleotide-handling protein thereby controlling the movement of the construct with respect to the nanopore.

In some embodiments, the disclosed methods comprise contacting both polynucleotide-polypeptide conjugate strand and the polynucleotide carrier strand of the construct with a polynucleotide-handling protein capable of controlling the movement of the polynucleotide-polypeptide conjugate strand and/or the polynucleotide carrier strand, wherein the polynucleotide-handling protein controls the movement of the construct with respect to the nanopore. In some embodiments, the disclosed methods comprise contacting both polynucleotide-polypeptide conjugate strand and the polynucleotide carrier strand of the construct with a polynucleotide-handling protein, and controlling the movement of the polynucleotide-polypeptide conjugate strand and/or the polynucleotide carrier strand using the polynucleotide-handling protein thereby controlling the movement of the construct with respect to the nanopore.

In some embodiments, the disclosed methods comprise contacting the polynucleotide-polypeptide conjugate strand with a polynucleotide-handling protein capable of controlling the movement of the polynucleotide-polypeptide conjugate strand, wherein the polynucleotide-handling protein controls the movement of the construct with respect to the nanopore. In some embodiments, the disclosed methods comprise contacting the polynucleotide-polypeptide conjugate strand with a polynucleotide-handling protein, and controlling the movement of the polynucleotide-polypeptide conjugate strand using the polynucleotide-handling protein thereby controlling the movement of the construct with respect to the nanopore.

In some embodiments, the disclosed methods comprise contacting the polynucleotide carrier strand with a polynucleotide-handling protein capable of controlling the movement of the polynucleotide carrier strand, wherein the polynucleotide-handling protein controls the movement of the construct with respect to the nanopore. In some embodiments, the disclosed methods comprise contacting the polynucleotide carrier strand with a polynucleotide-handling protein, and controlling the movement of the polynucleotide carrier strand using the polynucleotide-handling protein thereby controlling the movement of the construct with respect to the nanopore.

As the construct moves with respect to the nanopore the target polypeptide of the construct moves with respect to the nanopore and can be thereby characterised.

Suitable polynucleotide-handling proteins are also known as motor proteins or polynucleotide-handling enzymes. Suitable polynucleotide-handling proteins are known in the art and some exemplary polynucleotide-handling proteins are described in more detail below.

In one embodiment, a motor protein is or is derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position.

In some embodiments, a polynucleotide-handling protein can be present on the construct prior to its contact with a nanopore. For example, a polynucleotide-handling protein can be present on a polynucleotide (e.g. the flanking strand or carrier strand) in the conjugate. In some embodiments the polynucleotide-handling protein is present on an adapter comprising part of the conjugate, or can be otherwise present on a portion of the conjugate.

In some embodiments the polynucleotide-handling protein is capable of remaining bound to the polynucleotide-polypeptide conjugate strand when the portion of the polynucleotide-polypeptide conjugate strand in contact with the active site of the polynucleotide-handling protein comprises a polypeptide. In other words, in some embodiments the polynucleotide-handling protein does not dissociate from the polynucleotide-polypeptide conjugate strand when the polynucleotide-handling protein contacts the polypeptide portion of the polynucleotide-polypeptide conjugate strand. In some embodiments the polynucleotide-handling protein moves freely with respect to the polypeptide portion until one or more subsequent polynucleotide portions of the polynucleotide-polypeptide conjugate strand are contacted.

In some embodiments the polynucleotide-handling protein is modified to prevent it from disengaging from the construct (e.g. from the polynucleotide-polypeptide conjugate strand or carrier strand) (other than by passing off the end of the construct or strand) when the polynucleotide-handling protein contacts a portion of the conjugate comprising a polypeptide. Such modified polynucleotide-handling proteins are particularly suitable for use in the disclosed methods.

The polynucleotide-handling protein can be adapted in any suitable way. For example, the polynucleotide-handling protein can be loaded onto the construct, polynucleotide-polypeptide conjugate strand and/or polynucleotide carrier strand and then modified in order to prevent it from disengaging. Alternatively, the polynucleotide-handling protein can be modified to prevent it from disengaging before it is loaded onto the construct, polynucleotide-polypeptide conjugate strand and/or polynucleotide carrier strand. Modification of a polynucleotide-handling protein in order to prevent it from disengaging from a construct, polynucleotide-polypeptide conjugate strand and/or polynucleotide carrier strand can be achieved using methods known in the art, such as those discussed in WO 2014/013260, which is hereby incorporated by reference in its entirety, and with particular reference to passages describing the modification of polynucleotide-handling proteins (polynucleotide binding proteins) such as helicases in order to prevent them from disengaging with polynucleotide strands.

For example, the polynucleotide-handling protein may have a polynucleotide-unbinding opening; e.g. a cavity, cleft or void through which a polynucleotide strand may pass when the polynucleotide-handling protein disengages from the strand. In some embodiments, the polynucleotide-unbinding opening for a given motor protein (polynucleotide-handling protein) can be determined by reference to its structure, e.g. by reference to its X-ray crystal structure. The X-ray crystal structure may be obtained in the presence and/or the absence of a polynucleotide substrate. In some embodiments, the location of a polynucleotide-unbinding opening in a given polynucleotide-handling protein may be deduced or confirmed by molecular modelling using standard packages known in the art. In some embodiments, the polynucleotide-unbinding opening may be transiently produced by movement of one or more parts e.g. one or more domains of the polynucleotide-handling protein.

The polynucleotide-handling protein (motor protein) may be modified by closing the polynucleotide-unbinding opening. Closing the polynucleotide-unbinding opening may therefore prevent the polynucleotide-handling protein from disengaging from the polypeptide portion of the conjugate as well as preventing it from disengaging from the polynucleotide or adapter. For example, the motor protein may be modified by covalently closing the polynucleotide-unbinding opening. In some embodiments, a motor protein for addressing in this way is a helicase, as described herein. Accordingly, in some embodiments of the disclosed methods, the polynucleotide-handling protein is modified to wholly or partially close an opening existing in at least one conformation state of the unmodified protein through which a polynucleotide strand can unbind.

The polynucleotide-handling protein may be chosen or selected according to the polynucleotide to be used in the polynucleotide-polypeptide conjugate strand and/or carrier strand used in the methods disclosed herein. Alternatively, the polynucleotide of the polynucleotide-polypeptide conjugate strand and/or carrier strand may be chosen or selected according to the polynucleotide-handling protein used to control the movement of the conjugate. For example, typically DNA motor proteins can be used when the polynucleotide is DNA. RNA motor protein can be used when the polynucleotide is RNA. Motor proteins which can process both DNA and RNA can be used when the polynucleotide is a hybrid of DNA and RNA.

In one embodiment, the motor protein is derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

In some embodiments of the claimed methods, the motor protein is a helicase, a polymerase, an exonuclease, a topoisomerase, or a variant thereof.

In one embodiment, the motor protein is an exonuclease. Suitable enzymes include, but are not limited to, exonuclease I from E. coli (SEQ ID NO: 1), exonuclease III enzyme from E. coli (SEQ ID NO: 2), RecJ from T. thermophilus (SEQ ID NO: 3) and bacteriophage lambda exonuclease (SEQ ID NO: 4), TatD exonuclease and variants thereof. Three subunits comprising the sequence shown in SEQ ID NO: 3 or a variant thereof interact to form a trimer exonuclease.

In one embodiment, the motor protein is a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase (SEQ ID NO: 5)or a variant thereof. Modified versions of Phi29 polymerase that may be used in the disclosed methods are disclosed in U.S. Pat. No. 5,576,204.

In embodiments of the methods provided herein which comprise controlling the movement of the construct, polynucleotide-polypeptide conjugate strand and/or polynucleotide carrier strand by synthesizing a strand complementary to a polynucleotide strand, the polynucleotide-handling protein is typically a polymerase, e.g. a polymerase as described herein.

In one embodiment the polynucleotide-handling protein is a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

In one embodiment the polynucleotide-handling protein is a translocase. Examples include translocases in the FtsK and SpoIII families.

In one embodiment, the polynucleotide-handling protein is a helicase. Any suitable helicase can be used in accordance with the methods provided herein. For example, the or each motor protein used in accordance with the present disclosure may be independently selected from a Hel308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep, PcrA, Pif1 and TraI. These helicases typically work on single stranded DNA. Examples of helicases that can move along both strands of a double stranded DNA include FtsK and hexameric enzyme complexes, or multisubunit complexes such as RecBCD, and are particularly suited to some embodiments disclosed herein. NS3 helicases are particularly suitable for use in the disclosed methods as they are capable of processing both DNA and RNA and so can be used in embodiments of the disclosed methods in which the target double stranded nucleic acid is a DNA-RNA hybrid.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

In one embodiment the helicase comprises the sequence shown in SEQ ID NO: 6 (Trwc Cba) or a variant thereof, the sequence shown in SEQ ID NO: 7 (Hel308 Mbu) or a variant thereof or the sequence shown in SEQ ID NO: 8 (Dda) or a variant thereof. Variants may differ from the native sequences in any of the ways discussed herein. An example variant of SEQ ID NO: 8 comprises E94C/A360C. A further example variant of SEQ ID NO: 8 comprises E94C/A360C and then (ΔM1)G1G2 (i.e. deletion of M1 and then addition of G1 and G2).

In some embodiments a motor protein (e.g. a helicase) can control the movement of the construct, polynucleotide-polypeptide conjugate strand and/or polynucleotide carrier strand in at least two active modes of operation (when the motor protein is provided with all the necessary components to facilitate movement, e.g. fuel and cofactors such as ATP and Mg²⁺ discussed herein) and one inactive mode of operation (when the motor protein is not provided with the necessary components to facilitate movement).

When provided with all the necessary components to facilitate movement (i.e. in the active modes), the motor protein (e.g. helicase) moves along the polynucleotide in a 5′ to 3′ or a 3′ to 5′ direction (depending on the motor protein). The motor protein can be used to either move the construct, polynucleotide-polypeptide conjugate strand and/or polynucleotide carrier strand away from (e.g. out of) the pore (e.g. against an applied force) or the construct, polynucleotide-polypeptide conjugate strand and/or polynucleotide carrier strand towards (e.g. into) the pore (e.g. with an applied force). For example, when the end of the construct, polynucleotide-polypeptide conjugate strand and/or polynucleotide carrier strand towards which the motor protein moves is captured by a pore, the motor protein works against the direction of the force and pulls the threaded strand out of the pore (e.g. into the cis chamber). However, when the end away from which the motor protein moves is captured in the pore, the motor protein works with the direction of the force and pushes the threaded strand into the pore (e.g. into the trans chamber).

When the motor protein (e.g. helicase) is not provided with the necessary components to facilitate movement (i.e. in the inactive mode) it can bind to the construct, polynucleotide-polypeptide conjugate strand and/or polynucleotide carrier strand and act as a brake slowing the movement of the construct, polynucleotide-polypeptide conjugate strand and/or polynucleotide carrier strand when it is moved with respect to a nanopore, e.g. by being pulled into the pore by a force. In the inactive mode, it does not matter which end of the construct, polynucleotide-polypeptide conjugate strand and/or polynucleotide carrier strand is captured, it is the applied force which determines the movement with respect to the pore, and the polynucleotide binding protein acts as a brake. When in the inactive mode, the movement control by the polynucleotide binding protein can be described in a number of ways including ratcheting, sliding and braking.

A motor protein typically requires fuel in order to handle the processing of polynucleotides. Fuel is typically free nucleotides or free nucleotide analogues. The free nucleotides may be one or more of, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are usually selected from AMP, TMP, GMP, CMP, UMP, dAMP, dTMP, dGMP or dCMP. The free nucleotides are typically adenosine triphosphate (ATP).

A cofactor for the motor protein is a factor that allows the motor protein to function. The cofactor is preferably a divalent metal cation. The divalent metal cation is preferably Mg²⁺, Mn²⁺, Ca²⁺ or Co²⁺. The cofactor is most preferably Mg²⁺.

### Nanopore

As explained above, the methods disclosed herein comprise using a polynucleotide-handling protein to control the movement of the conjugate with respect to a nanopore.

In the disclosed methods, any suitable nanopore can be used. In one embodiment a nanopore is a transmembrane pore.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

Any suitable transmembrane pore may be used in the methods provided herein. The pore may be biological or artificial. Suitable pores include, but are not limited to, protein pores, polynucleotide pores, and solid state pores.

A solid state pore may, in one embodiment, comprise a nanochannel. In some embodiments the solid state pore is a pore disclosed in WO 2003/003446, WO 2009/020682 or WO 2016/187519, each of which is incorporated by reference in their entirety.

In one embodiment, the pore may be a DNA origami pore (Langecker et al., Science, 2012; 338: 932-936). Suitable DNA origami pores are disclosed in WO2013/083983, WO 2018/011603 and WO 2020/025974, each of which is incorporated by reference in their entirety.

In one embodiment, the nanopore is a scaffolded polypeptide nanopore. In some embodiments the pore is a scaffolded polypeptide nanopore as disclosed in WO 2020/025909 or WO 2020/074399, each of which is incorporated by reference in their entirety.

In one embodiment, the nanopore is a transmembrane protein pore. A transmembrane protein pore is a polypeptide or a collection of polypeptides that permits hydrated ions, such as polynucleotide, to flow from one side of a membrane to the other side of the membrane. In the methods provided herein, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

In one embodiment, the nanopore is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

In one embodiment, the transmembrane protein pore comprises a barrel or channel through which the ions may flow. The subunits of the pore typically surround a central axis and contribute strands to a transmembrane β-barrel or channel or a transmembrane α-helix bundle or channel.

Typically, the barrel or channel of the transmembrane protein pore comprises amino acids that facilitate interaction with an analyte, such as a target polypeptide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

In one embodiment, the nanopore is a transmembrane protein pore derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and α outer membrane proteins, such as WZA and ClyA toxin.

In one embodiment the nanopore is a transmembrane pore derived from or based on Msp, α-hemolysin (α-HL), lysenin, CsgG, ClyA, Sp1 or haemolytic protein fragaceatoxin C (FraC).

In one embodiment, the nanopore is a transmembrane protein pore derived from CsgG, e.g. from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591, WO 2017/149316, WO 2017/149317, WO 2017/149318 and WO 2019/002893, each of which is hereby incorporated by reference in its entirety.

In one embodiment, the nanopore is a transmembrane pore derived from lysenin. Examples of suitable pores derived from lysenin are disclosed in WO 2013/153359, which is hereby incorporated by reference in its entirety.

In one embodiment, the nanopore is a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

In one embodiment, the nanopore is a transmembrane protein pore derived from Msp, e.g. from MspA. Examples of suitable pores derived from MspA are disclosed in WO 2012/107778.

In one embodiment, the nanopore is a transmembrane pore derived from or based on ClyA. Examples of suitable pores derived from ClyA are disclosed in Soskine et al., Nano Letters 2012 12 (9), 4895-4900; WO 2014/153625; and WO 2017/098322, each of which is hereby incorporated by reference.

In one embodiment, the nanopore is a transmembrane pore derived from Phi29. Examples of suitable pores derived from Phi29 are disclosed in Wendell et al., Nature Nanotech 4, 765-772 (2009), WO 2010/062697, WO 2019/157365 and WO 2019/157424,each of which is hereby incorporated by reference.

In some embodiments the nanopore is selected from M-ring protein, perforin-2,PlyAB (pleurotolysin), SpoIIIAG, VirB7, Type II secretion system protein D, GspD, InvG, PilQ, pentraxin, and portal proteins including T4, T7, P23_45, G20c and Phi29 nanopores.

In one embodiment, the nanopore is a transmembrane pore derived from or based on a Rhodococcus species of bacteria, for example Rhodococcus corynebacteroides or Rhodococcus ruber, for example PorARr, PorBRr or PorARc. Examples of such pores are described in Piselli et al., Eur Biophys J 51, 309-323 (2022).

As explained above, in some embodiments the nanopore comprises a constriction. The constriction is typically a narrowing in the channel which runs through the nanopore which may determine or control the signal obtained when the conjugate moves with respect to the nanopore. As used herein, both protein and solid state nanopores may comprise a “constriction”.

In some embodiments the nanopore is designed, modified or chosen to have a constriction that is sized according to the diameter of the construct. In some embodiments the pore has a constriction having a diameter of at least 1 nm, e.g. at least 1.5 nm, such as at least 2 nm, e.g. at least 2.5 nm e.g. at least 3 nm. In some embodiments the pore has a constriction having a diameter of from about 1.5 to about 2.5 nm. In some embodiments the pore has a constriction capable of translocating double-stranded DNA. Double-stranded DNA has a diameter of approximately 2 nm. A DNA-peptide chimera may be narrower or wider than duplex DNA, depending on the amino acids and strand interactions.

In some embodiments, the nanopore is modified to extend the distance between the polynucleotide-handling protein and a constriction region of the nanopore. Methods for doing so are disclosed in WO 2021/111125.

### Tags

In some embodiments of the methods provided herein, a tag on the nanopore can be used, e.g. to promote the capture of the construct by the nanopore.

The interaction between a tag on a nanopore and the binding site on the construct, polynucleotide-polypeptide conjugate strand and/or polynucleotide carrier strand (e.g., the binding site present in the polynucleotide portion of the conjugate, or in an adaptor attached to the conjugate, wherein the binding site can be provided by an anchor or a leader sequence of an adaptor or by a capture sequence within the duplex stem of an adaptor) may be reversible. For example, a polynucleotide can bind to a tag on a nanopore, e.g., via its adaptor, and release at some point, e.g., during characterization of the polynucleotide by the nanopore and/or during processing by a motor protein. A strong non-covalent bond (e.g., biotin/avidin) is still reversible and can be useful in some embodiments of the methods described herein. For example, a pair of pore tag and polynucleotide adaptor can be designed to provide a sufficient interaction between the complement of a double stranded polynucleotide (or a portion of an adaptor that is attached to the complement) and the nanopore such that the complement is held close to the nanopore (without detaching from the nanopore and diffusing away) but is able to release from the nanopore as it is processed.

A pore tag and polynucleotide adaptor can be configured such that the binding strength or affinity of a binding site on the polynucleotide (e.g., a binding site provided by an anchor or a leader sequence of an adaptor or by a capture sequence within the duplex stem of an adaptor) to a tag on a nanopore is sufficient to maintain the coupling between the nanopore and polynucleotide until an applied force is placed on it to release the bound polynucleotide from the nanopore.

In some embodiments, the tags or tethers are uncharged. This can ensure that the tags or tethers are not drawn into the nanopore under the influence of a potential difference if present.

One or more molecules that attract or bind the construct, polynucleotide-polypeptide conjugate strand and/or polynucleotide carrier strand may be linked to the nanopore. Any molecule that hybridizes to the conjugate, adaptor and/or polynucleotide may be used. The molecule attached to the pore may be selected from a PNA tag, a PEG linker, a short oligonucleotide, a positively charged amino acid and an aptamer. Pores having such molecules linked to them are known in the art. For example, pores having short oligonucleotides attached thereto are disclosed in Howarka et al (2001) Nature Biotech. 19: 636-639 and WO 2010/086620, and pores comprising PEG attached within the lumen of the pore are disclosed in Howarka et al (2000) J. Am. Chem. Soc. 122(11): 2411-2416.

A short oligonucleotide attached to the nanopore, which comprises a sequence complementary to a sequence in the conjugate (e.g. in a leader sequence or another single stranded sequence in an adaptor) may be used to enhance capture of the construct, polynucleotide-polypeptide conjugate strand and/or polynucleotide carrier strand in the methods described herein.

In some embodiments, the tag or tether may comprise or be an oligonucleotide (e.g., DNA, RNA, LNA, BNA, PNA, or morpholino). The oligonucleotide can have about 10-30 nucleotides in length or about 10-20 nucleotides in length. In some embodiments, the oligonucleotide can have at least one end (e.g., 3′- or 5′-end) modified for conjugation to other modifications or to a solid substrate surface including, e.g., a bead. The end modifiers may add a reactive functional group which can be used for conjugation. Examples of functional groups that can be added include, but are not limited to amino, carboxyl, thiol, maleimide, aminooxy, and any combinations thereof. The functional groups can be combined with different length of spacers (e.g., C3, C9, C12, Spacer 9 and 18) to add physical distance of the functional group from the end of the oligonucleotide sequence.

Examples of modifications on the 3′ and/or 5′ end of oligonucleotides include, but are not limited to 3′ affinity tag and functional groups for chemical linkage (including, e.g., 3′-biotin, 3′-primary amine, 3′-disulfide amide, 3′-pyridyl dithio, and any combinations thereof); 5′ end modifications (including, e.g., 5′-primary ammine, and/or 5′-dabcyl), modifications for click chemistry (including, e.g., 3′-azide, 3′-alkyne, 5′-azide, 5′-alkyne), and any combinations thereof.

In some embodiments, the tag or tether may further comprise a polymeric linker, e.g., to facilitate coupling to the nanopore. An exemplary polymeric linker includes, but is not limited to polyethylene glycol (PEG). The polymeric linker may have a molecular weight of about 500 Da to about 10 kDa (inclusive), or about 1 kDa to about 5 kDa (inclusive). The polymeric linker (e.g., PEG) can be functionalized with different functional groups including, e.g., but not limited to maleimide, NHS ester, dibenzocyclooctyne (DBCO), azide, biotin, amine, alkyne, aldehyde, and any combinations thereof.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of a nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444.

In some embodiments where one or more tags or tethers are attached to a nanopore via cysteine linkage(s), the one or more cysteines can be introduced to one or more monomers that form the nanopore by substitution. In some embodiments, the nanopore may be chemically modified by attachment of (i) Maleimides including diabromomaleimides such as: 4-phenylazomaleinanil, 1.N-(2-Hydroxyethyl)maleimide, N-Cyclohexylmaleimide, 1.3-Maleimidopropionic Acid, 1.1-4-Aminophenyl-1H-pyrrole,2,5,dione, 1.1-4-Hydroxyphenyl-1H-pyrrole,2,5,dione, N-Ethylmaleimide, N-Methoxycarbonylmaleimide, N-tert-Butylmaleimide, N-(2-Aminoethyl)maleimide, 3-Maleimido-PROXYL, N-(4-Chlorophenyl)maleimide, 1-[4-(dimethylamino)-3,5-dinitrophenyl]-1H-pyrrole-2,5-dione, N-[4-(2-Benzimidazolyl)phenyl]maleimide, N-[4-(2-benzoxazolyl)phenyl]maleimide, N-(1-naphthyl)-maleimide, N-(2,4-xylyl)maleimide, N-(2,4-difluorophenyl)maleimide, N-(3-chloro-para-tolyl)-maleimide, 1-(2-amino-ethyl)-pyrrole-2,5-dione hydrochloride, 1-cyclopentyl-3-methyl-2,5-dihydro-1H-pyrrole-2,5-dione, 1-(3-aminopropyl)-2,5-dihydro-1H-pyrrole-2,5-dione hydrochloride, 3-methyl-1-[2-oxo-2-(piperazin-1-yl)ethyl]-2,5-dihydro-1H-pyrrole-2,5-dione hydrochloride, 1-benzyl-2,5-dihydro-1H-pyrrole-2,5-dione, 3-methyl-1-(3,3,3-trifluropropyl)-2,5-dihydro-1H-pyrrole-2,5-dione, 1-[4-(methylamino)cyclohexyl]-2,5-dihydro-1H-pyrrole-2,5-dione trifluroacetic acid, SMILES O═C1C═CC(═O)N1CC=2C═CN═CC2, SMILES O═C1C═CC(═O)N1CN2CCNCC2, 1-benzyl-3-methyl-2,5-dihydro-1H-pyrrole-2,5-dione, 1-(2-fluorophenyl)-3-methyl-2,5-dihydro 1H-pyrrole-2,5-dione, N-(4-phenoxyphenyl)maleimide, N-(4-nitrophenyl)maleimide (ii) Iodocetamides such as: 3-(2-Iodoacetamido)-proxyl, N-(cyclopropylmethyl)-2-iodoacetamide, 2-iodo-N-(2-phenylethyl)acetamide, 2-iodo-N-(2,2,2-trifluoroethyl)acetamide, N-(4-acetylphenyl)-2-iodoacetamide, N-(4-(aminosulfonyl)phenyl)-2-iodoacetamide, N-(1,3-benzothiazol-2-yl)-2-iodoacetamide, N-(2,6-diethylphenyl)-2-iodoacetamide, N-(2-benzoyl-4-chlorophenyl)-2-iodoacetamide, (iii) Bromoacetamides: such as N-(4-(acetylamino)phenyl)-2-bromoacetamide, N-(2-acetylphenyl)-2-bromoacetamide, 2-bromo-n-(2-cyanophenyl)acetamide, 2-bromo-N-(3-(trifluoromethyl)phenyl)acetamide, N-(2-benzoylphenyl)-2-bromoacetamide, 2-bromo-N-(4-fluorophenyl)-3-methylbutanamide, N-Benzyl-2-bromo-N-phenylpropionamide, N-(2-bromo-butyryl)-4-chloro-benzenesulfonamide, 2-Bromo-N-methyl-N-phenylacetamide, 2-bromo-N-phenethyl-acetamide,2-adamantan-1-yl-2-bromo-N-cyclohexyl-acetamide, 2-bromo-N-(2-methylphenyl)butanamide, Monobromoacetanilide, (iv) Disulphides such as: aldrithiol-2,aldrithiol-4, isopropyl disulfide, 1-(Isobutyldisulfanyl)-2-methylpropane, Dibenzyl disulfide, 4-aminophenyl disulfide, 3-(2-Pyridyldithio)propionic acid, 3-(2-Pyridyldithio)propionic acid hydrazide, 3-(2-Pyridyldithio)propionic acid N-succinimidyl ester, am6amPDP1-PCD and (v) Thiols such as: 4-Phenylthiazole-2-thiol, Purpald, 5,6,7,8-tetrahydro-quinazoline-2-thiol.

In some embodiments, the tag or tether may be attached directly to a nanopore or via one or more linkers. The tag or tether may be attached to the nanopore using the hybridization linkers described in WO 2010/086602. Alternatively, peptide linkers may be used. Peptide linkers are amino acid sequences. The length, flexibility and hydrophilicity of the peptide linker are typically designed such that it does not to disturb the functions of the monomer and pore. Preferred flexible peptide linkers are stretches of 2 to 20, such as 4, 6, 8, or 16, serine and/or glycine amino acids. More preferred flexible linkers include (SG)i, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

### Membrane

Typically, in the disclosed methods, the nanopore is typically present in a membrane. Any suitable membrane may be used in the system.

The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

In some embodiments, the membrane is one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In some embodiments, a lipid bilayer is formed as described in International Application No. WO 2009/077734. Advantageously in this method, the lipid bilayer is formed from dried lipids. In some embodiments, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn- Glycero-3-Phosphocholine; and ceramides.

In another embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The methods disclosed herein are typically carried out using (i) an artificial amphiphilic layer comprising a pore, (ii) an isolated, naturally-occurring lipid bilayer comprising a pore, or (iii) a cell having a pore inserted therein. The methods are typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The disclosed methods are typically carried out in vitro.

### Conditions

The disclosed characterisation methods may be carried out using any apparatus that is suitable for investigating a membrane/pore system in which a pore is inserted into a membrane. The characterisation method may be carried out using any apparatus that is suitable for transmembrane pore sensing. For example, the apparatus may comprise a chamber comprising an aqueous solution and a barrier that separates the chamber into two sections. The barrier may have an aperture in which a membrane containing a transmembrane pore is formed. Transmembrane pores are described herein.

The characterisation methods may be carried out using the apparatus described in WO 2008/102120, WO 2010/122293 or WO 00/28312.

The characterisation methods may comprise optical measurements, for example such as described in WO 2016/009180 and WO 2021/198695.

The characterisation methods may involve measuring the ion current flow through the pore, typically by measurement of a current. Alternatively, the ion flow through the pore may be measured optically, such as disclosed by Heron et al: J. Am. Chem. Soc. 9 Vol. 131, No. 5, 2009. Therefore the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The characterisation methods may be carried out on a silicon-based array of wells where each array comprises 128, 256, 512, 1024, 2000, 3000, 4000, 6000, 10000, 12000, 15000 or more wells.

The characterisation methods may involve the measuring of a current flowing through the pore. The method is typically carried out with a voltage applied across the membrane and pore. The voltage used is typically from +2 V to−2 V, typically −400 mV to +400 mV. The voltage used is preferably in a range having a lower limit selected from −400 mV, −300 mV,−200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The characterisation methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt may be an alkaline earth metal salt such as calcium chloride (CaCl₂). The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The characterisation method may be carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of binding/no binding to be identified against the background of normal current fluctuations.

The characterisation methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used may be about 7.5.

The characterisation methods may be carried out at from 0 °C to 100 °C, from 15 °C to 95 °C, from 16 °C to 90 °C, from 17 °C to 85 °C, from 18 °C to 80 °C, 19 °C to 70 °C, or from 20 °C to 60 °C. The characterisation methods are typically carried out at room temperature. The characterisation methods are optionally carried out at a temperature that supports enzyme function, such as about 37 °C.

### System

Also provided is a system comprising
a construct comprising (i) a polynucleotide-polypeptide conjugate strand comprising a target polypeptide conjugated at each end of said target polypeptide to one or more polynucleotide flanking strands and (ii) a polynucleotide carrier strand;
a nanopore capable of co-translocating the polynucleotide-polypeptide conjugate strand and the polynucleotide flanking strand of the construct; and
a polynucleotide-handling protein.

In some embodiments the construct, nanopore, and/or polynucleotide-handling protein are as described in more detail herein.

Also provided is a kit comprising:
a nanopore;
a first polynucleotide comprising a reactive functional group for conjugating to a first end of a target polypeptide;
a second polynucleotide comprising a reactive functional group for conjugating to a second end of the target polypeptide; and
a polynucleotide-handling protein.

In some embodiments the nanopore and polynucleotide-handling protein are each as described in more detail herein. In some embodiments the first and second polynucleotides are each flanking strands as described herein.

The system and kit may each independently be configured for use with an algorithm, also provided herein, adapted to be run on a computer system. The algorithm may be adapted to detect information characteristic of a polypeptide (e.g. characteristic of the sequence of the polypeptide and/or whether the polypeptide is modified), and to selectively process the signal obtained as a construct comprising the polypeptide conjugated to a polynucleotide flanking strand and hybridised to a polynucleotide carrier strand moves with respect to the nanopore (i.e. as the strands co-translocate the pore).

In some embodiments a system comprises computing means configured to detect information characteristic of a polypeptide (e.g. characteristic of the sequence of the polypeptide and/or whether the polypeptide is modified) and to selectively process the signal obtained as a conjugate comprising the polypeptide conjugated to a polynucleotide flanking strand and a polynucleotide carrier strand co-translocate the nanopore. In some embodiments the system comprises receiving means for receiving data from detection of the polypeptide, processing means for processing the signal obtained as the conjugate moves with respect to the nanopore, and output means for outputting the characterisation information thus obtained.

### Characterising a Target Polynucleotide Sequence

The methods discussed above may also be applied to the characterisation of polynucleotide strands, which may for example be those which do not comprise a polypeptide sequence. Those skilled in the art will appreciate that many of the advantages set out for the methods disclosed above which refer to characterisation of and movement of polynucleotide-polypeptide constructs apply equally to methods in which the construct being assessed does not comprise a polypeptide. For example, in some embodiments the target polypeptide portion of a construct as described herein may be exchanged for a target polynucleotide sequence, and the methods disclosed above may accordingly be applied to characterise the target polynucleotide sequence. In such embodiments, the features described above may be generally applied unless indicated otherwise by the context. Thus, for example, the polynucleotide strands and their formation, the construct and its formation, the polynucleotide-handling protein, the nanopore, and the general set-up and movement schemes may all be as described above. This is illustrated further below.

Accordingly, provided is a method of characterising a target polynucleotide sequence, comprising
contacting (i) a target polynucleotide strand comprising the target polynucleotide sequence with (ii) a polynucleotide carrier strand, thereby forming a double-stranded polynucleotide construct;
contacting the construct with a nanopore under conditions such that both the target polynucleotide strand and the polynucleotide carrier strand co-translocate through the nanopore; and
taking one or more measurements characteristic of the target polynucleotide as the construct moves with respect to the nanopore,
   thereby characterising the target polynucleotide.

In some embodiments the target polynucleotide sequence is conjugated at each end of said target polynucleotide sequence to one or more polynucleotide flanking strands. In some embodiments said one or more polynucleotide flanking strands are each independently complementary to a region of the polynucleotide carrier strand. In some embodiments the one or more polynucleotide flanking strands are each independently at least partially hybridized to the polynucleotide carrier strand.

In some embodiments the target polynucleotide sequence is complementary to a region of the polynucleotide carrier strand. Thus, in some embodiments, in the construct formed by the target polynucleotide strand and the polynucleotide carrier strand, the target polynucleotide strand and the polynucleotide carrier strand are hybridised together in the region of the target polynucleotide sequence.

In some embodiments the target polynucleotide sequence is non-complementary to the polynucleotide carrier strand. Thus, in some embodiments, in the construct formed by the target polynucleotide strand and the polynucleotide carrier strand, the target polynucleotide strand and the polynucleotide carrier strand are not hybridised together in the region of the target polynucleotide. In some embodiments the target polynucleotide strand and the polynucleotide carrier strand are hybridised together in the region of the polynucleotide flanking strands but are not hybridised together in the region of the target polynucleotide sequence.

Also provided, therefore, is a method of characterising a target polynucleotide sequence, comprising
contacting (i) a target polynucleotide strand comprising a target polynucleotide sequence attached to a polynucleotide flanking strand with (ii) a polynucleotide-handling protein capable of controlling the movement of the polynucleotide flanking strand with respect to a nanopore; and
contacting the target polynucleotide strand with a nanopore under conditions such that the polynucleotide-handling protein controls the movement of the target polynucleotide strand with respect to the nanopore; and
taking one or more measurements characteristic of the target polynucleotide sequence as the polynucleotide flanking strand and the target polynucleotide analyte co-translocate through the nanopore,
   thereby characterising the target polypeptide.

In some embodiments, the polynucleotide flanking strand is at least partially hybridized to a polynucleotide carrier strand prior to such methods.

In some embodiments of the above methods, the target polynucleotide strand comprises a plurality of target polynucleotide sequences. For example, in some embodiments the or each target polynucleotide sequence may independently have a length of from about 5 to about 1000 nucleotide units. The polynucleotide strands used in the above methods may be any of the polynucleotide strands described in more detail herein.

In some embodiments the target polynucleotide sequences comprises different types of nucleotide to the polynucleotide carrier strand and/or polynucleotide flanking strand. For example, the target polynucleotide sequence may comprise or consist of RNA nucleotides and the polynucleotide carrier strand and/or flanking strand may comprise or consist of DNA nucleotides. In some embodiments the target polynucleotide sequence may comprise or consist of DNA nucleotides and the polynucleotide carrier strand and/or flanking strand may comprise or consist of RNA nucleotides. In some embodiments the target polynucleotide sequences comprises the same type of nucleotides as the polynucleotide carrier strand and/or polynucleotide flanking strand. For example, the target polynucleotide sequence may comprise or consist of DNA nucleotides and the polynucleotide carrier strand and/or flanking strand may comprise or consist of DNA nucleotides.

The target polynucleotide strand, polynucleotide carrier strand and/or construct may be mechanically manipulated in the manner discussed above in the context of polynucleotide-polypeptide conjugate strands. Accordingly, the above methods may comprise mechanically manipulating the construct, target polynucleotide strand and/or polynucleotide carrier strand, thereby moving said construct, target polynucleotide strand and/or polynucleotide carrier strand with respect to the nanopore. The movement may be in a direction opposite to a potential applied across said nanopore, such as a voltage potential applied across said nanopore.

The above methods may comprise contacting the construct or a polynucleotide strand thereof with a polynucleotide-handling protein capable of controlling the movement of one or more strands of the construct (e.g. the one or more polynucleotide flanking strands and/or the target polynucleotide strand), thereby controlling the movement of the construct with respect to the nanopore. As discussed herein in more detail, in some embodiments the polynucleotide-handling protein controls the movement of both strands of the construct. In some embodiments the polynucleotide-handling protein controls the movement of just one of the strands of the construct, e.g. the target polynucleotide strand or the polynucleotide carrier strand.

The polynucleotide-handling protein may be located on either the cis or trans side of the nanopore, and may control the movement of the construct in the direction from the cis side of the nanopore to the trans side of the nanopore, or from the trans side to the cis side of the nanopore, as described in more detail herein in the context of methods that involve the movement of and characterisation of polynucleotide-polypeptide strands and constructs comprising them.

The polynucleotide-handling protein may be any of the polynucleotide-handling proteins described in more detail herein. In some embodiments, prior to the above methods the polynucleotide-handling protein is bound to the polynucleotide carrier strand in a region of the polynucleotide carrier strand that is spanned by a non-hybridised region of a polynucleotide flanking strand. In some embodiments the polynucleotide-handling protein is capable of remaining bound to the construct as described in more detail herein. For example, the polynucleotide-handling protein may be modified to prevent it from disengaging from the construct, target polynucleotide strand and/or polynucleotide carrier strand. The polynucleotide-handling protein may be modified in any manner as described herein, such as by being modified to wholly or partially close an opening existing in at least one conformation state of the unmodified protein through which a polynucleotide strand can unbind. As discussed in more detail herein, in some embodiments the polynucleotide-handling protein is or comprises a helicase, translocase or helicase-nuclease complex.

As discussed in more detail herein in the context of methods that involve the movement of and characterisation of polynucleotide-polypeptide strands and constructs comprising them, in some embodiments of the methods discussed above the construct comprises a stalling moiety which may be positioned such that the stalling moiety is located between the polynucleotide-handling protein and the target polynucleotide sequence prior to the methods. Adapters, tethers, anchors and/or blocking moieties may be further comprised in the constructs, as discussed above in more detail.

As discussed in more detail herein in the context of methods that involve the movement of and characterisation of polynucleotide-polypeptide strands and constructs comprising them, in some embodiments of the methods discussed above, the construct or one or more of its constituent strands may be “flossed” through the nanopore by carrying out the method such that (i) the strand(s) translocate the nanopore in a first direction with respect to the nanopore; (ii) the strand(s) are allowed to move in a direction opposite to the direction of movement with respect to the nanopore in step (i); (iii) the strand(s) are optionally allowed to move in the first direction; and steps (ii) and (iii) are optionally repeated to oscillate the strand(s) through the nanopore.

The characteristics that may be measured in the above methods may include, for example, (i) the length of the target polynucleotide sequence, (ii) the identity of the target polynucleotide sequence, (iii) the sequence of the target polynucleotide sequence, (iv) the secondary structure of the target polynucleotide sequence and (v) whether or not the target polynucleotide sequence is modified.

The nanopore may be any of the nanopores discussed herein, such as a protein nanopore, e.g. a β-barrel protein nanopore.

It is to be understood that although particular embodiments, specific configurations as well as materials and/or molecules, have been discussed herein for methods according to the present invention, various changes or modifications in form and detail may be made without departing from the scope and spirit of this invention. The preceding embodiments and following examples are provided for illustration only, and should not be considered limiting the application. The application is limited only by the claims.

### EXAMPLES

### Example 1

This example demonstrates discrimination between three different peptides each comprised in a construct comprising a peptide analyte and a polynucleotide strand, such as described herein above.

Discrimination was demonstrated using a modified transmembrane protein nanopore derived from Rhodococcus. The internal diameter of the nanopore was wide enough to accommodate double-strand DNA (dsDNA). The pore was formed from monomers with the following amino acid sequence:

(SEQ ID NO: 9)
MAVDDSNSVVDGGGNTITVSQSDTFINSVFPLDGSPLTREWFHNG
RAIVDVTGPDAEDFSGTVTIGYQVGYPASLGGRLTFSYTTPGLNL
SVGNGVAATVTNVLPQAGVGVTLTPGPGIETVAVASGAASGAHTE
IQIANLHGTATKIAGNVSVRPYVQVVSSNGDVATTFGQPWRFNGS
GGENLYFQGSGSGSAWSHPQFEK.

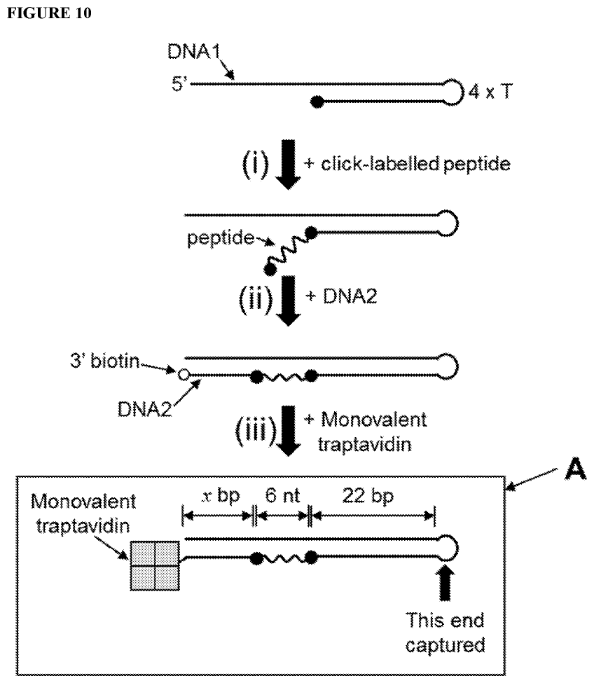

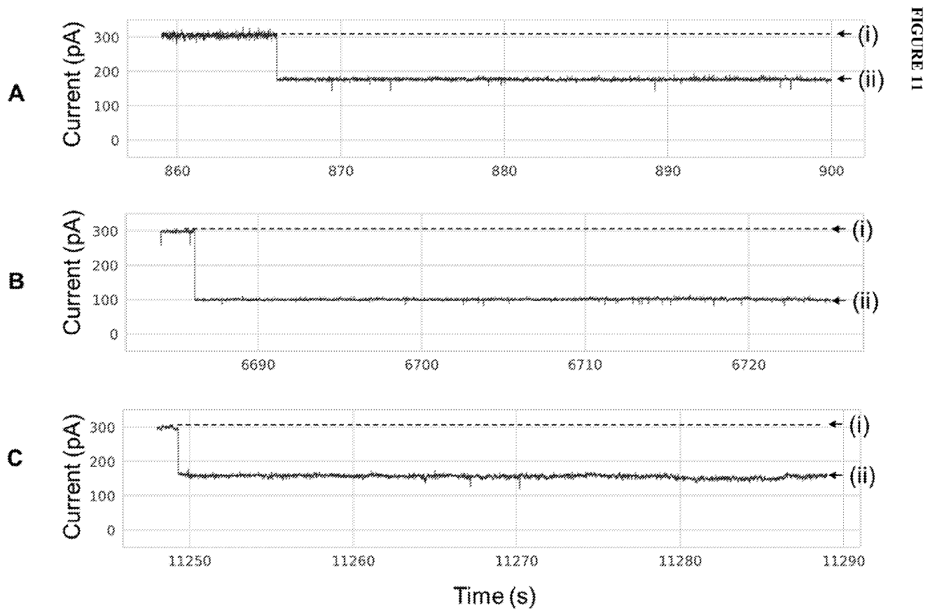

The peptide was conjugated between two dsDNA oligonucleotides, with a single-stranded DNA (ssDNA) strand acting as a “carrier” strand, and a terminal biotin to prevent the full translocation of the conjugate through the nanopore. To simulate the stepwise movement of this conjugate through the nanopore, a series of conjugates was prepared in which the distance in dsDNA base-pairs between the terminal biotin and the peptide was varied (distance “x”, as defined in FIG. 10).

### Construct Assembly

All constructs were formed of two DNA oligonucleotides (DNA1 and DNA2) and a peptide, as shown in FIG. 10, wherein DNA1 bears a hairpin and 3′ terminal TCO group, the peptide bears N-terminal azide and C-terminal methyltetrazine groups, and DNA2 bears a 5′ BCN group and 3′ biotin group. Peptides were synthesised with the sequence N-GGSGXXSGSG-C, in which the middle two amino acid residues (XX) were varied: DD, RR or YY, and modified with N-terminal azide, and C-terminal methyltetrazine connected via an ethylenediamine linker.

SPRI bead mix was prepared by exchanging SpeedBead magnetic carboxylate modified particles (Merck, cat #GE65152105050250) into SPRI wash buffer (25 mM Tris-Cl (pH 7.5), 2.5 M NaCl, 28% (w/v) PEG-8000) at a final concentration of 0.25% (w/v).

Constructs were assembled in a two-stage process with click reactions proceeding sequentially. DNA1 oligonucleotide (1 μM) was first reacted with 200 μM peptide in 25 mM HEPES-NaOH (pH 7.5), 500 mM NaCl, 0.01% (v/v) Tween-20. Reactions were covered with foil and incubated at 37 °C for 2 hours with shaking at 800 rpm. Reactions were purified as follows: 3.7-fold excess of SPRI bead mix was added; mixtures were incubated for 5 minutes at room temperature with shaking on a Hula mixer; beads were pelleted and washed twice with SPRI wash buffer, then eluted in 25 mM HEPES-NaOH (pH 7.5), 50 mM NaCl. Samples were heated to 95 °C for 3 mins and then snap-cooled on ice and left for at least 10 mins before further processing.

A second click reaction was then assembled consisting of 1 μM peptide-DNA conjugate (the product of the first click reaction) and 4 μM BCN-modified DNA strand (DNA2) with 3.7-fold excess of SPRI wash buffer. Reactions were incubated at 37 °C for 2 hours with shaking at 1500 rpm. Reactions were purified as per following the first click reaction to yield a polynucleotide-polypeptide conjugate. Assemblies were analysed by denaturing and non-denaturing PAGE. As a control, a conjugate was prepared by annealing two oligonucleotides together, one of which carried a 3′ terminal biotin group. All sequences of oligonucleotides used in this Example are listed in Table 3.

### Data Acquisition

Electrical data were collected using MinION flow cells (Oxford Nanopore Technologies plc) comprising modified Rhodococcus pores as described above.

Samples were prepared by incubating an equimolar amount of monovalent traptavidin with the polynucleotide-polypeptide conjugate in Running Buffer (25 mM HEPES-KOH (pH 8.0), 500 mM KCl). Flow cells were first flushed with at least 1 mL of Running Buffer. Either Running Buffer alone or Running Buffer containing each sample were sequentially added to the flow cell via the SpotON port, with flushes of Running Buffer between each sample addition. Data were acquired with a sample rate of 5 kHz and applied potential of 180 mV, with a reversal potential of 120 mV applied at one-minute intervals to eject each conjugate from the nanopore. The data were analysed by determining the normalised blockade level caused by the sample with respect to the open pore level.

### Results and Conclusions

FIG. 11 shows raw data collected from a series of samples in which the distance between the monovalent traptavidin and the polypeptide was 11 bp and the polypeptide sequence (read N-to-C) was GGSGDDSGSG (SEQ ID NO: 10), GGSGRRSGSG (SEQ ID NO: 11) or GGSGYYSGSG (SEQ ID NO: 12). Each example shows an open pore current of ~275 pA followed by a blockade level unique to the peptide as the analyte is capture in the nanopore. This data shows that the nanopore can discriminate between the three peptides tested.

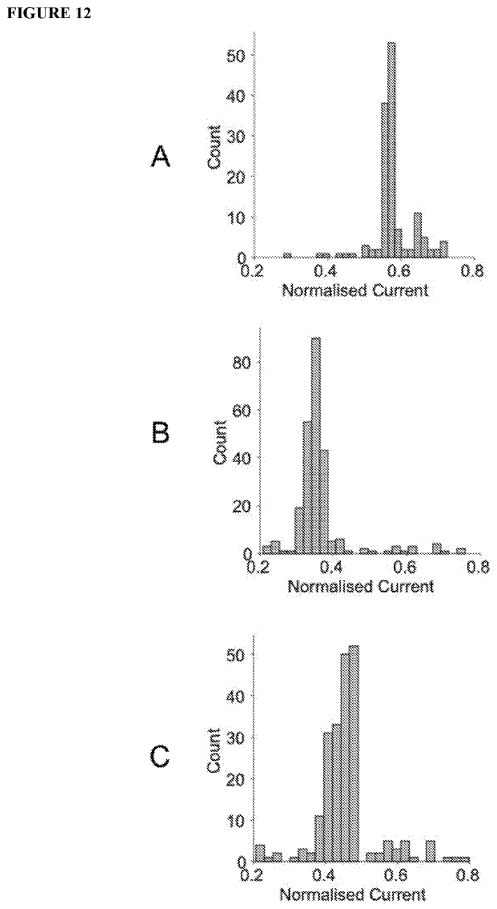

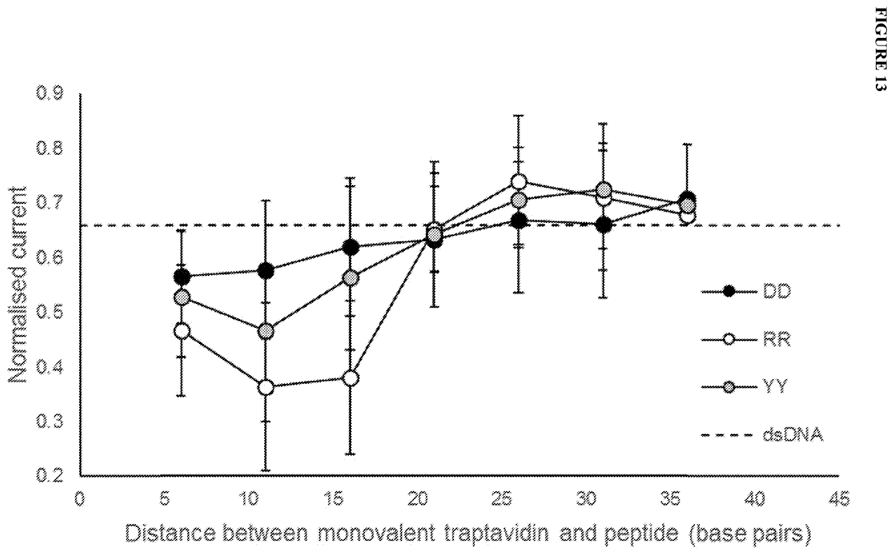

FIG. 12 shows histograms of the normalised blockade levels for a series of analytes in which the distance between monovalent traptavidin was 11 bp, and the peptide sequence was either GGSGDDSGSG (SEQ ID NO: 10), GGSGRRSGSG (SEQ ID NO: 11) or GGSGYYSGSG (SEQ ID NO: 12).

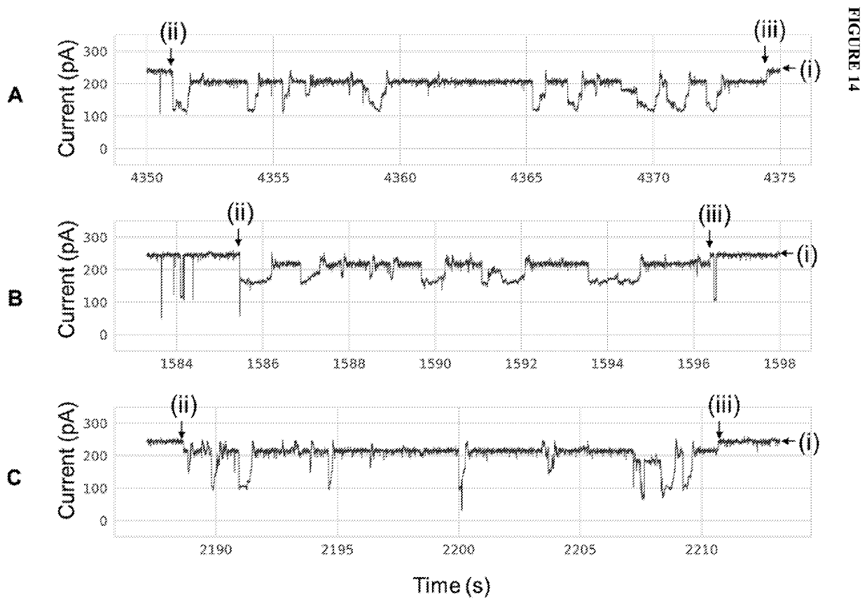

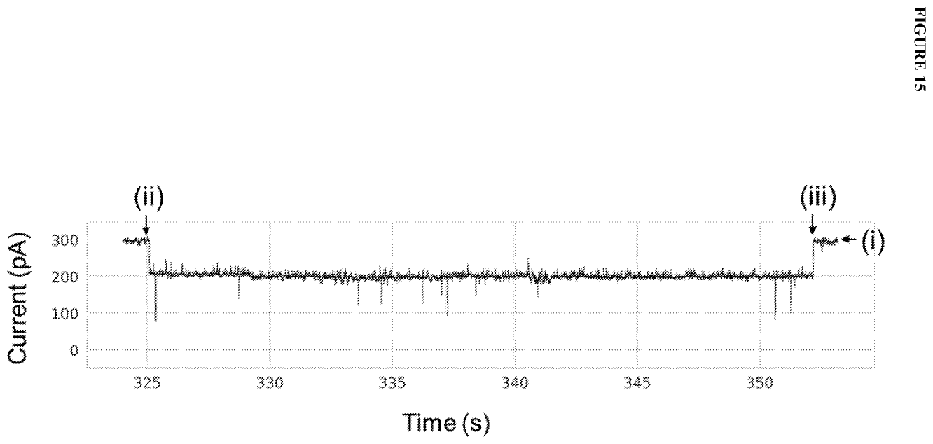

FIG. 13 shows the aggregated data for distances of 6 bp to 36 bp in 5 bp increments, represented as a plot of normalised current (I/I₀) against the distance between monovalent traptavidin and the peptide. The data show that 11 bp is the most sensitive position for the discrimination of peptides using this nanopore. At distances of 21 bp or greater of the peptide from monovalent traptavidin, the blockade level was indistinguishable from dsDNA alone. The data demonstrate that the stepwise movement of polynucleotide-polypeptide conjugates through a nanopore can generate a distinctive current-distance trace that is dependent on the peptide analysed.

TABLE 3
oligonucleotides used in Example 1.
3 denotes an amino C3 labelled with TCO.
2 denotes a 5′ amino C3 labelled with BCN,
1 denotes a 3′ Biotin modification.
“+” before a base denotes LNA.

| Distance between | | |
|---|---|---|
| monovalent | | DNA2 sequence |
| traptavidin and | DNA1 sequence | (as defined |
| peptide (bp) | (as defined in FIG. 1) | in FIG. 1) |
| 6 | +G+T+C+C+A+CTTTTTTGCTGCGGCTTAGCT | 2GTGGAC1 |
| | GTGATAGCTTTTGCTATCACAGCTAAGCCGC | (SEQ ID NO: 20) |
| | AGC3 | |
| | (SEQ ID NO: 13) | |
| 11 | GTCCATTATTCTTTTTTGCTGCGGCTTAGCT | 2GAATAATGGAC1 |
| | GTGATAGCTTTTGCTATCACAGCTAAGCCGC | (SEQ ID NO: 21) |
| | AGC3 | |
| | (SEQ ID NO: 14) | |
| 16 | GTATTGTCCATTATTCTTTTTTGCTGCGGCTT | 2GAATAATGGACAATAC1 |
| | AGCTGTGATAGCTTTTGCTATCACAGCTAAG | (SEQ ID NO: 22) |
| | CCGCAGC3 | |
| | (SEQ ID NO: 15) | |
| 21 | GTATTGTATTGTCCATTATTCTTTTTTGCTGC | 2GAATAATGGACAATAC |
| | GGCTTAGCTGTGATAGCTTTTGCTATCACAG | AATAC1 |
| | CTAAGCCGCAGC3 | (SEQ ID NO: 23) |
| | (SEQ ID NO: 16) | |
| 26 | GTATTGTATTGTATTGTCCATTATTCTTTTTT | 2GAATAATGGACAATACA |
| | GCTGCGGCTTAGCTGTGATAGCTTTTGCTAT | ATACAATACI |
| | CACAGCTAAGCCGCAGC3 | (SEQ ID NO: 24) |
| | (SEQ ID NO: 17) | |
| 31 | GTATTGTATTGTATTGTATTGTCCATTATTCT | 2GAATAATGGACAATACA |
| | TTTTTGCTGCGGCTTAGCTGTGATAGCTTTT | ATACAATACAATAC1 |
| | GCTATCACAGCTAAGCCGCAGC3 | (SEQ ID NO: 25) |
| | (SEQ ID NO: 18) | |
| 36 | GTATTGTATTGTATTGTATTGTATTGTCCAT | 2GAATAATGGACAATACA |
| | TATTCTTTTTTGCTGCGGCTTAGCTGTGATA | ATACAATACAATACAATA |
| | GCTTTTGCTATCACAGCTAAGCCGCAGC3 | C1 |
| | (SEQ ID NO: 19) | (SEQ ID NO: 26) |

### Example 2

This example demonstrates the repeated movement of a polynucleotide-polypeptide conjugate through a nanopore with an internal diameter wide enough to accommodate double-strand DNA (dsDNA). The peptide was conjugated between two dsDNA oligonucleotides, with single-stranded DNA (ssDNA) acting as “carrier” strand.

### Construct Assembly

The sequences of oligonucleotides used are outlined in Table 4. Three constructs were generated that differed based on the polypeptide sequence used. Peptides were synthesised with the sequence N-GGSGXXSGSG-C, in which the middle two amino acid residues (XX) were varied: DD, RR or YY, and modified with N-terminal azide, and C-terminal methyltetrazine connected via an ethylenediamine linker. Constructs used in this Example were assembled according to the method described in Example 1. The constructs differ from those used in Example 1 in that they bear a 3′ poly(dA) tail instead of terminal biotin, which aids the loading of the helicase from solution.

### Data Acquisition

Electrical data were collected using MinION flow cells (Oxford Nanopore Technologies plc) comprising modified Rhodococcus pores as used in Example 1. Samples were prepared by incubating 0.2 μM of polynucleotide-polypeptide conjugate with 1 μM wild-type Hel308 helicase in either Running Buffer (25 mM HEPES-KOH (pH 8.0), 500 mM KCl) or Sequencing Buffer (25 mM HEPES-KOH (pH 8.0), 350 mM KCl, 50 mM ATP, 50 mM MgCl₂, 2.2 mM EDTA). The Running Buffer lacks ATP and was performed as a control for ATP-dependent enzymatic movement. Flow cells were first flushed with at least 1 mL of Running Buffer or Sequencing Buffer. Samples (75 μL) were then introduced into the flow cell via the SpotON port. Electrical data were acquired with a sample rate of 5 kHz and applied potential of 180 mV, with a reversal potential of 120 mV applied at one-minute intervals to eject each conjugate from the nanopore.

### Results and Discussion

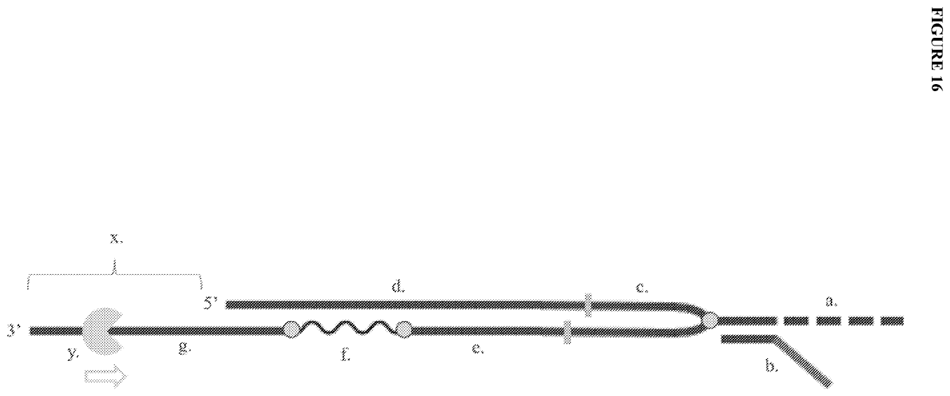

FIG. 14 shows example traces of enzymatically-driven movement of the polypeptide-polynucleotide conjugate using the helicase. The experiments were performed under conditions of excess helicase with respect to conjugate. The presence of repetitive traces shows that the conjugates are captured via the hairpin end. Bound helicase prevents the conjugate fully translocating, then the helicase moves 3′-5′ along ssDNA to control the movement of dsDNA out of the nanopore. As the helicase approaches the junction between ssDNA and peptide, it dissociates from ssDNA and the position of the conjugate in the nanopore drops back rapidly to a second helicase, bound 3′-distal to the enzyme, that dissociates, so the enzymes repetitively shuttle the conjugate through the nanopore. The movement is ATP-dependent, as demonstrated by the controls lacking ATP (FIG. 15) which show only blocks and not repetitive patterns. The data show that it is possible to repetitively move a polypeptide-polynucleotide conjugate using a helicase through a nanopore.

TABLE 4
oligonucleotides used in Example 2.
3 denotes an amino C3 labelled with TCO.
2 denotes a 5′ amino C3 labelled with BCN.
“+” before a base denotes LNA.

| DNA1 sequence | DNA2 sequence |
|---|---|
| (as defined in FIG. 1) | (as defined in FIG. 1) |
| +G+T+C+C+ATTATTCTTTTT | 2GAATAATGGACAAAAAAA |
| TGCTGCGGCTTAGCTGTGATA | AAAAAAAAAAAAAAAAAAA |
| GCTTTTGCTATCACAGCTAAG | AAA |
| CCGCAGC3 | (SEQ ID NO: 28) |
| (SEQ ID NO: 27) | |

### Example 3

This example demonstrates the repeated movement of a polynucleotide-polypeptide conjugate through a nanopore. The conjugate is moved in the direction ‘out’ of the nanopore by a Hel308 helicase enzyme. The peptide is conjugated between two dsDNA oligonucleotides.

The conjugate contains a single-stranded overhang onto which a helicase is loaded from solution. Bound helicase prevents the conjugate fully translocating through the nanopore; the helicase moves in the direction 3′ to 5′ along a single strand of DNA and controls the movement of dsDNA and peptide in the direction ‘out’ of the nanopore. As the helicase approaches a junction in the construct between DNA and peptide, it dissociates from the single strand of DNA and the position of the conjugate in the nanopore drops back rapidly to a second helicase, which has loaded onto the construct from solution at a position 3′-distal to the first helicase. The cycle then repeats, with the second helicase again controlling the movement of the double-stranded portion of the construct in the direction ‘out’ of the nanopore. The mechanism can subsequently repeat with one or more further helicases, such that the conjugate is repetitively moved through the nanopore.

### Construct Assembly

The sequences of oligonucleotides used are outlined in Table 5. Multiple constructs were generated incorporating different peptide sequences. Peptides were synthesised with terminal modifications: N-terminal azide, and C-terminal methyltetrazine connected via an ethylenediamine linker. The constructs comprised a 3′ poly(dA) tail, which aids the loading of the helicase from solution, and a hairpin including a tethering oligo; both are ligated to the end of the construct. A diagram of the assembled construct is shown in FIG. 16. Constructs used in this Example were assembled according to the general method described in Example 1. A DNA oligo containing a terminal BCN click group and negatively charged C3 spacers (a; SEQ ID NO: 29) was first annealed to a tethering sequence (b; SEQ ID NO: 30). The tether increases analyte capture rate on the flowcell. The annealed oligos were then clicked to hairpin DNA (c; SEQ ID NO: 31) bearing an internal azide click group. The hairpin sequence contains a four base sticky-end onto which DNA 1 (d; SEQ ID NO: 32) and DNA 2 (e; SEQ ID NO: 33) oligos are ligated. The DNA 2 oligo has a terminal 3′ TCO group, which reacts with the tetrazine at the C-terminus of the peptide (f). The N-terminus of the peptide bears an azide group, which was then clicked to DNA 3 (g; SEQ ID NO: 34) through the terminal 5′ BCN group.

TABLE 5
oligonucleotides used in Example 3; corresponding
labels a-g are shown in FIG. 16.

| | |
|---|---|
| a | /5SpC3//iSpC3//iSpC3//iSpC3//iSpC3//iSpC3// |
| | iSpC3//iSpC3//iSpC3//iSpC3//iSpC3//iSpC3// |
| | iSpC3//iSpC3//iSpC3//iSpC3//iSpC3//iSpC3// |
| | iSpC3//iSpC3/GTTATTCAAGACTTCTTTAATACACTT |
| | T/BCN/ |
| b | GTCCATTATTCTTTTTTGCTGCGGCTTAGCTGTGATAGCAG |
| | GA |
| c | /5Phos/CGCAATACGTAACTGAACGAAGTACT/iBCN/ |
| | TGTACTTCGTTCAGTTACGTATTGCGTCCT |
| d | /5Phos/GTGTATTAAAGAAGTCTTGAATAACTTTGAGGCGA |
| | GCGGTCAA |
| e | /5Phos/GCTATCACAGCTAAGCCGCAGC/TCO/ |
| f | peptide with N-terminal azide |
| | and C-terminal tetrazine |
| g | /BCN/GAATAATGGACAAAAAAAAAAAAAAAAAAAAAAAAA |
| | AAAAAAAAAAAAAAAAAAAAAAAAAAAAAAAAAAAAAAAAA |
| | AAAAAAAAAAAAAA |

### Data Acquisition

Electrical data were collected using MinION flow cells (Oxford Nanopore Technologies plc) comprising modified Rhodococcus pores as used in Example 1. Samples were prepared by incubating 0.2 μM of polynucleotide-polypeptide conjugate with 1 μM wild-type Hel308 helicase in either Running Buffer (25 mM HEPES-KOH (pH 8.0), 500 mM KCl) or Sequencing Buffer (25 mM HEPES-KOH (pH 8.0), 350 mM KCl, 50 mM ATP, 50 mM MgCl2, 2.2 mM EDTA). The Running Buffer lacks ATP and was performed as a control for ATP-dependent enzymatic movement. Flow cells were first flushed with at least 1 mL of Running Buffer or Sequencing Buffer. Samples (75 μL) were then introduced into the flow cell via the SpotON port. Electrical data were acquired with a sample rate of 3 kHz and applied potential of 160 mV.

### Results and Discussion

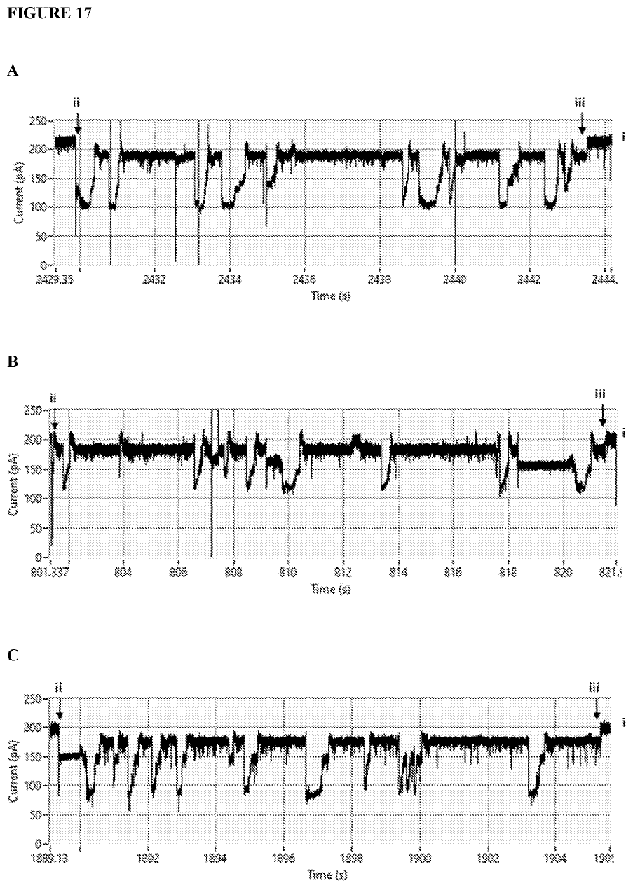

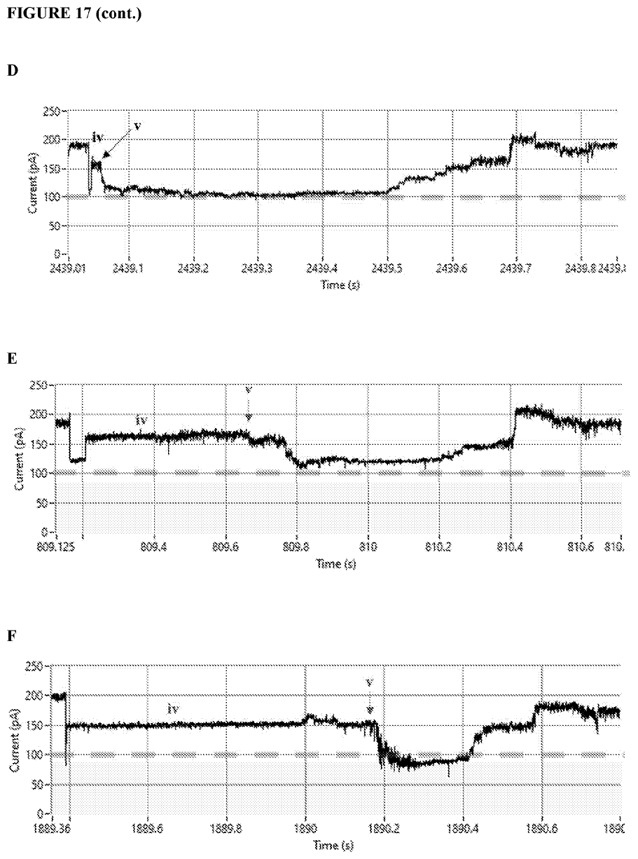

FIG. 17, A-F shows example current-time traces for enzymatically-controlled movement of the polypeptide-polynucleotide conjugate using Hel308 helicase. Three different peptide sequences were tested: RSDSGQQARY (SEQ ID NO: 35); GGSGSSSGSG (SEQ ID NO: 36); and EAIYAAPFAKKK (SEQ ID NO: 37) (see Figure Legend). Both re-reading and single-read traces are shown for each peptide. The experiments were performed under conditions of excess helicase with respect to conjugate. The presence of repetitive traces demonstrates that the conjugates were captured via the hairpin end and read multiple times. These traces demonstrate that peptides of different sequences produce unique signals.

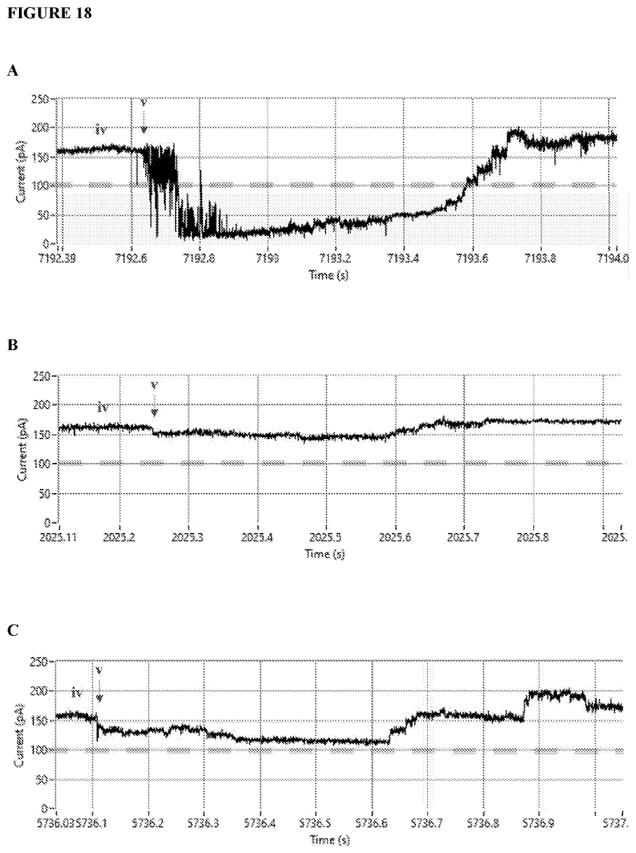

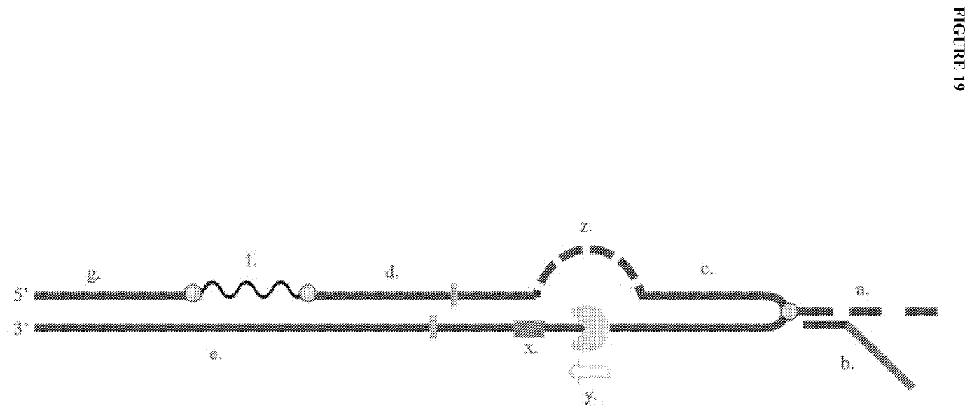

FIG. 18, A-C shows example current-time traces of peptides of varying charge, ranging from eight negatively charged residues to eight positively charged residues. The following peptides were tested:

A.
(SEQ ID NO: 38)
SRRRRRRRRS (charge: +8)
B.
(SEQ ID NO: 39)
SEEEEEEEES (charge: −8)
C.
(SEQ ID NO: 40)
HDSGYEVHHQK* (charge: −2).
*: indicates that the peptide was attached via
the C-terminal lysine R-group rather than via
the C-terminal carboxyl group.

The current-time traces shown in FIG. 18, A-C, demonstrate that it is possible to move peptides of varying charge through the nanopore. Most notably, peptides with a large positive charge (exemplified by peptide A with a charge of +8) can be moved through the pore against an electrophoretic force.

### Example 4

This example demonstrates ‘single-pass’ movement of a polynucleotide-polypeptide conjugate through a nanopore. The conjugate is moved in the direction ‘into’ the pore by a Dda helicase. The peptide is conjugated between two dsDNA oligonucleotides. The conjugate comprises a Dda helicase-loaded hairpin adapter, in which the helicase is topologically closed around a single-strand of polynucleotide ahead of a stall. The presence of the bound helicase initially prevents the conjugate fully translocating through the nanopore. Once the analyte enters the pore, the helicase is de-stalled by the electrophoretic force and moves in the direction 5′ to 3′ along a single strand of DNA to control the movement of dsDNA and peptide in the direction ‘into’ the nanopore. The helicase does not directly encounter the peptide, which is located on the complementary DNA “carrier” strand and moves through the pore along with the strand on which the helicase translocates. The helicase falls off the construct when it reaches the end of the single strand of polynucleotide on which it moves, releasing the remainder of the conjugate through the pore and terminating the read.

### Construct Assembly

The sequences of oligonucleotides used are outlined in Table 6. The constructs comprised an enzyme-loaded hairpin adapter with a tethering site. A diagram of the assembled construct is shown in FIG. 19. Constructs used in this Example were assembled according to the general method described in Example 1. A DNA oligo containing a terminal BCN click group and negatively charged C3 spacers (a; SEQ ID NO: 29) was first annealed to a tethering sequence (b; SEQ ID NO: 30). The tether increases analyte capture rate on the flowcell. The annealed oligos were then clicked to hairpin DNA (c; SEQ ID NO: 41) bearing an internal azide click group. The hairpin contains a Dda helicase loaded onto a single stranded portion and held in place by a disulphide bridge, which topologically closes the enzyme around the strand. The loading site is opposed by a nucleotide 2′-O-methyl RNA bubble to provide space for the enzyme to load. The use of RNA bases prevents the helicase from binding to the bubble instead of the intended loading site. The hairpin adapter contains a four base sticky-end onto which DNA 1 (d; SEQ ID NO: 42) and DNA 2 (e; SEQ ID NO: 43) oligos were ligated. The DNA 2 oligo has a terminal 3′ TCO group, which reacts with the tetrazine at the C-terminus of the peptide (f, SEQ ID NO: 44). The N-terminus of the peptide bears an azide group, which was then clicked to DNA 3 (g; SEQ ID NO: 45) through the terminal 5′ BCN group.

TABLE 6
oligonucleotides used in Example 4;
corresponding labels a-g are shown
in FIG. 19.

| | |
|---|---|
| a | /5SpC3//iSpC3//iSpC3//iSpC3//iSpC3//iSpC3 |
| | //iSpC3//iSpC3//iSpC3//iSpC3//iSpC3//iSpC3 |
| | //iSpC3//iSpC3//iSpC3//iSpC3//iSpC3//iSpC3 |
| | //iSpC3//iSpC3/GTTATTCAAGACTTCTTTAATACACT |
| | TT/BCN/ |
| b | GTCCATTATTCTTTTTTGCTGCGGCTTAGCTGTGATAGCAG |
| | GA |
| c | /5Phos/GCAATACGTAACTGAACGAAGT/iBNA-T//iBNA- |
| | G//iBNA-T//iBNA-G//iBNA-G/mUmUmUmUmUmUmUmU |
| | mUmUmUmUmUmUmUmUmUmUmUmUmUmUmUmUmUmUmUmUmU |
| | mUmUmUmUmUmUGGTTAAACACCCAAGCAGACGCCT7TGGCG |
| | TCTGCTTGGGTGTTTAACCTTTTTTTTTT8CCACAACTTCGT |
| | TCAGTTACGTATTGCTCCT |
| d | CTCAAGTGCCTGGTATATTACATCCACAGTGAAGACCTGGAC |
| | ACTGGACGTCCATTATTC/TCO/ |
| e | /5Phos/GCTATCACAGGCAATAAGAATAACGTCATAATGCG |
| | TAACTGACTAAGCCGCAGCTTTTTTGAATAATGGACGTCCAG |
| | TGTCCAGGTCTTCACTGTGGATGTAATATACCAGGCACTTGA |
| | G |
| f | peptide [HDSGDEVHHQK*] with N-terminal |
| | azide and C-terminal tetrazine |
| g | /BCN/GCTGCGGCTTAGTCAGTTACGCATTATGACGTTATTC |
| | TTATTGCCTGTGATAGCAGGA |

*indicates that the peptide was attached via the C-terminal lysine R-group rather than via the C-terminal carboxyl group.

### Data Acquisition

Electrical data were collected using MinION flow cells (Oxford Nanopore Technologies plc) comprising modified Rhodococcus pores as used in Example 1. Samples were prepared by mixing 0.2 μM of polynucleotide-polypeptide conjugate loaded with Dda helicase with Sequencing Buffer (25 mM HEPES-KOH (pH 8.0), 350 mM KCl, 50 mM ATP, 50 mM MgCl₂, 2.2 mM EDTA). Flow cells were first flushed with at least 1 mL of Sequencing Buffer. Samples (75 μL) were then introduced into the flow cell via the SpotON port. Electrical data were acquired with a sample rate of 1 kHz and applied potential of 180 mV. The recording temperature was 21 °C.

### Results and Discussion

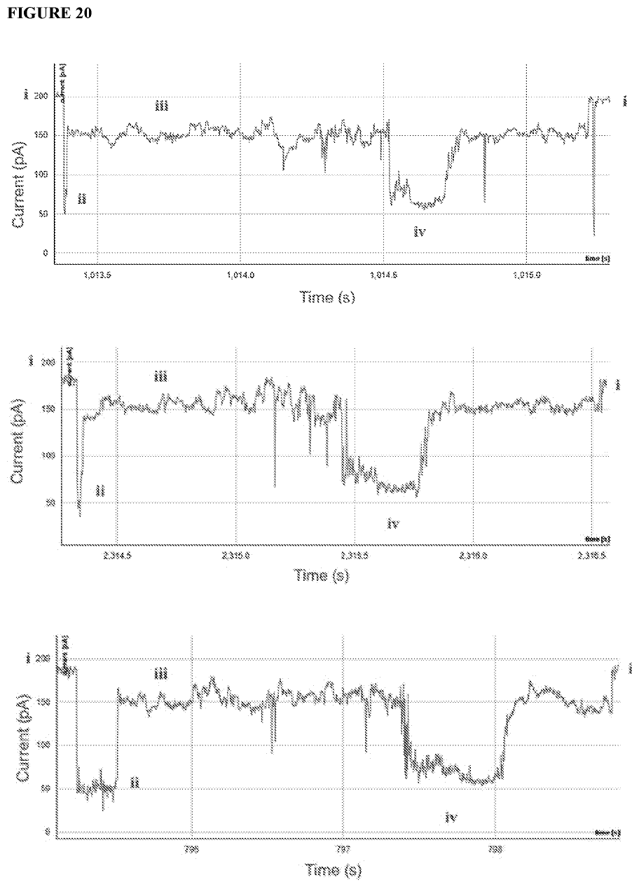

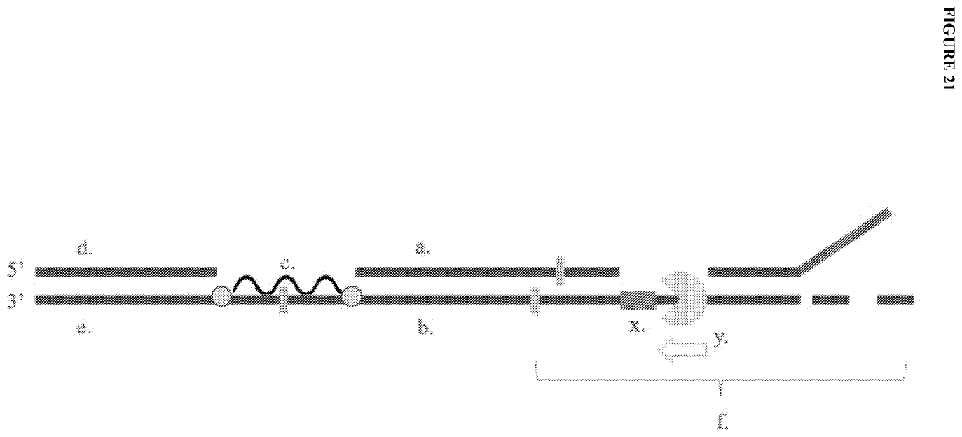

FIG. 20 shows example traces of enzymatically-controlled movement of the polypeptide-polynucleotide conjugate using Dda helicase in the single-pass mode. The presence of reproducible signal deviations from the dsDNA level (three examples shown) demonstrate that the peptide (HDSGDEVHHQK; SEQ ID NO: 46) on the “carrier” strand is co-translocated through the pore. Placing the peptide on the “carrier” can place fewer constraints on the length of the peptide which can be analysed, as the enzyme does not have to diffuse over the peptide to move the conjugate through the pore.

### Example 5

This example demonstrates ‘single-pass’ movement of a polynucleotide-polypeptide conjugate through a nanopore. Movement of the conjugate in the direction ‘into’ the nanopore is controlled by a Dda helicase enzyme. The peptide is conjugated between two double stranded (dsDNA) oligonucleotides, which contain internal rather than terminal click groups, creating a single-stranded stretch of DNA alongside the peptide (see FIG. 6). A sequencing adapter loaded with a helicase is attached to the end of the construct. The helicase is topologically closed around a single-stranded DNA portion of oligonucleotide ahead of a stall. The presence of the bound helicase initially prevents the conjugate fully translocating through the nanopore. Once the conjugate enters the nanopore, it is de-stalled by the electrophoretic force and the helicase then moves in the direction 5′ to 3′ along a single strand of DNA. The annealed complementary strand is ‘unzipped’ (separated) as the conjugate moves through the nanopore, such that only the strand of DNA with the attached peptide moves through the constriction. When the helicase encounters the covalently linked DNA-peptide segment, it diffuses over both strands, re-engaging on the DNA on the other side of the DNA-peptide segment. It then continues controlling the movement of the DNA-peptide followed by single-stranded DNA in a 5′ to 3′ direction through the nanopore. The enzyme falls off when it reaches the end of the bound DNA oligonucleotide, releasing the remainder of the conjugate through the nanopore and terminating the read. With this system, the nanopore only needs to have a diameter wide enough to accommodate co-translocating DNA-peptide (which may have across-section narrower than that of double-stranded DNA).

### Construct Assembly

The sequences of oligonucleotides used are outlined in Table 7. The constructs comprised an enzyme-loaded adapter with a tethering site ligated to the end of the construct, and the DNA spanning the peptide was composed of sections from two oligos, which may optionally be ligated together. A diagram of the assembled construct is shown in FIG. 21. Constructs used in this Example were assembled according to the general method described in Example 1. Two DNA oligos were annealed (a, b; SEQ ID NOS: 47, 48), one of which contained a 3′ TCO click group (b). The annealed oligos were then reacted with a peptide bearing a C-terminal tetrazine click group (c; SEQ ID NO: 49). The N-terminus of the peptide contained an azide group, which was then clicked to another set of annealed oligos (d, e; SEQ ID NOS: 50-51), one of which contained a 5′ BCN click group (e). The polynucleotide-polypeptide conjugate was ligated to a standard sequencing adapter (f) loaded with a topologically-closed helicase (using a disulphide closure) and containing a negatively charged leader sequence and a tether sequence. The tether increases analyte capture rate on the flowcell.

TABLE 7
oligonucleotides used in Example 5; corresponding labels a-e are shown in
FIG. 21. Also shown is the oligonucleotide for an alternative construct,
in which DNA oligo (b) and DNA oligo (e) are replaced by a single
oligonucleotide which spans the peptide
(Alternative strand: b + e; SEQ ID NO: 52).

| | |
|---|---|
| a | AAAAAAAAAAAAAAAAAAAAAAAAAGGTTAAACACCCAAGCA |
| | GCAAT |
| b | GCTTGGGTGTTTAACCTTTTTTTTTTTTTTTTTTTTTTTTT/iC3- |
| | TCO/TTT |
| c | Azide-HDSGDEVHHQK*- Tetrazine |
| d | CCCTGGACACTGGACAAAAAAAAAAAAAAAAAAAAAAAAA |
| e | /5Phos/TTT/iC3- |
| | BCN/TTTTTTTTTTTTTTTTTTTTTTTTTGTCCAGTGTCCAGGG |
| Alternative | CCCTGGACACTGGACAAAAAAAAAAAAAAAAAAAAAAAAAAA |
| strand: b + e | AAAAAAAAAAAAAAAAAAAAAAAAAAAAAAAGGTTAAACACC |
| | CAAGCAGCAAT |

*indicates that the peptide was attached via the C-terminal lysine R-group rather than via the C-terminal carboxyl group.

### Data Acquisition

Electrical data were collected using MinION flow cells (Oxford Nanopore Technologies plc) comprising modified Rhodococcus pores. Samples were prepared by mixing 0.2 μM of polynucleotide-polypeptide conjugate loaded with Dda helicase with Sequencing Buffer (25 mM HEPES-KOH (pH 8.0), 350 mM KCl, 50 mM ATP, 50 mM MgCl₂, 2.2 mM EDTA). Flow cells were first flushed with at least 1 mL of Sequencing Buffer. Samples (75 μL) were then introduced into the flow cell via the SpotON port. Electrical data were acquired with a sample rate of 1 kHz and applied potential of 200 mV. The recording temperature was 21 °C.

### Results and Discussion

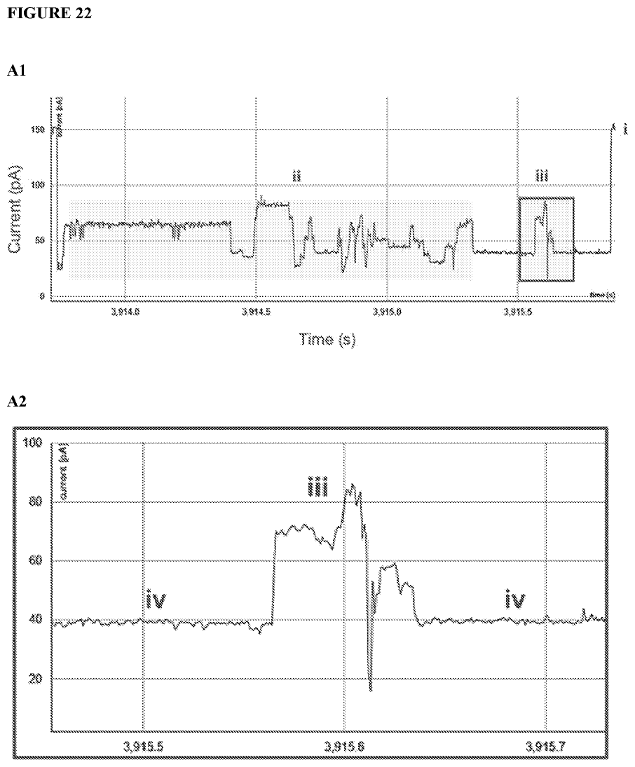

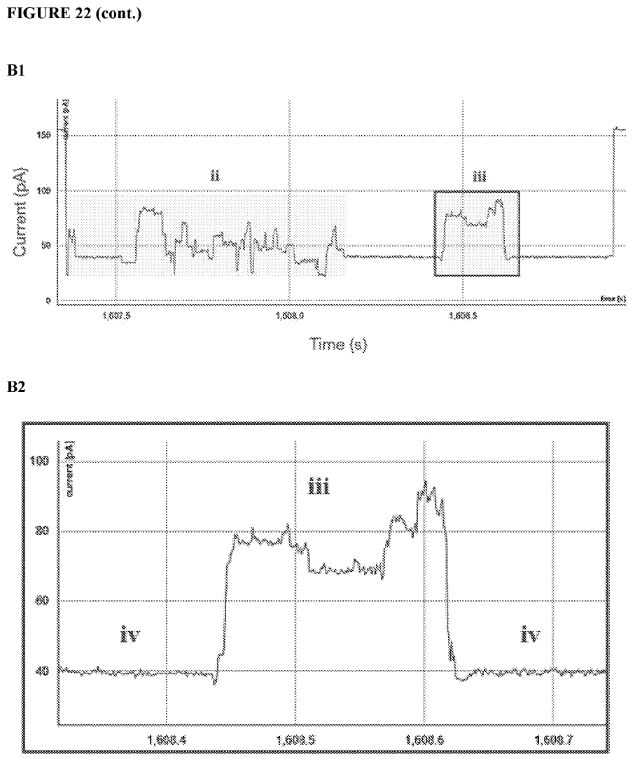

FIG. 22A shows an example current-time trace of Dda helicase-controlled movement of the polypeptide-polynucleotide conjugate, including co-translocation of DNA-peptide. The peptide (HDSGDEVHHQK) is covalently linked to the DNA at its C- and N-terminal ends, and therefore both DNA and peptide co-translocate through the nanopore together to produce the observed signal; see FIG. 22A1, 22A2.

A control experiment was carried out in which a peptide of the same sequence (HDSGDEVHHQK) was translocated alone, i.e. without being co-translocated with a strand of DNA, using a construct in which the peptide bridges a gap between two flanking DNA oligonucleotides (as described in WO 2021/111125). As shown in FIG. 22B1(iii) and 22B2(iii), the obtained current-time trace for the peptide section in this control experiment is different from that shown in FIG. 22A1(iii) and 22A2(iii), thus confirming that co-translocation of DNA and peptide is shown in FIG. 22A1 and 22A2, and demonstrating that the presence of negative charge in the co-translocating DNA modulates the peptide signal.

### Example 6

This example demonstrates ‘single-pass’ movement of a polynucleotide-polypeptide conjugate through a nanopore. Movement of the conjugate in the direction ‘into’ the nanopore is controlled by a Dda helicase enzyme. The peptide is conjugated between two double stranded (dsDNA) oligonucleotides, one strand of which contains an internal rather than terminal click group, creating a ‘flap’ of single-stranded DNA alongside the peptide. A standard sequencing adapter loaded with a helicase is attached to the end of the construct. The helicase is topologically closed around a single-stranded DNA portion of oligonucleotide ahead of a stall. The presence of the bound helicase initially prevents the conjugate fully translocating through the nanopore. Once the conjugate enters the nanopore, it is de-stalled by the electrophoretic force and the helicase then moves 5′-3′ along a single strand of DNA. The annealed complementary strand is ‘unzipped’ (separated) as the conjugate moves through the nanopore, such that only the strand of DNA with the attached peptide moves through the constriction. When the helicase encounters the covalently linked DNA-peptide segment, it diffuses over both the peptide and the DNA ‘flap’, re-engaging on the DNA on the other side of the DNA-peptide segment. It then continues controlling the movement of the DNA-peptide followed by single-stranded DNA in a 5′ to 3′ direction through the nanopore. The enzyme falls off when it reaches the end of the bound DNA oligonucleotide, releasing the remainder of the conjugate through the nanopore and terminating the read. As in Example 5 above, with this system, the nanopore only needs to have a diameter wide enough to accommodate co-translocating DNA-peptide.

### Construct Assembly

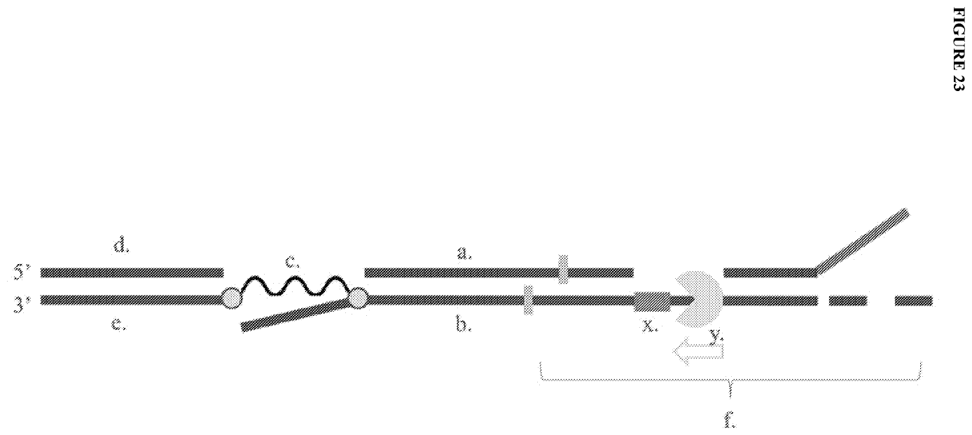

The sequences of oligonucleotides used are outlined in Table 8. The constructs comprised an enzyme-loaded adapter with a tethering site ligated to the end of the construct, and a DNA ‘flap’ spanning the peptide composed of 6 bases, attached to the C-terminus of the peptide. A diagram of the assembled construct is shown in FIG. 23. Constructs used in this Example were assembled according to the general method described in Example 1. Two DNA oligos were annealed (a, b; SEQ ID NOS: 53, 54), one of which contained an internal 3′ TCO click group (b). The annealed oligos were then reacted with a peptide bearing a C-terminal tetrazine click group (c; SEQ ID NO: 49). The N-terminus of the peptide contains an azide group, which was then clicked to another set of annealed oligos (d, e; SEQ ID NOS: 55, 56), one of which contained a terminal 5′ BCN click group (e). The polynucleotide-polypeptide conjugate was then ligated to a standard sequencing adapter (f), loaded with a topologically-closed helicase (using a disulphide closure) and containing a negatively charged leader sequence and a tether sequence. The tether increases analyte capture rate on the flowcell.

TABLE 8
oligonucleotides used in Example 6;
corresponding labels a-f are shown in FIG. 23.

| | |
|---|---|
| a | AAAAAAAAAAAAAAAAAAAAAAAAAAAAAAAAAAAA |
| | AAAAGGTTAAACACCCAAGCAGCAAT |
| b | GCTTGGGTGTTTAACCTTTTTTTTTTTTTTTTTTTT |
| | TTTTTTTTTTTTTTTTTTTT/iC3-TCO/TTTTTT |
| c | Azide-HDSGDEVHHQK *- Tetrazine |
| d | CCCAAAAAAAAAAAAAAAAAAAAAAAAAAAAAAAAA |
| | AAAAAAAAAAAAAAAAAAAAAAA |
| e | /BCN/TTTTTTTTTTTTTTTTTTTTTTTTTTTTTTT |
| | TTTTTTTTTTTTTTTTTTTTTTTTTGGG |
| f | Sequencing adapter loaded with helicase |

*indicates that the peptide was attached via the C-terminal lysine R-group rather than via the C-terminal carboxyl group.

### Data Acquisition

Electrical data were collected using MinION flow cells (Oxford Nanopore Technologies plc) comprising modified Rhodococcus pores. Samples were prepared by mixing 0.2 μM of polynucleotide-polypeptide conjugate loaded with Dda helicase with Sequencing Buffer (25 mM HEPES-KOH (pH 8.0), 350 mM KCl, 50 mM ATP, 50 mM MgCl₂, 2.2 mM EDTA). Flow cells were first flushed with at least 1 mL of Sequencing Buffer. Samples (75 μL) were then introduced into the flow cell via the SpotON port. Electrical data were acquired with a sample rate of 1 kHz and applied potential of 200 mV. The recording temperature was 21 °C.

### Results and Discussion

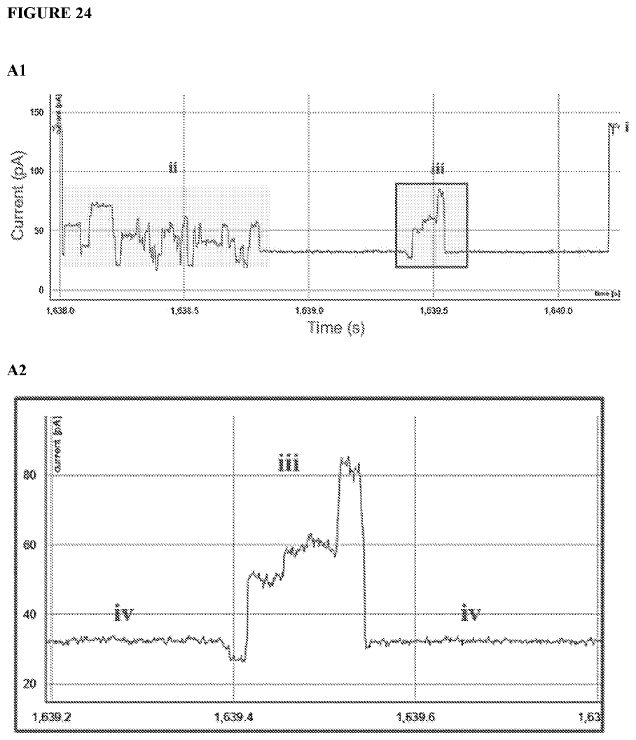

FIG. 24 shows a current-time trace of Dda helicase-controlled movement of the polypeptide-polynucleotide conjugate, including co-translocation of the DNA flap and peptide. The peptide (HDSGDEVHHQK) is covalently linked to the DNA flap at its C-terminus and passes through the nanopore from C- to N-terminus; thus both DNA flap and peptide co-translocate through the nanopore together to produce the observed signal. The peptide signal produced by this scheme differs from that produced by the control experiment of peptide-only translocation (using a corresponding construct comprising the same peptide sequence HDSGDEVHHQK but lacking the DNA flap), as described above in Example 5 and shown in FIG. 22B.

### Description of the Sequence Listing

SEQ ID NO: 1 shows the amino acid sequence of (hexa-histidine tagged) exonuclease I(EcoExo I) from E. coli.

SEQ ID NO: 2 shows the amino acid sequence of the exonuclease III enzyme from E. coli.

SEQ ID NO: 3 shows the amino acid sequence of the RecJ enzyme from T. thermophilus (TthRecJ-cd).

SEQ ID NO: 4 shows the amino acid sequence of bacteriophage lambda exonuclease. The sequence is one of three identical subunits that assemble into a trimer. (http://www.neb.com/nebecomm/products/productM0262.asp).

SEQ ID NO: 5 shows the amino acid sequence of Phi29 DNA polymerase from Bacillus subtilis.

SEQ ID NO: 6 shows the amino acid sequence of Trwc Cba (Citromicrobium bathyomarinum) helicase.

SEQ ID NO: 7 shows the amino acid sequence of Hel308 Mbu (Methanococcoides burtonii) helicase.

SEQ ID NO: 8 shows the amino acid sequence of the Dda helicase 1993 from Enterobacteria phage T4.

SEQ ID NO: 9 shows the amino acid sequence of a monomer of the transmembrane protein nanopore derived from Rhodococcus, as described in Example 1.

SEQ ID NOs: 10-12 show polypeptide sequences (read N-to-C) used in Example 1.

SEQ ID NOs: 13-26 show polynucleotide sequences used in Example 1. 3 denotes an amino C3 labelled with TCO. 2 denotes a 5′ amino C3 labelled with BCN, 1 denotes a 3′ Biotin modification. “+” before a base denotes LNA.

SEQ ID NOs: 27-28 show polynucleotide sequences used in Example 2. 3 denotes an amino C3 labelled with TCO. 2 denotes a 5′ amino C3 labelled with BCN. “+” before abase denotes LNA.

SEQ ID NOs: 29-56 show polynucleotide and polypeptide sequences used in Examples 3 to 6.

1. A method of characterising a target polypeptide, comprising
contacting (i) a polynucleotide-polypeptide conjugate strand comprising a target polypeptide conjugated at each end of said target polypeptide to one or more polynucleotide flanking strands with (ii) a polynucleotide carrier strand, thereby forming a polynucleotide-polypeptide construct;
contacting the construct with a nanopore under conditions such that both the polynucleotide-polypeptide conjugate strand and the polynucleotide carrier strand co-translocate through the nanopore; and
taking one or more measurements characteristic of the polypeptide as the construct moves with respect to the nanopore,
thereby characterising the target polypeptide.

2. A method according to claim 1, wherein the one or more polynucleotide flanking strands are each independently complementary to a region of the polynucleotide carrier strand.

3. A method according to claim 1 or claim 2, wherein the one or more polynucleotide flanking strands are each independently at least partially hybridized to the polynucleotide carrier strand.

4. A method of characterising a target polypeptide, comprising
contacting (i) a polynucleotide-polypeptide conjugate strand comprising a target polypeptide attached to a polynucleotide flanking strand with (ii) a polynucleotide-handling protein capable of controlling the movement of the polynucleotide flanking strand with respect to a nanopore; and
contacting the polynucleotide-polypeptide conjugate strand with a nanopore under conditions such that the polynucleotide-handling protein controls the movement of the polynucleotide-polypeptide conjugate strand with respect to the nanopore; and
taking one or more measurements characteristic of the polypeptide as the polynucleotide flanking strand and the target polypeptide co-translocate through the nanopore,
thereby characterising the target polypeptide.

5. A method according to claim 4, wherein prior to said method the polynucleotide flanking strand is at least partially hybridized to a polynucleotide carrier strand thereby forming a polynucleotide-polypeptide construct.

6. A method according to any one of the preceding claims, wherein the polynucleotide-polypeptide conjugate strand comprises a plurality of target polypeptides.

7. A method according to any one of the preceding claims, wherein during said method the or each polypeptide is independently held in a linearized form.

8. A method according to any one of the preceding claims, wherein the or each target polypeptide independently has a length of from about 5 to about 1000 peptide units.

9. A method according to any one of the preceding claims, comprising mechanically manipulating said construct, polynucleotide-polypeptide conjugate strand and/or polynucleotide carrier strand thereby moving said construct, polynucleotide-polypeptide conjugate strand and/or polynucleotide carrier strand with respect to the nanopore.

10. A method according to claim 9, wherein the construct, polynucleotide-polypeptide conjugate strand and/or polynucleotide carrier strand is moved by mechanical manipulation in a direction opposite to a potential applied across said nanopore.

11. A method according to claim 10, wherein said potential is a voltage potential applied across said nanopore.

12. A method according to any one of claims 1 to 3 or 5 to 11, comprising contacting the construct with a polynucleotide-handling protein capable of controlling the movement of the one or more polynucleotide flanking strands and/or the polynucleotide carrier strand, and wherein the polynucleotide-handling protein controls the movement of the construct with respect to the nanopore.

13. A method according to claim 12, comprising contacting both polynucleotide-polypeptide conjugate strand and the polynucleotide carrier strand of the construct with a polynucleotide-handling protein capable of controlling the movement of the polynucleotide-polypeptide conjugate strand and/or the polynucleotide carrier strand, and wherein the polynucleotide-handling protein controls the movement of the construct with respect to the nanopore.

14. A method according to any one of claims 1 to 11, comprising contacting the polynucleotide-polypeptide conjugate strand with a polynucleotide-handling protein capable of controlling the movement of the polynucleotide-polypeptide conjugate strand, and wherein the polynucleotide-handling protein controls the movement of the construct with respect to the nanopore.

15. A method according to any one of claims 1 to 3 or 5 to 11, comprising contacting the polynucleotide carrier strand with a polynucleotide-handling protein capable of controlling the movement of the polynucleotide carrier strand, and wherein the polynucleotide-handling protein controls the movement of the construct with respect to the nanopore.

16. A method according to any one of claims 12 to 15, wherein:
i) the polynucleotide-handling protein is located on the cis side of the nanopore and the polynucleotide-handling protein controls the movement of the construct, polynucleotide-polypeptide conjugate strand and/or polynucleotide carrier strand from the cis side of the nanopore to the trans side of the nanopore thereby controlling the movement of the target polypeptide from the cis side of the nanopore to the trans side of the nanopore; or
ii) the polynucleotide-handling protein is located on the trans side of the nanopore and the polynucleotide-handling protein controls the movement of the construct, polynucleotide-polypeptide conjugate strand and/or polynucleotide carrier strand from the trans side of the nanopore to the cis side of the nanopore thereby controlling the movement of the target polypeptide from the trans side of the nanopore to the cis side of the nanopore.

17. A method according to claim 6, wherein the polynucleotide-handling protein is located on the cis side of the nanopore and the polynucleotide-handling protein controls the movement of the polynucleotide carrier strand from the cis side of the nanopore to the trans side of the nanopore, thereby controlling the movement of the target polypeptide through the nanopore.

18. A method according to claim 16, wherein the polynucleotide-handling protein is located on the trans side of the nanopore and the polynucleotide-handling protein controls the movement of the polynucleotide carrier strand from the trans side of the nanopore to the cis side of the nanopore, thereby controlling the movement of the target polypeptide through the nanopore.

19. A method according to any one of claims 12 to 15, wherein:
i) the polynucleotide-handling protein is located on the cis side of the nanopore and the polynucleotide-handling protein controls the movement of the construct, polynucleotide-polypeptide conjugate strand and/or polynucleotide carrier strand from the trans side of the nanopore to the cis side of the nanopore thereby controlling the movement of the target polypeptide from the trans side of the nanopore to the cis side of the nanopore; or
ii) the polynucleotide-handling protein is located on the trans side of the nanopore and the polynucleotide-handling protein controls the movement of the construct, polynucleotide-polypeptide conjugate strand and/or polynucleotide carrier strand from the cis side of the nanopore to the trans side of the nanopore thereby controlling the movement of the target polypeptide from the cis side of the nanopore to the trans side of the nanopore.

20. A method according to claim 19, wherein the polynucleotide-handling protein is located on the cis side of the nanopore and the polynucleotide-handling protein controls the movement of the polynucleotide carrier strand from the trans side of the nanopore to the cis side of the nanopore, thereby controlling the movement of the target polypeptide through the nanopore.

21. A method according to claim 19, wherein the polynucleotide-handling protein is located on the trans side of the nanopore and the polynucleotide-handling protein controls the movement of the polynucleotide carrier strand from the cis side of the nanopore to the trans side of the nanopore, thereby controlling the movement of the target polypeptide through the nanopore.

22. A method according to any one of claims 12 to 21, wherein prior to the contacting of the construct with the nanopore the polynucleotide-handling protein is bound to the polynucleotide carrier strand in a region of the polynucleotide carrier strand that is spanned by a non-hybridised region of the polynucleotide flanking strand.

23. A method according to any one of claims 12 to 21, wherein the polynucleotide-handling protein is capable of remaining bound to the polynucleotide-polypeptide conjugate strand when the portion of the polynucleotide-polypeptide conjugate strand in contact with the active site of the polynucleotide-handling protein comprises the target polypeptide.

24. A method according to any one of claims 12 to 23, wherein the polynucleotide-handling protein is modified to prevent it from disengaging from the construct, polynucleotide-polypeptide conjugate strand and/or polynucleotide carrier strand when the polynucleotide-handling protein contacts the target polypeptide.

25. A method according to any one of claims 12 to 24, wherein the polynucleotide-handling protein is modified to wholly or partially close an opening existing in at least one conformation state of the unmodified protein through which a polynucleotide strand can unbind.

26. A method according to any one of claims 12 to 25, wherein the polynucleotide-handling protein is or comprises a helicase, translocase or helicase-nuclease complex.

27. A method according to any one of the preceding claims, wherein the construct comprises a stalling moiety and prior to the translocation of the target polypeptide through the nanopore the polynucleotide-handling protein is positioned such that the stalling moiety is located between the polynucleotide-handling protein and the target polypeptide.

28. A method according to any one of the preceding claims, wherein one or more adapters and/or one or more tethers and/or one or more anchors are attached to the construct, polynucleotide-polypeptide conjugate strand and/or polynucleotide carrier strand.

29. A method according to any one of the preceding claims, wherein the construct, polynucleotide-polypeptide conjugate strand and/or polynucleotide carrier strand comprises a blocking moiety attached via an optional linker, wherein the blocking moiety is incapable of translocating through the nanopore.

30. A method according to any one of the preceding claims, wherein the method comprises:
i) carrying out the method according to any one of the preceding claims such that the target polypeptide translocates the nanopore in a first direction with respect to the nanopore;
ii) allowing the construct, polynucleotide-polypeptide conjugate strand and/or polynucleotide carrier strand to move in a direction opposite to the direction of movement with respect to the nanopore in step (i) such that the target polypeptide translocates the nanopore in a second direction which is opposite to the first direction;
iii) optionally allowing the construct, polynucleotide-polypeptide conjugate strand and/or polynucleotide carrier strand to move in the first direction such that the target polypeptide re-translocates the nanopore in the first direction;
iv) optionally repeating steps (ii) and (iii) to oscillate the polypeptide through the nanopore.

31. A method according to any one of the preceding claims, wherein the one or more measurements are characteristic of one or more characteristics of the target polypeptide selected from (i) the length of the target polypeptide, (ii) the identity of the target polypeptide, (iii) the sequence of the target polypeptide, (iv) the secondary structure of the target polypeptide and (v) whether or not the target polypeptide is modified.

32. A method according to any one of the preceding claims, wherein the nanopore is a protein nanopore, preferably a β-barrel protein nanopore.

33. A system comprising
a construct comprising (i) a polynucleotide-polypeptide conjugate strand comprising a target polypeptide conjugated at each end of said target polypeptide to one or more polynucleotide flanking strands and (ii) a polynucleotide carrier strand;
a nanopore capable of co-translocating the polynucleotide-polypeptide conjugate strand and the polynucleotide flanking strand of the construct; and
a polynucleotide-handling protein.

34. A kit comprising:
a nanopore;
a first polynucleotide comprising a reactive functional group for conjugating to a first end of a target polypeptide;
a second polynucleotide comprising a reactive functional group for conjugating to a second end of the target polypeptide; and
a polynucleotide-handling protein.

35. A system according to claim 33 or kit according to claim 34, wherein the nanopore, construct and/or polynucleotide-handling protein are as defined in any one of claims 2 to 32.


SEQUENCE LISTING (61 sequences)

SEQ ID NO: 1 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 2 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 3 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 4 - protein, 226 aa, Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 5 - protein, 608 aa, Bacillus subtilis phage phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 6 - protein, 970 aa, Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 7 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 8 - protein, 439 aa, Enterobacteria phage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9 - protein, 203 aa, synthetic construct
MAVDDSNSVVDGGGNTITVSQSDTFINSVFPLDGSPLTREWFHNGRAIVDVTGPDAEDFSGTVTIGYQVGYPASLGGRLTFSYTTPGLNLSVGNGVAATVTNVLPQAGVGVTLTPGPGIETVAVASGAASGAHTEIQIANLHGTATKIAGNVSVRPYVQVVSSNGDVATTFGQPWRFNGSGGENLYFQGSGSGSAWSHPQFEK

SEQ ID NO: 10 - protein, 10 aa, synthetic construct
GGSGDDSGSG

SEQ ID NO: 11 - protein, 10 aa, synthetic construct
GGSGRRSGSG

SEQ ID NO: 12 - protein, 10 aa, synthetic construct
GGSGYYSGSG

SEQ ID NO: 13 - DNA, 60 bp, synthetic construct
  source 1..60: mol_type = other DNA
  modified_base 1..6: mod_base = OTHER; note = Locked nucleic acids
  misc_feature 60: note = amino C3 labelled with TCO
gtccacttttttgctgcggcttagctgtgatagcttttgctatcacagctaagccgcagc

SEQ ID NO: 14 - DNA, 65 bp, synthetic construct
  source 1..65: mol_type = other DNA
  misc_feature 65: note = amino C3 labelled with TCO
gtccattattcttttttgctgcggcttagctgtgatagcttttgctatcacagctaagccgcagc

SEQ ID NO: 15 - DNA, 70 bp, synthetic construct
  source 1..70: mol_type = other DNA
  misc_feature 70: note = amino C3 labelled with TCO
gtattgtccattattcttttttgctgcggcttagctgtgatagcttttgctatcacagctaagccgcagc

SEQ ID NO: 16 - DNA, 75 bp, synthetic construct
  source 1..75: mol_type = other DNA
  misc_feature 75: note = amino C3 labelled with TCO
gtattgtattgtccattattcttttttgctgcggcttagctgtgatagcttttgctatcacagctaagccgcagc

SEQ ID NO: 17 - DNA, 80 bp, synthetic construct
  source 1..80: mol_type = other DNA
  misc_feature 80: note = amino C3 labelled with TCO
gtattgtattgtattgtccattattcttttttgctgcggcttagctgtgatagcttttgctatcacagctaagccgcagc

SEQ ID NO: 18 - DNA, 85 bp, synthetic construct
  source 1..85: mol_type = other DNA
  misc_feature 85: note = amino C3 labelled with TCO
gtattgtattgtattgtattgtccattattcttttttgctgcggcttagctgtgatagcttttgctatcacagctaagccgcagc

SEQ ID NO: 19 - DNA, 90 bp, synthetic construct
  source 1..90: mol_type = other DNA
  misc_feature 90: note = amino C3 labelled with TCO
gtattgtattgtattgtattgtattgtccattattcttttttgctgcggcttagctgtgatagcttttgctatcacagctaagccgcagc

SEQ ID NO: 20 - skipped

SEQ ID NO: 21 - DNA, 11 bp, synthetic construct
  source 1..11: mol_type = other DNA
  misc_feature 1: note = 5' amino C3 labelled with BCN
  misc_feature 11: note = 3' Biotin
gaataatggac

SEQ ID NO: 22 - DNA, 16 bp, synthetic construct
  source 1..16: mol_type = other DNA
  misc_feature 16: note = 3' Biotin
  misc_feature 1: note = 5' amino C3 labelled with BCN
gaataatggacaatac

SEQ ID NO: 23 - DNA, 21 bp, synthetic construct
  source 1..21: mol_type = other DNA
  misc_feature 1: note = 5' amino C3 labelled with BCN
  misc_feature 21: note = 3' Biotin
gaataatggacaatacaatac

SEQ ID NO: 24 - DNA, 26 bp, synthetic construct
  source 1..26: mol_type = other DNA
  misc_feature 26: note = 3' Biotin
  misc_feature 1: note = 5' amino C3 labelled with BCN
gaataatggacaatacaatacaatac

SEQ ID NO: 25 - DNA, 31 bp, synthetic construct
  source 1..31: mol_type = other DNA
  misc_feature 1: note = 5' amino C3 labelled with BCN
  misc_feature 31: note = 3' Biotin
gaataatggacaatacaatacaatacaatac

SEQ ID NO: 26 - DNA, 36 bp, synthetic construct
  source 1..36: mol_type = other DNA
  misc_feature 1: note = 5' amino C3 labelled with BCN
  misc_feature 36: note = 3' Biotin
gaataatggacaatacaatacaatacaatacaatac

SEQ ID NO: 27 - DNA, 65 bp, synthetic construct; identical to SEQ ID NO: 14
  source 1..65: mol_type = other DNA
  modified_base 1..5: mod_base = OTHER; note = Locked nucleic acid
  misc_feature 65: note = amino C3 labelled with TCO

SEQ ID NO: 28 - DNA, 40 bp, synthetic construct
  source 1..40: mol_type = other DNA
  misc_feature 1: note = 5' amino C3 labelled with BCN
gaataatggacaaaaaaaaaaaaaaaaaaaaaaaaaaaaa

SEQ ID NO: 29 - DNA, 28 bp, synthetic construct
  source 1..28: mol_type = other DNA
  misc_feature 1: note = 20 5' iSpC3 groups
  misc_feature 28: note = 3' BCN
gttattcaagacttctttaatacacttt

SEQ ID NO: 30 - DNA, 43 bp, synthetic construct
  source 1..43: mol_type = other DNA
gtccattattcttttttgctgcggcttagctgtgatagcagga

SEQ ID NO: 31 - DNA, 56 bp, synthetic construct
  source 1..56: mol_type = other DNA
cgcaatacgtaactgaacgaagtacttgtacttcgttcagttacgtattgcgtcct

SEQ ID NO: 32 - DNA, 43 bp, synthetic construct
  source 1..43: mol_type = other DNA
gtgtattaaagaagtcttgaataactttgaggcgagcggtcaa

SEQ ID NO: 33 - DNA, 22 bp, synthetic construct
  source 1..22: mol_type = other DNA
  misc_feature 22: note = 3' TCO
gctatcacagctaagccgcagc

SEQ ID NO: 34 - DNA, 91 bp, synthetic construct
  source 1..91: mol_type = other DNA
  misc_feature 1: note = 5' BCN
gaataatggacaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - protein, 10 aa, synthetic construct
RSDSGQQARY

SEQ ID NO: 36 - protein, 10 aa, synthetic construct
GGSGSSSGSG

SEQ ID NO: 37 - protein, 12 aa, synthetic construct
EAIYAAPFAKKK

SEQ ID NO: 38 - protein, 10 aa, synthetic construct
SRRRRRRRRS

SEQ ID NO: 39 - protein, 10 aa, synthetic construct
SEEEEEEEES

SEQ ID NO: 40 - protein, 11 aa, synthetic construct
HDSGYEVHHQK

SEQ ID NO: 41 - DNA, 151 bp, synthetic construct
  source 1..151: mol_type = other DNA
  modified_base 23..27: mod_base = OTHER; note = Bridged nucleic acid
  modified_base 28..62: mod_base = um
gcaatacgtaactgaacgaagttgtggtttttttttttttttttttttttttttttttttttggttaaacacccaagcagacgccttggcgtctgcttgggtgtttaaccttttttttttccacaacttcgttcagttacgtattgctcct

SEQ ID NO: 42 - DNA, 60 bp, synthetic construct
  source 1..60: mol_type = other DNA
  misc_feature 60: note = 3' TCO
ctcaagtgcctggtatattacatccacagtgaagacctggacactggacgtccattattc

SEQ ID NO: 43 - DNA, 120 bp, synthetic construct
  source 1..120: mol_type = other DNA
gctatcacaggcaataagaataacgtcataatgcgtaactgactaagccgcagcttttttgaataatggacgtccagtgtccaggtcttcactgtggatgtaatataccaggcacttgag

SEQ ID NO: 44 - protein, 11 aa, synthetic construct
  MOD_RES 1: note = N-terminal azide
  MOD_RES 11: note = C-terminal tetrazine
HDSGDEVHHQK

SEQ ID NO: 45 - DNA, 58 bp, synthetic construct
  source 1..58: mol_type = other DNA
  misc_feature 1: note = 5' BCN
gctgcggcttagtcagttacgcattatgacgttattcttattgcctgtgatagcagga

SEQ ID NO: 46 - protein, 11 aa, synthetic construct
HDSGDEVHHQK

SEQ ID NO: 47 - DNA, 47 bp, synthetic construct
  source 1..47: mol_type = other DNA
aaaaaaaaaaaaaaaaaaaaaaaaaggttaaacacccaagcagcaat

SEQ ID NO: 48 - DNA, 44 bp, synthetic construct
  source 1..44: mol_type = other DNA
gcttgggtgtttaacctttttttttttttttttttttttttttt

SEQ ID NO: 49 - protein, 11 aa, synthetic construct
  MOD_RES 1: note = N-terminal azide
  MOD_RES 11: note = C-terminal tetrazine
HDSGDEVHHQK

SEQ ID NO: 50 - DNA, 40 bp, synthetic construct
  source 1..40: mol_type = other DNA
ccctggacactggacaaaaaaaaaaaaaaaaaaaaaaaaa

SEQ ID NO: 51 - DNA, 43 bp, synthetic construct
  source 1..43: mol_type = other DNA
ttttttttttttttttttttttttttttgtccagtgtccaggg

SEQ ID NO: 52 - DNA, 95 bp, synthetic construct
  source 1..95: mol_type = other DNA
ccctggacactggacaaaaaaaaaaaaaaaaaaaaaaaaaaaaaaaaaaaaaaaaaaaaaaaaaaaaaaaaaaggttaaacacccaagcagcaat

SEQ ID NO: 53 - DNA, 62 bp, synthetic construct
  source 1..62: mol_type = other DNA
aaaaaaaaaaaaaaaaaaaaaaaaaaaaaaaaaaaaaaaaggttaaacacccaagcagcaat

SEQ ID NO: 54 - DNA, 62 bp, synthetic construct
  source 1..62: mol_type = other DNA
gcttgggtgtttaacctttttttttttttttttttttttttttttttttttttttttttttt

SEQ ID NO: 55 - DNA, 59 bp, synthetic construct
  source 1..59: mol_type = other DNA
cccaaaaaaaaaaaaaaaaaaaaaaaaaaaaaaaaaaaaaaaaaaaaaaaaaaaaaaaa

SEQ ID NO: 56 - DNA, 59 bp, synthetic construct
  source 1..59: mol_type = other DNA
  misc_feature 1: note = 5' BCN
ttttttttttttttttttttttttttttttttttttttttttttttttttttttttggg

SEQ ID NO: 57 - DNA, 26 bp, synthetic construct
  source 1..26: mol_type = other DNA
cgcaatacgtaactgaacgaagtact

SEQ ID NO: 58 - DNA, 30 bp, synthetic construct
  source 1..30: mol_type = other DNA
tgtacttcgttcagttacgtattgcgtcct

SEQ ID NO: 59 - DNA, 41 bp, synthetic construct
  source 1..41: mol_type = other DNA
gcttgggtgtttaaccttttttttttttttttttttttttt

SEQ ID NO: 60 - DNA, 40 bp, synthetic construct
  source 1..40: mol_type = other DNA
tttttttttttttttttttttttttgtccagtgtccaggg

SEQ ID NO: 61 - DNA, 56 bp, synthetic construct
  source 1..56: mol_type = other DNA
gcttgggtgtttaacctttttttttttttttttttttttttttttttttttttttt